(19) United States
(12) Patent Application Publication
(10) Pub. No.: US 2008/0138352 A1
(43) Pub. Date: Jun. 12, 2008

(54) Identification and characterization of novel proline racemases and hydroxyproline-2-epimerases, uses thereof, and methods of identifying proline racemases and hydroxyproline-2-epimerases

(76) Inventors: Paola Minoprio, Villiers sur Marne (FR); Maira Goytia, Paris (FR); Nathalie Chamond, Paris (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/896,596

(22) Filed: Sep. 4, 2007

Related U.S. Application Data
(63) Continuation-in-part of application No. 10/545,149, filed on Aug. 15, 2006.
(60) Provisional application No. 60/446,263, filed on Feb. 11, 2003.

(51) Int. Cl. A61K 39/40; G06G 7/48; C12N 9/90; A61K 39/02; C07K 16/00; C07H 21/04; C12N 15/00; A61K 39/005; G01N 33/569; A61K 38/16

(52) U.S. Cl. 424/167.1; 703/11; 435/233; 435/4; 424/190.1; 424/191.1; 530/387.9; 435/7.32; 435/7.2; 435/253.3; 536/23.7; 435/6

(57) ABSTRACT

This invention provides identification and characterization of racemases and epimerases and definition of protein signatures of those racemases and epimerases. This invention also provides identification of nucleic acid molecules encoding a peptide consisting of a motif characteristic of the protein signatures, and to the peptides consisting of these motifs. Antibodies specific for the peptides and to immune complexes of these antibodies with the peptides are also provided. Further, the invention relates to methods and kits for detecting racemases and epimerases using the nucleic acid molecules of the invention, as well as the peptides consisting of the motifs and antibodies to these peptides.

This application is a continuation in part of U.S. patent application Ser. No. 10/545,149, filed Aug. 15, 2006, which is a National Stage Entry of PCT/IB04/00861, filed Feb. 11, 2004, which is based on and claims the benefit of U.S. Provisional Application Ser. No. 60/446,263, filed Feb. 11, 2003. The entire disclosures of each of these applications is relied upon and incorporated by reference herein.

### BACKGROUND OF THE INVENTION

This invention relates to the identification and characterization of racemases and epimerases and definition of protein signatures of those racemases and epimerases. This invention also relates to the identification of nucleic acid molecules encoding a peptide consisting of a motif characteristic of the protein signatures, and to the peptides consisting of these motifs. In addition, this invention relates to a process of production of D-amino acids using a eukaryotic amino acid racemase or a eukaryotic amino acid epimerase. This invention also relates to the use of the racemases and epimerases, or antigenic portions thereof, to induce a protective immune response against infection by parasites expressing those proteins. In addition, this invention relates to antibodies specific for the racemases and epimerases. Further, the invention relates to methods and kits for detecting racemases and epimerases using the nucleic acid molecules of the invention, as well as the peptides consisting of the motifs and antibodies to these peptides.

D-amino acids have long been described in the cell wall of gram-positive and especially gram-negative bacteria, where they constitute essential elements of the peptidoglycan and as substitutes of cell wall techoic acids (1). Moreover, various types of D-amino acids were discovered in a number of small peptides made by a variety of microorganisms through non-ribosomal protein synthesis (2), that function mainly as antibiotic agents. However, these examples were considered exceptions to the rule of homochirality and a dogma persisted that only L-amino acid enantiomers were present in eukaryotes, apart from a very low level of D-amino acids from spontaneous racemization due to aging (3).

Recently, an increasing number of studies have reported the presence of various D-amino acids (D-aa) either as protein bound (4) or free forms (5) in a wide variety of organisms, including mammals. The origin of free D-aa, is less clear than that of protein bound D-aa. For instance, in mammals, free D-aa may originate from exogenous sources (as described in (6)), but the recent discovery of amino acid racemases in eukaryotes has also uncovered an endogenous production of D-aa, questioning their specific functions. Thus, the level of D-aspartate is developmentally regulated in rat embryos (7), the binding of D-serine to NMDA mouse brain receptors promotes neuromodulation (8),(9), and D-aspartate appears to be involved in hormonal regulation in endocrine tissues (10).

Racemases and epimerases catalyze the deprotonation/reprotonation of the chiral carbon (C^{α}) of amino acid enantiomers resulting in stereoinversion of chiral centers. All amino acid racemases and epimerases require pyridoxal phosphate (PLP) as a cofactor, except proline, glutamate, and aspartate racemases, and hydroxyproline-2 and diaminopimelate epimerases, which are cofactor-independent enzymes. For example, two reports have been published addressing the biochemical and enzymatic characteristics of the proline racemase (PRAC) from the gram-positive bacterium Clostridium sticklandii (11,12). A reaction mechanism was proposed whereby the active site Cys²⁵⁶ forms a half-reaction site with the corresponding cysteine of the other monomer in the active, homodimeric enzyme.

Although a variety of racemases and epimerases have been demonstrated in bacteria and fungi, the first eukaryotic amino acid (proline) racemase isolated from the infective metacyclic form of the parasitic protozoan Trypanosoma cruzi, the causative agent of Chagas' disease in humans (13), was recently described. This parasite-secreted proline racemase (TcPRAC) was shown to be a potent mitogen for host B cells and to play an important role in T. cruzi immune evasion and persistence through polyclonal lymphocyte activation (13). This protein, previously annotated as TcPA45, with monomer size of 45 kDa, is only expressed and released by infective metacyclic forms of the parasite (13). TcPRAC is present in all T. cruzi life cycle stages, is essential for parasite viability, and appears to be involved in certain metabolic pathways during metacyclogenesis as parasites overexpressing TcPRAC genes gain better host infectivity.

Thermodynamic studies and the overall 3D-structure of homodimeric TcPRAC in complex with its competitive inhibitor provided evidence that Pro racemization operates by stabilization of carbanionic transition-state species in an acid/base catalytic mechanism (25). The genomic organization and transcription of TcPRAC proline racemase gene indicated the presence of two homologous genes per haploid genome (TcPRACA and TcPRACB). Furthermore, localization studies using specific antibodies directed to 45 kDa-TcPRAC protein revealed that an intracellular and/or membrane associated isoform, with monomer size of 39 kDa, is expressed in non-infective epimastigote forms of the parasite.

Computer-assisted analysis of the TcPRACA gene sequence suggested that it could give rise to both isoforms (45 kDa and 39 kDa) of parasite proline racemases through a mechanism of alternative trans-splicing, one of which would contain a signal peptide (13). In addition, preliminary analysis of putative TcPRACB gene sequences had revealed several differences that include point mutations as compared to TcPRACA, but that also suggest that the TcPRACB gene could only encode an intracellular isoform of the enzyme, as the gene lacks the export signal sequence. Any of these molecular mechanisms per se would ensure the differential expression of intracellular and extracellular isoforms of proline racemases produced in different T. cruzi developmental stages.

Amino acid racemases and epimerases are specific for their target amino acids. For example, hydroxyproline-2-epimerases (HyPRE) present overall sequence similarities with PRAC but react only with the C^{α} of 4-hydroxyproline (OH-Pro). In prokaryotic hosts, racemases are known to be implicated in the synthesis of D-amino acids and/or in the metabolism of L-amino acids. For instance, the presence of free D-amino acids in tumors and in progressive autoimmune and degenerative diseases suggests the biological importance of eukaryotic amino acid racemases. It is well known that proteins or peptides containing D-amino acids are resistant to proteolysis by host enzymes. In addition, proteins containing at least one D-amino acid residue can display antibiotic or immunogenic properties.

TcPRAC has been implicated in the regulation of intracellular proline metabolic pathways and post-translational addition of D-amino acids to polypeptide chains. HyPRE has been shown to be essential in P. putida, which, like other Pseudomonas spp., has been found to cause nosocomial infections with resulting septicemia and septic arthritis. Mutants lacking HyPRE are unable to metabolize OH-L-Pro and, hence, are not viable in OH-L-Pro-containing medium as the sole carbon source (37).

The human and animal pathogens that express PRACs and HyPREs affect multiple systems and result, for instance, in abscesses, pneumonia, and fatal septicemia in immunosuppressed hosts. For example, OH-L-Pro and L-Pro are the major constituents of collagen, the main component of the extracellular matrix, making up 25% of the total body protein content. Bacteria and viruses deprived of collagen have virulence factors, which destroy collagen or interfere with its production by the secretion of collagenase and/or elastase (38, 39). Bacterial meningitis, for example, can provoke collagen degradation and breakdown of the blood-brain barrier, which consequently raises bacterial invasiveness and persistence, resulting in brain injuries (40). Likewise, P. aeruginosa induces disruption of blood vessels through elastase by dissolution of the elastic lamina of arteries and arterioles, or by degrading major fragments of collagen IV (41).

Thus, there is a growing interest in the biological role of D-amino acids, either as free molecules or within polypeptide chains in human brain, tumors, anti-microbial and neuropeptides, suggesting widespread biological implications. However, research on D-amino acids in living organisms has been hampered by their difficult detection. There exists a need in the art for the identification of racemases and epimerases and the identification of their enzymatic properties and their specificity for other compounds.

Although much progress has been made concerning prophylaxis of Chagas' disease, particularly vector eradication, additional cases of infection and disease development still occur every day throughout the world. Whilst infection was largely limited in the past to vector transmission in endemic areas of Latin America, its impact has increased in terms of congenital and blood transmission, transplants and recrudescence following immunosuppressive states. Prevalence of Chagas' disease in Latin America may reach 25% of the population, as is the case of Bolivia, or yet 1%, as observed in Mexico. From the 18-20 million people already infected with the parasite Trypanosoma cruzi, more than 60% live in Brazil and WHO estimates that 90 million individuals are at risk in South and Central America.

Some figures obtained from a recent census in the USA, for instance, revealed that the net immigration from Mexico is about 1000 people/day, and of those, 5-10 individuals are infected by Chagas' disease. The disease can lie dormant for 10-30 years and, as is the case with many other progressive chronic pathologies, it is characterized by being “asymptomatic”. Although in the 1990's, blood banks increased their appeals to Hispanics (50% of Bolivian blood is contaminated), panels of Food and Drug Administration (FDA) have recommended that all donated blood be screened for Chagas. Today, FDA has not yet approved an ‘accurate’ blood test to screen donor blood samples. This allegation seriously contrasts with the more than 30 available tests used in endemic countries. Additionally, recent reports on new insect vectors adapted to the parasite and domestic animals infected in more developed countries like the USA, and the distributional predictions based on Genetic Algorithm for Rule-set Prediction models indicate a potentially broad distribution for these species and suggest additional areas of risk beyond those previously reported, emphasizing the continuing worldwide public health issue.

To date, two drugs are particularly used to treat Trypanosoma cruzi infections. Nifurtimox (3-methyl-4-5′-nitrofurfurylidene-amino tetrahydro 4H-1,4-thiazine-1,1-dioxide), a nitrofurane from Bayer, known as Lampit, was the first drug to be used since 1967. After 1973, Benznidazol, a nitroimidazol derivative, known as Rochagan or Radanyl (N-benzyl-2-nitro-1-imidazol acetamide) was produced by Hoffman-La-Roche and is consensually the drug of choice. Both drugs are trypanosomicides and act against intracellular or extracellular forms of the parasite. Adverse side-effects include a localized or generalized allergic dermopathy, peripheral sensitive polyneuropathy, leucopenia, anorexia, digestive manifestations and rare cases of convulsions which are reversible by interruption of treatment. The most serious complications include agranulocytosis and trombocytopenic purpura.

Unquestionably, the treatment is efficient and should be applied in acute phases of infection, in children, and in cases where reactivation of parasitaemia is observed following therapy with immunosuppressive drugs or organ transplantation procedures. Some experts recommend that patients in indeterminate and chronic phases should also be treated. However, close to a hundred years after the discovery of the infection and its consequent disease, researchers still maintain divergent points of view concerning therapy against the chronic phases of the disease. As one of the criteria of cure is based on the absence of the parasite in the blood, it is very difficult to evaluate the efficacy of the treatment in indeterminate or chronic phases. Because the indeterminate form is asymptomatic, it is impossible to clinically evaluate the cure. Furthermore, a combination of serology and more sensitive advanced molecular techniques will be required and still may not be conclusive. The follow-up of patients for many years is then inevitable to objectively ascertain the cure.

Chagas' disease was recently considered as a neglected disease and DND-initiative (Drug for Neglected Diseases Initiative, DNDi) wishes to support drug discovery projects focused on the development of effective, safe and affordable new drugs against trypanosomiasis. Since current therapies remain a matter of debate, may be inadequate in some circumstances, are rather toxic, and may be of limited effectiveness, the characterization of new formulations and the discovery of parasite molecules capable of eliciting protective immunity are absolutely required and must be considered as priorities.

### SUMMARY OF THE INVENTION

This invention aids in fulfilling these needs in the art. More particularly, this invention relates to the characterization of microorganism, especially parasite and bacterial, molecules implicated in polyclonal responses that may serve as novel targets for vaccination therapy. Using previously identified proline racemase (PRAC) signatures and data mining, this invention provides two novel PRACs and five novel hydroxyproline epimerases (HyPRE) from pathogenic bacteria.

In particular, this invention provides purified polypeptides comprising the amino acid sequences of SEQ ID NO: 131, SEQ ID NO: 133, SEQ ID NO: 135, SEQ ID NO: 137, SEQ ID NO: 139, SEQ ID NO: 141, and SEQ ID NO: 143 and antigenic polypeptide fragments thereof. Furthermore, enzymatic activities of parasite or bacterial PRAC and HyPREs are characterized and specific Vₘₐₓ and Kₘ are provided.

This invention additionally provides purified polynucleotides comprising the nucleic acid sequences of SEQ ID NO: 132, SEQ ID NO: 134, SEQ ID NO: 136, SEQ ID NO: 138, SEQ ID NO: 140, SEQ ID NO: 142, and SEQ ID NO: 144. In one embodiment of the invention, a recombinant DNA sequence comprising at least one of the nucleotide sequences enumerated above and under the control of regulatory elements that regulate the expression of racemase or epimerase activity in a host is provided.

In still a further embodiment of the invention, a method of detecting microorganism strains that contain the polynucleotide sequences set forth above is provided.

Additionally, the invention includes kits for the detection of the presence of microorganism strains that contain the polynucleotide sequences set forth above.

The invention also contemplates antibodies against the PRAC and HyPRE enzymes enumerated above, or antigenic portions thereof. This invention also relates to compositions comprising said antibodies. This invention also provides an immunizing composition containing at least a purified protein, or an antigenic fragment thereof, capable of inducing an immune response in vivo. The immune response can be a mitogenic polyclonal immunoresponse. The immunizing composition is suitable for use against a microorganism infection under sub-mitogenic doses.

This invention also provides a process to access the mitogenicity of a molecule called mitogen and the procedures to determine the sub-mitogenic dose suitable as an immunizing composition for use against a microorganism infection.

A method of inhibiting a eukaryotic or prokaryotic protein with an amino acid racemase or epimerase activity according to the invention comprises treating a patient by administering an effective amount of a molecule that inhibits the eukaryotic or prokaryotic protein.

This invention also provides a process for screening a molecule capable of inhibiting the amino acid racemase or epimerase activity of a eukaryotic or prokaryotic protein comprising the steps of: contacting the purified eukaryotic or prokaryotic racemase or epimerase protein with standard doses of a molecule to be tested; measuring inhibition of racemase or epimerase activity; and selecting the molecule.

This invention also provides nucleic acid and amino acid elements for in silico discrimination of PRAC and HyPRE enzymes. This invention further provides critical amino acid residues that are important in the enzymatic activity of hydroxyproline epimerases.

It has also been discovered that the TcPRAC genes in T. cruzi encode functional intracellular or secreted versions of the enzyme exhibiting distinct kinetic properties that may be relevant for their relative catalytic efficiency. While the K_{M} of the enzyme isoforms were of a similar order of magnitude (29-75 mM), Vₘₐₓ varied between 2×10⁻⁴ to 5.3×10⁻⁵ mol of L-proline/sec/0.125 μM of homodimeric recombinant protein. Studies with the enzyme specific inhibitor and abrogation of enzymatic activity by site-directed mutagenesis of the active site Cys³³⁰ residue reinforced the potential of proline racemase as a critical target for drug development against Chagas' disease.

This invention further provides a purified nucleic acid molecule encoding a peptide consisting of a motif selected from SEQ ID NOS: 1, 2, 3, 4, or 130.

This invention also provides a purified nucleic acid molecule that hybridizes to either strand of a denatured, double-stranded DNA comprising this nucleic acid molecule under conditions of moderate stringency.

In addition, this invention provides a recombinant vector that directs the expression of a nucleic acid molecule selected from these purified nucleic acid molecules.

The invention also includes a recombinant host cell comprising a polynucleotide sequence enumerated above or the recombinant vector defined above.

Further, this invention provides a purified polypeptide encoded by a nucleic acid molecule selected from the group consisting of a purified nucleic acid molecule coding for:

(a) a purified polypeptide consisting of Motif I (SEQ ID NO:1);

(b) a purified polypeptide consisting of Motif II (SEQ ID NO:2);

(c) a purified polypeptide consisting of Motif III (SEQ ID NO:3);

(d) a purified polypeptide consisting of Motif III*(SEQ ID NO:4); and

(e) a purified polypeptide consisting of R3 (SEQ ID NO:130).

Purified antibodies that bind to these polypeptides are provided. The purified antibodies can be monoclonal antibodies. An immunological complex comprises a polypeptide and an antibody that specifically recognizes the polypeptide of the invention.

A host cell transfected or transduced with the recombinant vector of the invention is provided.

A method for the production of a polypeptide consisting of SEQ ID NOS: 1, 2, 3, 4, or 130 comprises culturing a host cell of the invention under conditions promoting expression, and recovering the polypeptide from the host cell or the culture medium. The host cell can be a bacterial cell, parasite cell, or eukaryotic cell.

A method of the invention for detecting a racemase or epimerase encoded by a nucleotide sequence containing a subsequence encoding a peptide selected from SEQ ID NO: 1, 2, 3, 4, or 130 comprises:
(a) contacting the nucleotide sequence with a primer or a probe, which hybridizes with the nucleic acid molecule of the invention; and
(b) detecting a hybridized complex formed between the primer or probe and the nucleotide sequence.

Another method of the invention for detecting a racemase or epimerase encoded by a nucleotide sequence containing a subsequence encoding a peptide selected from SEQ ID NO: 1, 2, 3, 4, or 130, comprises:
(a) contacting the nucleotide sequence with a primer or a probe which hybridizes with the nucleic acid sequence;
(b) amplifying the nucleotide sequence using the primer or probe;
(c) contacting the amplified sequence with a primer or a probe which hybridizes with the nucleic acid molecule of the invention; and
(d) detecting a hybridized complex formed between the primer or probe and the amplified nucleotide sequence.

This invention provides a method of detecting a racemase or epimerase encoded by a nucleotide sequence containing a subsequence encoding a peptide selected from SEQ ID NO: 1, 2, 3, 4 or 130. The method comprises:
(a) contacting the racemase or epimerase with antibodies of the invention; and
(b) detecting the resulting immunocomplex.

This invention also provides a method of detecting antibodies directed against a racemase or epimerase using the polypeptide of the invention. The method comprises:
(a) contacting the antibodies with the polypeptide of the invention; and
(b) detecting the resulting immunocomplex.

A kit for detecting antibodies directed against a racemase or epimerase is contemplated by the invention. The kit comprises:
(a) a purified polypeptide of the invention;
(b) standard reagents in purified form; and
(d) detection reagents.

A kit for detecting a racemase or epimerase encoded by a nucleotide sequence containing a subsequence encoding a peptide selected from SEQ ID NO: 1, 2, 3, 4, or 130 comprises:
(a) a polynucleotide probe or primer, which hybridizes with the polynucleotide of the invention;
(b) optionally, a polynucleotide probe or primer, which hybridizes with the nucleotide sequence; and
(b) reagents to perform a nucleic acid hybridization reaction.

This invention also provides a kit for detecting a racemase or epimerase encoded by a nucleotide sequence containing a subsequence encoding a peptide selected from SEQ ID NO: 1, 2, 3, 4, or 130. The kit comprises:
(a) purified antibodies of the invention;
(b) standard reagents in a purified form; and
(c) detection reagents.

An in vitro method of screening for an active molecule capable of inhibiting a racemase or epimerase encoded by a nucleotide sequence containing a subsequence encoding a peptide selected from SEQ ID NO: 1, 2, 3, 4, or 130 comprises:
(a) contacting the active molecule with the racemase or epimerase;
(b) testing the capacity of the active molecule, at various concentrations, to inhibit the activity of the racemase or epimerase; and
(c) choosing the active molecule that provides an inhibitory effect of at least 80% on the activity of any racemase or epimerase.

In a preferred embodiment of the invention the racemase is a proline racemase and the epimerase is a hydroxyproline-2-epimerase.

The invention provides a method of stimulating a protective immune response against Clostridium difficile, Tripanosoma viviax, Pseudomonas aeruginosa, Burkholderia pseudomallei, Brucella abortus, Brucella melitensis, or Brucella suis in a patient by administering to the patient a purified polypeptide of the invention, or fragment thereof, capable of inducing an immune response.

Accordingly, an immunizing composition of the invention contains at least a purified polypeptide of the invention or a fragment thereof, capable of inducing an immune response in vivo, and a pharmaceutical carrier.

The invention also provides a method of stimulating a protective immune response against Clostridium difficile, Tripanosoma viviax, Pseudomonas aeruginosa, Burkholderia pseudomallei, Brucella abortus, Brucella melitensis, or Brucella suis in a patient by administering to the patient a purified nucleic acid encoding a polypeptide of the invention, or a fragment thereof, which is capable of inducing an immune response.

Accordingly, an immunizing composition of the invention contains at least a purified nucleic acid encoding a polypeptide of the invention, or a fragment thereof, which is capable of inducing an immune response, and a pharmaceutical carrier.

The invention further provides a method of identifying a target amino acid sequence as a putative proline racemase, wherein the method comprises:
(A) providing an amino acid sequence database;
(B) performing a computer assisted search of the database to compare a known amino acid sequence of a proline racemase with sequences in the database;
(C) identifying a target amino acid sequence in the database having at least about 25% homology to the known proline racemase amino acid sequence;
   wherein the target sequence is a putative proline racemase if:
(1) the target sequence has the motif MIII* [SEQ ID NO: 4];
(2) the target sequence has the motif MCGH;
(3) the target sequence has two catalytic Cys residues corresponding to Cys₁₆₀ (or Cys₁₃₀, if the signal peptide is not present) and Cys₃₃₀ (or Cys₃₀₀, if the signal peptide is not present) of Trypanosoma cruzi racemase (TcPRAC) [SEQ ID NO:30]; and
(4) the target sequence has a phenylalanine residue corresponding to Phe₁₃₂ (or Phe₁₀₂, if the signal sequence is not present) (R1) of TcPRAC; and
(5) the target sequence has a Cys or Leu (R2) residue corresponding to Cys₃₀₀ (or Cys₂₇₀, if the signal peptide is not present) of TcPRAC.

The invention also provides a method for identifying a target amino acid sequence as a putative hydroxyproline epimerase, wherein the method comprises:
(A) providing an amino acid sequence database;
(B) performing a computer assisted search of the database to compare a known amino acid sequence of a proline racemase or of a hydroxyproline epimerase with sequences in the database;
(C) identifying a target amino acid sequence in the database having at least about 25% homology to the known proline racemase or hydroxyproline epimerase amino acid sequence;
   wherein the target sequence is a putative epimerase if:
(1) the target sequence has the motif MIII* [SEQ ID NO: 4];
(2) the target sequence has the motif MCGH;
(3) the target sequence has two catalytic Cys residues corresponding to Cys₁₆₀ (or Cys₁₃₀, if the signal sequence is not present) and Cys₃₃₀ (or Cys₃₀₀, if the signal sequence is not present) of Trypanosoma cruzi racemase (TcPRAC) [SEQ ID NO:30]; and
(4) the target sequence has a Ser or Val residue corresponding to Phe₁₃₂ (or Phe₁₀₂ if the signal sequence is not present) (R1) of TcPRAC; and
(5) the target sequence has a H is residue corresponding to Cys₃₀₀ (or Cys₂₇₀ if the signal sequence is not present) of TcPRAC; and
(6) the target sequence has the R3 motif [SEQ ID NO: 130], which is absent from TcPRAC.

In addition, the invention provides a method for the catalyzed conversion of one enantiomer to another enantiomer, wherein the method comprises:
(A) providing a proline racemase selected from EF495346 (CdPRAC, C. difficile VPI10463) [SEQ ID NO:141] and EF175213 (TVPRAC, T. vivax) [SEQ ID NO:143];
(B) reacting the proline racemase with a substrate for the racemase in the presence of a buffer and at a pH for steroinversion of chiral centers in the substrate to thereby form one or more of the enantiomers.

The invention further provides a method for the catalyzed epimerization of OH-L-Pro and OH-D-Pro of 4-hydroxyproline, wherein the method comprises:
(A) providing at least one epimerase selected from:
EF495341 (PaHyPRE, P. aeruginosa PAK) [SEQ ID NO: 131],
EF495342 (BmHyPRE, B. melitensis 16M) [SEQ ID NO: 133],
EF495343 (BsHyPRE, B. suis 1330) [SEQ ID NO: 135],
EF495344 (BaHyPRE, B. abortus 544) [SEQ ID NO: 137], and EF495345 (BpHyPRE, B. pseudomallei K96243) [SEQ ID NO: 139]; and
(B) reacting the epimerase with 4-hydroxyproline in the presence of a buffer and at a pH for OH-L/D-Pro epimerization.

The invention also provides a method of reducing the catalytic activity of an epimerase by mutating the epimerase. In one embodiment, the catalytic activity of the epimerase can be reduced by mutating at least one of the cysteine residues corresponding to Cys₈₈ and Cys 236 of PaHyPRE. In another embodiment, the catalytic activity of the epimerase can be reduced by mutating the Val or Ser residue corresponding to Phe₁₀₂ of TcPRAC.

In addition, the invention provides a method of detecting a substrate for a proline racemase, wherein the method comprises:
(A) providing a composition suspected of containing the substrate;
(B) contacting the composition with EF495346 (CdPRAC, C. difficile VPI10463) [SEQ ID NO: 141] or EF175213 (TvPRAC, T. vivax) [SEQ ID NO: 143]; and
(C) assaying the resulting mixture for L-Pro racemization, D-Pro racemization, or both.

The invention also provides a method for detecting a substrate for a hydroxyproline-2-epimerase, wherein the method comprises:
(A) providing a composition suspected of containing the substrate;
(B) contacting the composition with at least one epimerase selected from:
EF495341 (PaHyPRE, P. aeruginosa PAK) [SEQ ID NO: 131],
EF495342 (BmHyPRE, B. melitensis 16M) [SEQ ID NO: 133],
EF495343 (BsHyPRE, B. suis 1330) [SEQ ID NO: 135],
EF495344 (BaHyPRE, B. abortus 544) [SEQ ID NO: 137], and
EF495345 (BpHyPRE, B. pseudomallei K96243) [SEQ ID NO: 139]; and
(C) assaying the resulting mixture for OH-L-Pro epimerization, OH-D-Pro epimerization, or both.

In addition, the invention provides a method for inhibiting the growth of Clostridium difficile, Tripanosoma viviax, Pseudomonas aeruginosa, Burkholderia pseudomallei, Brucella abortus, Brucella melitensis, or Brucella suis in a patient infected therewith by administering to the patient an active molecule capable of inhibiting the proline racemase or hydroxyproline-2-epimerase enzymes expressed by those microorganisms.

The invention further provides a method for preventing Clostridium difficile, Tripanosoma viviax, Pseudomonas aeruginosa, Burkholderia pseudomallei, Brucella abortus, Brucella melitensis, or Brucella suis from evading host cell immunity by administering to a patient infected therewith an active molecule capable of inhibiting the proline racemase or hydroxyproline-2-epimerase enzymes expressed by those microorganisms.

The invention also provides a method for preventing mitogen-induced proliferation of resting lymphocytes in a patient infected with Clostridium difficile, Tripanosoma viviax, Pseudomonas aeruginosa, Burkholderia pseudomallei, Brucella abortus, Brucella melitensis, or Brucella suis by administering to the patient an active molecule capable of inhibiting the proline racemase or hydroxyproline-2-epimerase enzymes expressed by those microorganisms.

### BRIEF DESCRIPTION OF THE DRAWINGS

This invention will be understood with reference to the drawings in which:

FIG. 1: Comparative analysis of sequences of T. cruzi TcPRACA and TcPRACB proline racemase isoforms. A. Alignment of TcPRACA (Tc-A) and TcPRACB (Tc-B) nucleotide sequences: non coding sequences are shown in italics; trans-splicing signals are underlined and putative spliced leader acceptor sites are double-underlined; the region encoding the computer-predicted signal peptide is indicated by double-headed arrow; initiation of translation for TcPRACA and TcPRACB are shown by single-headed arrows; nucleotides shaded in light and dark grey, represent respectively silent mutations or point mutations; box, proline racemase active site; UUA triplets are underlined in bold and precede polyadenylation sites that are double-underlined. B. Schematic representation of amino acid sequence alignments of T. cruzi TcPRACA (Tc-A), TcPRACB (Tc-B) proline racemases. The common scale is in amino acid residue positions along the linear alignment and represent the initiation codons for TcPRACA and TcPRACB proteins, respectively; V represents an alternative TcPRACA putative initiation codon; Amino acid differences are indicated above and below the vertical lines and their positions in the sequence are shown in parenthesis. SP: signal peptide; the N-terminal domain of TcPRACA extends from positions 1 to 69; SPCGT: conserved active sites of TcPRACA and TcPRACB proline racemases; N-terminus and C-terminus are indicated for both proteins. C. Hydrophobicity profile of TcPRACA: dotted line depicts the cleavage site as predicted by Von Heijne's method (aa 31-32). D. Ethidium bromide-stained gel of chromosomal bands of T. cruzi CL Brener clone after separation by PFGE (lane 1) and Southern blot hybridization with TcPRAC probe (lane 2). The sizes (Mb) of chromosomal bands are indicated, as well as the region chromosome numbers in roman numerals.

FIG. 2: Biochemical characterization of T. cruzi proline racemase isoforms and substrate specificities. A. SDS-PAGE analysis of purified rTcPRACA (lane 1) and rTcPRACB (lane 2) recombinant proteins. A 8% polyacrylamide gel was stained with Coomassie blue. Right margin, molecular weights. B. Percent of racemization of L-proline, D-proline, L-hydroxy (OH) proline and D-hydroxy (OH) proline substrates by rTcPRACB (open bar) as compared to rTcPRACA (closed bar). Racemase activity was determined with 0.25 μM of each isoform of proline racemase and 40 mM substrate in sodium acetate buffer pH 6.0. C. Percent of racemization as a function of pH: Racemase assays were performed in buffer containing 0.2 M Tris-HCl (squares), sodium acetate (triangles) and sodium phosphate (circles), 40 mM L-proline and 0.25 μM of purified rTcPRACA (closed symbols) and rTcPRACB (open symbols). After 30 min at 37° C., the reaction was stopped by heat inactivation and freezing. D. 39 kDa intracellular isoform was isolated from soluble (Ese) extracts of non-infective epimastigote forms of the parasite. Western-blots of serial dilutions of the soluble suspension was compared to known amounts of rTcPRACB protein and used for protein quantitation using Quantity One® software. Racemase assays were performed in sodium acetate buffer pH6, using 40 mM L-proline and the equivalent depicted amounts of 39 kDa (ng) contained in Ese extract.

FIG. 3: Kinetic parameters of L-proline racemization catalyzed by rTcPRACA and rTcPRACB proline racemase isoforms. The progress of racemization reaction was monitored polarimetrically, as previously described (13). A. The determination of the linear part of the curve was performed at 37° C. in medium containing 0.2 M sodium acetate, pH 6.0; 0.25 μM purified enzyme and 40 mM L-proline. rTcPRACA reactions are represented by black squares and rTcPRACB reactions by white squares. B. Initial rate of racemase activity was assayed at 37° C. in medium containing 0.2 M sodium acetate, pH 6.0, 0.125 μM of rTcPRACA (solid squares) or rTcPRACB (open squares) purified enzymes and different concentrations of L-proline. Lineweaver-Burk double reciprocal plots were used to determine values for K_{M} and Vₘₐₓ where 1N is plotted in function of 1/[S] and the slope of the curve represents Kₘ/Vₘₐₓ. Values obtained were confirmed by using the Kaleïdagraph® program and Michaelis-Menten equation. The values are representative of six experiments with different enzyme preparations. C. Double reciprocal plot kinetics of 0.125 μM rTcPRACA proline racemase isoform in the presence (open squares) or absence (solid squares) of 6.7 μM PAC competitive inhibitor in function of L-proline concentration. For comparison: K^{M} reported for the proline racemase of C. sticklandii was 2.3 mM; kinetic assays using the native protein obtained from a soluble epimastigote fraction revealed a K_{M} of 10.7 mM and a Kᵢ of 1.15 μM.

FIG. 4: Size exclusion chromatography of rTcPRACA protein using a Superdex 75 column. Fractions were eluted by HPLC at pH 6.0, B2 and B4 peaks correspond to rTcPRACA dimer and monomer species respectively. B1 and B5 eluted fractions were reloaded into the column (bold, see inserts) using the same conditions and compared to previous elution profile (not bold).

FIG. 5: Site-directed mutagenesis of TcPRACA proline racemase. Schematic representation of the active site mutagenesis of proline racemase of TcPRACA gene.

FIG. 6: Sequence alignments of proteins (Clustal X) obtained by screening SWISS-PROT and TrEMBL databases using motifs I, II and III. Amino acids involved in MI, MII and MIII are shaded in dark grey and light grey figures the 13-14 unspecific amino acids involved in M II. SWISS-PROT accession numbers of the sequences are in Tables V and XII.

FIG. 7: Cladogram of protein sequences obtained by T-coffee alignment radial tree. See Tables V and XII for SWISS-PROT protein accession numbers.

FIG. 8: shows the percent of racemisation inhibition of different L-proline concentrations (ranging from 10-40 mM) using the D-AAO (D-AAO/L-) microtest as compared to conventional detection using a polarimeter (Pol/L-).

FIG. 9: shows the comparison of D-AAO/HRP reaction using D-Proline alone or an equimolar mixture of D- and L-Proline as standard.

FIG. 10: shows optical density at 490 nm as a function of D-proline concentration under the conditions provided in Example 16.

FIG. 11: is a Graph obtained with the serial dilutions of D-proline, as positive reaction control Obs: OD of wells (−) average of OD obtained from blank wells.

FIG. 12: shows the loss of the enzymatic activity of proline racemase after mutagenesis of the residue Cys¹⁶⁰ or the residue Cys³³⁰, which correspond to Cys¹³⁰ and Cys³⁰⁰, respectively, if the signal sequence is not present.

FIG. 13: shows the enzymatic activities of PRAC and HyPRE from different pathogens. Optimal reaction conditions consisted of 10 μg of the enzyme and 20 mM of substrate in specific buffers during 30 min at 37° C. (A) Percent of L- or D-Pro racemization in NaOAc, pH 6; (B) Percent of OH-L_Pro or OH-D_Pro epimerization in TE, pH 8. P. aeruginosa (SC) and B. cenocepacia (CT) recombinant proteins whose sequences lack one of the two Cys catalytic residues do not display any PRAC or HyPRE activities.

FIG. 14: shows PrpA of B. abortus (BaSeq1) is a hydroxyproline-2-epimerase. Reactions were performed with 3-10 μg of the enzyme and 40 mM of substrate in specific buffers during 1 h at 37° C. (A) Pro racemization reactions were performed in NaOAc, pH 6. (B) OH-Pro epimerization reactions were set up in parallel in TE, pH 9. Data from Spera et al. (28) was transposed to the Figure under shade and TcPRAC was used as a control; BaPrPA: purported ‘proline racemase protein A’; BaSeq1 was produced from PrpA-corresponding sequence 1 from B. abortus (Table I and FIG. 20). Percent of L- or D-Pro racemization (C) and percent of OH-L-Pro or OH-D-Pro epimerization (D) using specific buffers, 10 μg of the enzyme and 20 mM of substrate during 30 min at 37° C.

FIG. 15: shows pyrrole-2-carboxylic acid (PYC), the specific inhibitor of PRAC, is not an inhibitor of HyPRE. (A) Percent of L-Pro racemization or (B) OH-L_Pro epimerization in the absence (black bars) or in the presence (white bars) of 1 or 10 mM PYC. Reactions were performed at 37° C. for 30 min with 10 μg of the corresponding enzymes in NaOAc, pH 6 (PRAC reactions) or TE, pH 8 (HyPRE reactions) and 20 mM of substrate.

FIG. 16: shows the kinetic parameters of proline racemization and hydroxyproline epimerization. Progress of enzymatic activities was monitored polarimetrically, as described previously (2). Initial rates were plotted in function of [S] and kinetic parameters determined with the Kaleidagraph® program and the Michaelis-Menten equation. Maximum rate (Vₘₐₓ) and Michaelis-Menten constant (Kₘ) were obtained at 37° C. by incubation of 20 μg/ml of each recombinant protein with increasing concentrations of specific L- (closed circles) or D- (open squares) substrates. (A) PRAC activity is depicted for C. difficile; (B) HyPRE activity is depicted for P. aeruginosa; (C) Kₘ and Vₘₐₓ records of HyPRE reactions using L- or D-enantiomers were distinctively obtained with recombinant enzymes of B. abortus (BaHyPRE), B. melitensis (BmHyPRE), B. suis (BsHyPRE) and B. pseudomallei (BpHyPRE). TcPRACA: Kₘ of 29 mM and Vₘₐₓ of 5,3×10⁻⁵ M.sec⁻¹ and TcPRACB Kₘ of 75 mM and Vₘₐₓ of 2×10⁻⁴ M.sec⁻¹.

FIG. 17: shows detailed characteristics of proline racemase sequences versus hydroxyproline-2-epimerases. MCGH and MIII* PRAC motifs are shaded respectively in yellow and green. Catalytic Cys residues are colored in red. R1, R2 and R3 indicate critical compulsory differences allowing for the discrimination of PRAC and HyPRE. In the left margin, sequences corresponding to PRAC are underlined contrasting to HyPRE sequences (plain text). Residues involved in substrate specificity are shaded in green (PRAC) or blue (HyPRE). The proposed signature for HyPRE (squared) gathers an additional block of specific residues downstream PRAC MIII*. Sequences that do not meet those requirements and thus present unknown functions are in light gray.

MIII* (DRSPCGXGXXAXXA): minimal signature for putative proline racemases, containing the Cys330 as described in (2) (Note: residue Cys330 is equivalent to residue Cys330 described in (2) with the crystallographic data of PRAC).

*: MCGH: additional motif containing the residue Cys130 as described in (25).

R1: Invariable phenylalanine residue in PRAC (corresponding to Phe102 in TCRRACA, as described in (25).

R2: Invariable histidine residue in HyPRE (corresponding to Cys/Leu in PRACs and to residue His₂₁₀ in Pseudomonas aeruginosa HyPRE.

R3: Invariable block of residues in HyPRE corresponding to XLA residues downstream of the PRAC MIII* motif; proposed signature for HyPRE (DRSPCGXGXXAXXAXLA).

FIG. 18: shows that site-directed mutagenesis of key residues of PaHyPRE results in loss of enzymatic activity. (A) and (B) Percent of epimerization of OH-L-Pro and OH-D-Pro, respectively, exhibited by WT PaHyPRE or C88S, C236S, V60G and V60F point mutants.

FIG. 19: shows PRAC and HyPRE structural data, pocket constraints and evolution. (A) Ribbon model of TcPRAC (green, PDB: 1W61) and PaHyPRE (purple, PDB: 2AZP) subunits revealing the overall similarities of the 3D-structures. Cys catalytic residues (orange). (B) Close view of TcPRAC (left panel) and PaHyPRE (right panel) pockets. The two catalytic Cys residues of PRAC(C₁₃₀ and C₃₀₀) and of HyPRE (C₈₈ and C₂₃₆) are shown in the reaction center colored in orange sticks. Hydrophobic F₁₀₂ (green sticks) and aliphatic V₆₀ (blue sticks) residues are depicted respectively in PRAC and HyPRE reaction centers where Pro and OH-Pro were modeled. Polarity hindrance imposed by the aromatic PRAC F₁₀₂ residue and the solvent accessible area for the ligand made possible by HyPRE V₆₀ residue are shown. (C) Phylogram of PRAC and HyPRE aligned sequences showing the unrooted tree using H. influenzae DapE as uncontroversial outgroup. Bacterial and protozoa PRAC cluster together suggesting that divergence of PRAC and HyPRE took place before the separation of bacteria and eukaryotes.

FIG. 20: shows sequence alignments of Brucella abortus PrpA and PrpB virulence factors. The figure presents the sequence of BaSeq1 used in this study and derived by PCR amplification of DNA from B. abortus strain 544 using primers designed based on B. abortus 9-941 strain sequence. Both sequences illustrated in the alignments are 100% homologous to sequence BAB1_{—}1800 encoding PrpA virulence factor from strain 2308 of B. abortus. All the sequences possess MI, MII, MIII* (shaded in green) and MCGH motifs (shaded in yellow) described as minimal signatures for putative proline racemases (3, 26). Cysteine residues involved in catalysis are colored in red. Note that additional elements shaded in light blue are associated with HyPRE enzymes. BAB1_{—}0366 sequence, described as an inactive enzyme, does not possess all the essential elements for PRAC or HyPRE enzymatic activities.

FIG. 21: shows the inhibition of HyPRE reactions with alkylating agents. (A) Reactions of Pro racemization or (B) OH-Pro epimerization were done in absence (black bars) or in presence (white bars) of 1 or 25 mM of iodoacetate (IAA) or iodoacetamide (IAM), respectively. The reactions were performed using 10 μg of protein with 20 mM of substrate in optimal condition buffers during 30 min at 37° C.

FIG. 22: shows the strategy for PaHyPRE site specific mutagenesis. The strategy for PaHyPRE point mutations was developed based on the alignment of TcPRAC and PaHyPRE wild type (WT) sequences. Phe₁₀₂ (F₁₀₂), Cys₁₃₀ and Cys₃₀₀ residues of TcPRAC and Val₆₀, Cys₈₈ and Cys₂₃₆ residues of PaHyPRE are shown as reference marks. To verify the critical role of cysteine residues in PaHyPRE enzymatic activity two point mutants C88S and C236S were constructed by mutation of the cysteines into serine (S) residues. To verify the importance of Val₆₀ in ligand accessibility as well as its weight in conformational pocket constraints, PaHyPRE Val₆₀, corresponding to the critical TcPRAC aromatic Phe₁₀₂ was mutated into glycine (G) or phenylalanine (F) to obtain V60G and V60F mutants, respectively.

### DETAILED DESCRIPTION OF THE INVENTION

Proline racemase catalyses the interconversion of L- and D-proline enantiomers and has to date been described in only two species. Originally found in the bacterium Clostridium sticklandii, it contains cysteine residues in the active site and does not require co-factors or other known coenzymes. The first eukaryotic amino acid (proline) racemase, after isolation and cloning of a gene from the pathogenic human parasite Trypanosoma cruzi, has been described. While this enzyme is intracellularly located in replicative non-infective forms of T. cruzi, membrane-bound and secreted forms of the enzyme are present upon differentiation of the parasite into non-dividing infective forms. The secreted isoform of proline racemase is a potent host B-cell mitogen supporting parasite evasion of specific immune responses.

Primarily it was essential to elucidate whether TcPRACB gene could encode a functional proline racemase. To answer this question, TcPRACA and TcPRACB paralogue genes were expressed in Escherichia coli and detailed studies were performed on biochemical and enzymatic characteristics of the recombinant proteins. This invention demonstrates that TcPRACB indeed encodes a functional proline racemase that exhibits slightly different kinetic parameters and biochemical characteristics when compared to TcPRACA enzyme. Enzymatic activities of the respective recombinant proteins showed that the 39 kDa intracellular isoform of proline racemase produced by TcPRACB construct is more stable and has higher rate of D/L-proline interconversion than the 45 kDa isoform produced by TcPRACA. Additionally, the dissociation constant of the enzyme-inhibitor complex (Kᵢ) obtained with pyrrole-2-carboxylic acid, the specific inhibitor of proline racemases, is lower for the recombinant TcPRACB enzyme.

Moreover, this invention demonstrates that Cys³³⁰ and Cys¹⁶⁰ are key amino acids of the proline racemase active site since the activity of the enzyme is totally abolished by site-direct mutagenesis of these residues. Also, multiple alignment of proline racemase amino acid sequences allowed the definition of protein signatures that can be used to identify putative proline racemases in other microorganisms. The significance of the presence of proline racemase in eukaryotes, particularly in T. cruzi, is discussed, as well as the consequences of this enzymatic activity in the biology and infectivity of the parasite.

This invention provides amino acid motifs, which are useful as signatures for proline racemases and hydroxyproline epimerases. These amino acid motifs are as follows:

MOTIF I

[SEQ ID NO:1]

[IVL][GD]XHXXG[ENM]XX[RD]X[VI]XG
MOTIF II

[SEQ ID NO:2]

[NSM][VA][EP][AS][FY]X(13, 14)[GK]X[IVL]XXD[IV]
[AS][YWF]GGX[FWY]
MOTIF III

[SEQ ID NO:3′]

DRSPXGXGXXAXXA
MOTIF III*

[SEQ ID NO:4]

DRSPCGXGXXAXXA

where “X” is an amino acid in each of these sequences.

This invention also provides polynucleotides encoding amino acid motifs, which are also referred to herein as the “polynucleotides of the invention” and the “polypeptides of the invention.”

Databases were screened using these polynucleotide or polypeptide sequences of TcPRACA. Motifs I to III were searched. M I corresponds to [IVL][GD]XHXXG[ENM]XX[RD]X[VI]XXG, M II to of [NSM][VA][EP][AS][FY]X(13,14)[GK]X[IVL]XXD[IV][AS][YWF] GGX[FWY] M III to DRSPXGXGXXAXXA and M III* to DRSPCGXGXXAXXA. Sequences presented in the annexes, where the conserved regions of 2 cysteine residues of the active site are squared, are presented in Table VI in bold with corresponding Accession numbers. The two cysteine residues are Cys³³⁰ and its homologue Cys¹⁶⁰, where residue Cys¹⁶⁰ mutation by a serine by site directed mutagenesis also induces a drastic loss of the enzymatic activity as for residue Cys³³⁰.

Proline racemase, an enzyme previously only described in protobacterium Clostridium sticklandii (11), was shown to be encoded also by the eukaryote Trypanosoma cruzi, a highly pathogenic protozoan parasite (13). The Trypanosoma cruzi proline racemase (TcPRAC), formerly called TcPA45, is an efficient mitogen for host B cells and is secreted by the metacyclic forms of the parasite upon infection, contributing to its immune-evasion and persistence through non-specific polyclonal lymphocyte activation (13). Previous results suggested that TcPRAC is encoded by two paralogous genes per haploid genome. Protein localization studies have also indicated that T. cruzi can differentially express intracellular and secreted versions of TcPRAC during cell cycle and differentiation, as the protein is found in the cytoplasm of non-infective replicative (epimastigote) forms of the parasite, and bound to the membrane or secreted in the infective, non-replicative (metacyclic trypomastigote) parasites (13).

This invention characterizes the two TcPRAC paralogues and demonstrates that both TcPRACA and TcPRACB give rise to functional isoforms of co-factor independent proline racemases, which display different biochemical properties that may well have important implications in the efficiency of the respective enzymatic activities. This invention shows that TcPRAC isoforms are highly stable and have the capacity to perform their activities across a large spectrum of pH. In addition, the affinity of pyrrol-carboxylic acid, a specific inhibitor of proline racemase, is higher for TcPRAC enzymes than for CsPR.

As suggested before by biochemical and theoretical studies for the bacterial proline racemase (11,17,18), TcPRAC activities rely on two monomeric enzyme subunits that perform interconversion of L- and/or D-proline enantiomers by a two base mechanism reaction in which the enzyme removes an α-hydrogen from the substrate and donates a proton to the opposite side of the α-carbon. It has been predicted that each subunit of the homodimer furnishes one of the sulphydryl groups (18).

The present invention demonstrates that TcPRAC enzymatic activities are bona fide dependent on the Cys³³⁰ residue of the active site, as site-specific ³³⁰Cys to Ser mutation totally abrogates L- and D-proline racemization, in agreement with a previous demonstration that TcPRAC enzymatic activity is abolished through alkylation with iodoacetate or iodoacetamide (13), similarly to the Clostridium proline racemase, where carboxymethylation was shown to occur specifically with the two cysteines of the reactive site leading to enzyme inactivation (12). The present invention demonstrates also that the residue Cys¹⁶⁰ is also a critical residue of the active site and that TcPRAC possesses two active sites in its homodimer. These observations make it possible to search for inhibitors by means of assays based on the native and mutated sequences.

While gene sequence analysis predicted that, by a mechanism of alternative splicing, TcPRACA could generate both intracellular and secreted versions of parasite proline racemase, the present invention demonstrates that TcPRACB gene sequence per se codes for a protein lacking the amino acids involved in peptide signal formation and an extra N-terminal domain present in TcPRACA protein, resembling more closely the CsPR. Thus, TcPRACB can only generate an intracellular version of TcPRAC proline racemase. This discovery makes it possible to carry out a search of one putative inhibitor of an intracellular enzyme that should penetrate the parasite cell.

Interestingly, the presence of two homologous copies of TcPRAC genes in the T. cruzi genome, coding for two similar polypeptides but with distinct specific biochemical properties, could reflect an evolutionary mechanism of gene duplication and a parasite strategy to ensure a better environmental flexibility. This assumption is supported by the potential of the TcPRACA gene to generate two related protein isoforms by alternative splicing, a mechanism that is particularly useful for cells that must respond rapidly to environmental stimuli. Primarily, trans-splicing appears to be an ancient process that may constitute a selective advantage for split genes in higher organisms (19) and alternative trans-splicing was only recently proven to occur in T. cruzi (20). As an alternative for promoter selection, the regulated production of intracellular and/or secreted isoforms of proline racemase in T. cruzi by alternative trans-splicing of TcPRACA gene would allow the stringent conservation of a constant protein domain and/or the possibility of acquisition of an additional secretory region domain. As a matter of fact, recent investigations using RT-PCR based strategy and a common 3′ probe to TcPRACA and TcPRACB sequences combined to a 5′ spliced leader oligonucleotide followed by cloning and sequencing of the resulting fragments have indeed proved that an intracellular version of TcPRAC may also originate from the TcPRACA gene, corroborating this hypothesis.

Gene duplication is a relatively common event in T. cruzi that adds complexity to parasite genomic studies. Moreover, TcPRAC chromosomal mapping revealed two chromosomal bands that possess more than 3 chromosomes each and that may indicate that proline racemase genes are mapped in size-polymorphic homologous chromosomes, an important finding for proline racemase gene family characterization. Preliminary results have, for instance, revealed that T. cruzi DM28c type I strain maps proline racemase genes to the same chromoblot regions identified with T. cruzi CL type II strain used in the present invention.

It is well known that proline constitutes an important source of energy for several organisms, such as several hemoflagellates (21), (22), (23), and for flight muscles in insects (24). Furthermore, a proline oxidase system was suggested in trypanosomes (25) and the studies reporting the abundance of proline in triatominae guts (26) have implicated proline in metabolic pathways of Trypanosoma cruzi parasites as well as in its differentiation in the digestive tract of the insect vector (27). Thus, it is well accepted that T. cruzi can use L-proline as a principal source of carbon (25).

Moreover, preliminary results using parasites cultured in defined media indicate that both epimastigotes, found in the vector, and infective metacyclic trypomastigote forms can efficiently metabolize L- or D-proline as the sole source of carbon. While certain reports indicate that biosynthesis of proline occurs in trypanosomes, i.e. via reduction of glutamate carbon chains or transamination reactions, an additional and direct physiological regulation of proline might exist in the parasite to control amino acid oxidation and its subsequent degradation or yet to allow proline utilization. In fact, a recent report showed two active proline transporter systems in T. cruzi (28). T. cruzi proline racemase may possibly play a consequential role in the regulation of intracellular proline metabolic pathways, or else, it could participate in mechanisms of post-translational addition of D-amino acid to polypeptide chains. In addition, OH-L-Pro upregulates expression of bacterial genes whose products are involved in vital metabolic pathways, such as OH-D-Pro oxidase, deaminase, and dehydrogenase (37).

On one hand, these hypotheses would allow for an energy gain and, on the other hand, would permit the parasite to evade host responses. In this respect, it was reported that a single D-amino acid addition in the N-terminus of a protein is sufficient to confer general resistance to lytic reactions involving host proteolytic enzymes (29). The expression of proteins containing D-amino acids in the parasite membrane would benefit the parasite inside host cell lysosomes, in addition to contributing to the initiation of polyclonal activation, as already described for polymers composed of D-enantiomers (30), (31). Although D-amino acid inclusion in T. cruzi proteins would benefit the parasite, this hypothesis remains to be proven and direct evidence is technically difficult to obtain.

PRAC enzymes have been described as being involved in evasion mechanisms of parasite and bacterial species through the induction of non-specific hypergammaglobulinemia and by the secretion of pleiotropic cytokines (1, 34). It is also worth noting that metacyclogenesis of epimastigotes into infective metacyclic forms involves parasite morphologic changes that include the migration of the kinetoplast, a structure that is physically linked to the parasite flagellum, and many other significant metabolic alterations that combine to confer infectivity/virulence to the parasite (13,32). Proline racemase was shown to be preferentially localized in the flagellar pocket of infective parasite forms after metacyclogenesis (13), as are many other known proteins secreted and involved in early infection (33).

It is also conceivable that parasite proline racemase may function as an early mediator for T. cruzi differentiation through intracellular modification of internalized environmental free proline, as suggested above and already observed in some bacterial systems. As an illustration, exogenous alanine has been described as playing an important role in bacterial transcriptional regulation by controlling an operon formed by genes coding for alanine racemase and a smaller subunit of bacterial dehydrogenase (34).

In bacteria, membrane alanine receptors are responsible for alanine and proline entry into the bacterial cell (35). It can then be hypothesized that the availability of proline in the insect gut milieu is associated with a mechanism of environmental sensing by specific receptors in the parasite membrane and would allow for parasite proline uptake and its further intracellular racemization. Proline racemase would then play a fundamental role in the regulation of parasite growth and differentiation by its participation in both metabolic energetic pathways and the expression of proteins containing D-proline, as described above, consequently conferring parasite infectivity and its ability to escape host specific responses.

Thus far, and contrasting to the intracellular isoform of TcPRAC found in epimastigote forms of T. cruzi, the ability of metacyclic and bloodstream forms of the parasite to express and secrete proline racemase may have further implications in hostparasite interaction. In fact, the parasite-secreted isoform of proline racemase participates actively in the induction of non-specific polyclonal B-cell responses upon host infection (13) and favors parasite evasion, thus ensuring its persistence in the host.

As described for other mitogens and parasite antigens (36), (37), (38), and in addition to its mitogenic property, TcPRAC could also be involved in modifications of host cell targets enabling better parasite attachment to host cell membranes in turn assuring improved infectivity. Since several reports associate accumulation of L-proline with muscular dysfunction (39) and inhibition of muscle contraction (40), the release of proline racemase by intracellular parasites could alternatively contribute to the maintenance of infection through regulation of L-proline concentration inside host cells, as proline was described as essential for the integrity of muscular cell targets. Therefore, it has recently been demonstrated that transgenic parasites hyperexpressing TcPRACA or TcPRACB genes, but not functional knock outs, are 5-10 times more infective to host target cells pointing to a critical role of proline racemases in the infectious process. Likewise, previous reports demonstrated that genetic inactivation of Lysteria monocytogenes alanine racemase and D-amino acid oxidase genes abolishes bacterial pathogenicity, since the presence of D-alanine is required for the synthesis of the mucopeptide component of the cell wall that protects virtually all bacteria from the external milieu (41).

Present analysis using identified critical conserved residues in TcPRAC and C. sticklandii proline racemase genes and the screening of SWISS-PROT and TrEMBL databases led to the discovery of a putative minimal signature for proline racemases, DRSPXGX[GA]XXAXXA, and to confirm the presence of putative proteins in at least 10 distinct organisms. Screening of unfinished genome sequences showed highly homologous proline racemase candidate genes in an additional 8 organisms, amongst which are the fungus Aspergillus fumigatus and the bacteria Bacillus anthracis and Clostridium botulinum. This is of particular interest, since racemases, but not proline racemases, are widespread in bacteria and only recently described in more complex organisms such as T. cruzi, (42,43). These findings may possibly reflect cell adaptative responses to extracellular stimuli and uncover more general mechanisms for the regulation of gene expression by D-amino acids in eukaryotes. The finding of similar genes in human and mouse genome databases using less stringent signatures for proline racemase is striking. However, the absence of the crucial amino acid cysteine in the putative active site of those predicted proteins suggests a different functionality than that of a proline racemase.

A number of source databases are available that contain either nucleic acid sequences and/or amino acid sequences for use with the invention in identifying or determining PRACs and HyPREs. A number of different methods of performing such sequence searches are known in the art. The databases can be specific for a particular organism or a collection of organisms. For example, there are databases for the C. elegans, Arabadopsis. sp., M. genitalium, M. jannaschii, E. coli, H. influenzae, S. cerevisiae, and others. The sequence data of a known PRAC and/or HyPRE, such as TcPRAC, can be aligned to the sequences in the databases using algorithms designed to measure homology between two or more sequences.

Such sequence alignment methods include, for example, BLAST (Altschul et al., 1990), BLITZ (MPsrch) (Sturrock & Collins, 1993), and FASTA (Person & Lipman, 1988). A homologous sequence will be recognized based upon a threshold homology value. The threshold value may be predetermined, although this is not required. The threshold value can be based upon the particular polynucleotide length. A number of different procedures can be used to align sequences. Typically, Smith-Waterman or Needleman-Wunsch algorithms are used. However, faster procedures such as BLAST, FASTA, and PSI-BLAST can also be used.

For example, optimal alignment of sequences for aligning a comparison window may be conducted by the local homology algorithm of Smith (Smith and Waterman, Adv Appl Math, 1981; Smith and Waterman, J Teor Biol, 1981; Smith and Waterman, J Mol Biol, 1981; Smith et al, J Mol Evol, 1981), by the homology alignment algorithm of Needleman (Needleman and Wuncsch, 1970), by the search of similarity method of Pearson (Pearson and Lipman, 1988), by computerized implementations of these algorithms (GAP, BESTFIT, FASTA, and TFASTA in the Wisconsin Genetics Software Package Release 7.0, Genetics Computer Group, 575 Science Dr., Madison, Wis., or the Sequence Analysis Software Package of the Genetics Computer Group, University of Wisconsin, Madison, Wis.), or by inspection. The best alignment (i.e., resulting in the highest percentage of homology over the comparison window) can be generated by the various methods selected. The similarity of the two sequences can then be predicted.

Such software programs match similar sequences by assigning degrees of homology to various deletions, substitutions and other modifications. The terms “homology” and “identity” in the context of two or more nucleic acids or polypeptide sequences, refer to two or more sequences or subsequences that are the same or have a specified percentage of amino acid residues or nucleotides that are the same when compared and aligned for maximum correspondence over a comparison window or designated region as measured using any number of sequence comparison algorithms or by manual alignment and visual inspection.

For sequence comparison, typically one sequence acts as a reference sequence, to which database sequences are compared. When using a sequence comparison algorithm, subsequence coordinates are designated, if necessary, and sequence algorithm program parameters are designated. Default program parameters can be used, or alternative parameters can be designated. The sequence comparison algorithm then calculates the percent sequence identities for the database sequences relative to the reference sequence, based on the program parameters.

A “comparison window”, as used herein, includes reference to a segment of any one of the number of contiguous positions selected from the group consisting of 20 to 600, usually about 50 to about 200, more usually about 100 to about 150 in which a sequence may be compared to a reference sequence of the same number of contiguous positions after the two sequences are optimally aligned.

One example of a useful algorithm is BLAST and BLAST 2.0 algorithms, which are described in Altschul et al., Nuc. Acids Res. 25:3389-3402 (1977) and Altschul et al., J. Mol. Biol. 215:403-410 (1990), respectively. Software for performing BLAST analyses is publicly available through the National Center for Biotechnology Information (www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0). The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4 and a comparison of both strands.

The BLAST algorithm also performs a statistical analysis of the similarity between two sequences (see, e.g., Karlin & Altschul, Proc. Natl. Acad. Sci. USA 90:5873 (1993)). One measure of similarity provided by BLAST algorithm is the smallest sum probability (P(N)), which provides an indication of the probability by which a match between two nucleotide sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

Sequence homology means that two polynucleotide sequences are homolgous (i.e., on a nucleotide-by-nucleotide basis) over the window of comparison. A percentage of sequence identity or homology is calculated by comparing two optimally aligned sequences over the window of comparison, determining the number of positions at which the identical nucleic acid base (e.g., A, T, C, G, U, or I) occurs in both sequences to yield the number of matched positions, dividing the number of matched positions by the total number of positions in the window of comparison (i.e., the window size), and multiplying the result by 100 to yield the percentage of sequence homology. This substantial homology denotes a characteristic of a polynucleotide sequence, wherein the polynucleotide comprises a sequence having at least 60 percent sequence homology, typically at least 70 percent homology, often 80 to 90 percent sequence homology, and most commonly at least 99 percent sequence homology as compared to a reference sequence of a comparison window of at least 25-50 nucleotides, wherein the percentage of sequence homology is calculated by comparing the reference sequence to the polynucleotide sequence which may include deletions or additions which total 20 percent or less of the reference sequence over the window of comparison.

Once a gene is sequenced, the present invention provides a means to compare alleles or related sequences to locate and identify differences from the control sequence. This would be extremely useful in further analysis of genetic variability at a specific gene locus or for distinguishing between PRACs and HyPREs. The sequence analysis may be performed with polynucleotide sequences or polypeptide sequences.

Using previously identified PRAC signatures and data mining, PRAC homologues from pathogens were investigated by screening released genomic databases to further explore novel potential therapeutic targets. When the MIII* signature was used for screening, 111 hits were obtained, 92 of which possessed both catalytic cysteine residues.

The presence of functional PRAC was investigated in a collection of 9 bacterial species of pathogenic importance (i.e. Firmicute, α-, β- and γ-proteobacteria) using molecular and biochemical approaches. This invention unveils two new functional PRACs isolated from Clostridium difficile and Trypanosoma vivax and 5 novel functional hydroxyproline-2-epimerases (HyPRE) from Pseudomonas aeruginosa, Burkholderia pseudomallei and 3 Brucella species. In addition, this invention reveals that a Brucella abortus virulence factor (PrpA), previously described as a PRAC (28), as well as homologous proteins from B. melitensis and B. suis, are PLP-independent HyPREs that interconvert trans or cis OH-L-Pro into cis or trans OH-D-Pro, respectively.

The invention also reveals that MIII* is not sufficiently stringent to discriminate PRACs from HyPREs. Additional element motifs are provided for the discrimination of PRAC and HyPRE sequences based, for instance, on polarity constraints imposed by precise residues of the catalytic pockets that contribute to ligand specificity. Those elements are as follows:
R1: An invariable phenylalanine residue in PRAC corresponding to Phe¹⁰² in TcPRACA)
R2: An invariable histidine residue in HyPRE corresponding to Cys/Leu in PRAC and to residue His²¹⁰ in Pseudomonas aeruginosa HyPRE
R3: DRSPCGXGXXAXXAXLA [SEQ ID NO:130] in HyPRE, where X is an amino acid.

Table I depicts the corresponding positions in different microorganisms of key catalytic residues (in block MCGH and MIII* motifs), R1, R2, and R3 discriminating elements between PRAC and HyPRE enzymes, as well as complementary (CLA) residues to the MIII* motif corresponding to the HyPRE signature (i.e., DRSPCGXGXXAXXAXLA). It is important to note that the full-length sequence for TcPRACA possesses a signal peptide (30aa) that allows the production of a secreted isoform of the enzyme. Crystallographic data was obtained from the soluble recombinant TcPRACA protein produced from a truncated gene sequence construct, i.e., lacking the hydrophobic signal peptide. Thus, Cys160 and Cys330 catalytic cysteine residues from the full length TcPRACA sequence described throughout the text, figures and tables may also correspond respectively to the cysteine residues at position 130 and 300 of the mature (soluble) protein. The TcPRACB sequence does not possess a signal for secretion and thus is an intracytoplasmic isoform of PRAC.

TABLE I

| Enzyme | Pathogen | Sequence | R1 | MCGH | R2 | MIII* | R3 | XLA |
|---|---|---|---|---|---|---|---|---|
| PRAC | Trypanosoma | TcPRACA | Phe₁₃₂ | Cys₁₆₀ | Cys₃₀₀ | Cys₃₃₀ | Tyr₃₄₂ | no |
| | cruzi | (with Signal | | | | | | |
| | | peptide) | | | | | | |
| PRAC | Trypanosoma | TcPRACA | Phe₁₀₂ | Cys₁₃₀ | Cys₂₇₀ | Cys₃₀₀ | Tyr₃₁₂ | no |
| | cruzi | (w/o Signal | | | | | | |
| | | peptide) | | | | | | |
| PRAC | Trypanosoma | TcPRACB | Phe₆₃ | Cys₉₁ | Cys₂₃₁ | Cys₂₆₁ | Tyr₂₇₃ | no |
| | cruzi | | | | | | | |
| PRAC | Trypanosoma | TvPRAC | Phe₆₃ | Cys₉₁ | Cys₂₃₂ | Cys₂₆₂ | Tyr₂₇₄ | no |
| | vivax | | | | | | | |
| PRAC | Clostridium | CdPRAC | Phe₆₃ | Cys₉₁ | Leu₂₂₆ | Cys₂₅₆ | His₂₆₈ | no |
| | difficile | | | | | | | |
| PRAC | Clostridium | CsPRAC | Phe₃₀ | Cys₅₈ | Leu₁₉₃ | Cys₂₂₃ | Tyr₂₃₅ | no |
| | sticklandii | | | | | | | |
| HyPRE | Brucella | BaHyPRE | Ser₆₂ | Cys₉₀ | His₂₂₄ | Cys₂₅₃ | Ala₂₆₅ | QLA |
| | abortus | | | | | | | |
| HyPRE | Brucella suis | BsHyPRE | Ser₆₂ | Cys₉₀ | His₂₂₄ | Cys₂₅₃ | Ala₂₆₅ | QLA |
| HyPRE | Brucella | BmHyPRE | Ser₆₂ | Cys₉₀ | His₂₂₄ | Cys₂₅₃ | Ala₂₆₅ | QLA |
| | melitensis | | | | | | | |
| HyPRE | Burkholderia | BpHyPRE | Val₆₀ | Cys₈₈ | His₂₁₀ | Cys₂₃₆ | Ala₂₄₈ | CLA |
| | pseudomallei | | | | | | | |
| HyPRE | Pseudomonas | PpHyPRE | Val₆₀ | Cys₈₈ | His₂₁₀ | Cys₂₃₆ | Ala₂₄₈ | CLA |
| | putida | | | | | | | |
| HyPRE | Pseudomonas | PfHyPRE | Val₆₀ | Cys₈₈ | His₂₁₀ | Cys₂₃₆ | Ala₂₄₈ | CLA |
| | fluorescens | | | | | | | |
| HyPRE | Pseudomonas | PaHyPRE | Val₆₀ | Cys₈₈ | His₂₁₀ | Cys₂₃₆ | Ala₂₄₈ | CLA |
| | aeruginosa | | | | | | | |

The enzymatic activities of the novel PRACs and HyPREs were fully characterized and specific Vₘₐₓ and Kₘ are provided. This invention reveals that HyPRE enzymatic activity, like PRAC activity, depends on two catalytic Cys. In addition, this invention identifies a critical Val residue in the enzymatic activity of HyPREs. Specifically, Cys⁸⁸, Cys²³⁶, and Val⁶⁰ are identified as being important in the enzymatic activity of HyPREs. Accordingly, this is the first disclosure associating full-length HyPRE genes and the functional enzymatic activity of the encoded proteins.

The invention also provides amino acid or nucleic acid sequences substantially similar to specific sequences disclosed herein.

The term “substantially similar” when used to define either amino acid or nucleic acid sequences means that a particular subject sequence, for example, a mutant sequence, varies from a reference sequence by one or more substitutions, deletions, or additions, the net effect of which is to retain activity. Alternatively, nucleic acid subunits and analogs are “substantially similar” to the specific DNA sequences disclosed herein if: (a) the DNA sequence is derived from a region of the invention; (b) the DNA sequence is capable of hybridization to DNA sequences of (a) and/or which encodes active PRACs or HyPREs; or DNA sequences that are degenerate as a result of the genetic code to the DNA sequences defined in (a) or (b) and/or which encode active PRACs or HyPREs.

In order to preserve the activity, deletions and substitutions will preferably result in homologously or conservatively substituted sequences, meaning that a given residue is replaced by a biologically similar residue. Examples of conservative substitutions include substitution of one aliphatic residue for another, such as Ile, Val, Leu, or Ala for one another, or substitution of one polar residue for another, such as between Lys and Arg; Glu and Asp; or Gln and Asn. Other such conservative substitutions, for example, substitutions of entire regions having similar hydrophobicity characteristics, are well known. When said activity is proline racemase activity, Cys³³⁰ and Cys¹⁶⁰ must be present. When said activity is hydroxyproline epimerase, Cys⁸⁸, Cys²³⁶, and Val⁶⁰ must be present.

The polynucleotides of the invention can be used as probes or to select nucleotide primers notably for an amplification reaction. PCR is described in the U.S. Pat. No. 4,683,202 granted to Cetus Corp. The amplified fragments can be identified by agarose or polyacrylamide gel electrophoresis, or by a capillary electrophoresis, or alternatively by a chromatography technique (gel filtration, hydrophobic chromatography, or ion exchange chromatography). The specificity of the amplification can be ensured by a molecular hybridization using as nucleic acid probes the polynucleotides of the invention, oligonucleotides that are complementary to these polynucleotides, or their amplification products themselves.

Amplified nucleotide fragments are useful as probes in hybridization reactions in order to detect the presence of one polynucleotide according to the present invention or in order to detect the presence of a gene encoding racemase or epimerase activity, such as in a biological sample. This invention also provides the amplified nucleic acid fragments (“amplicons”) defined herein above. These probes and amplicons can be radioactively or non-radioactively labeled using, for example, enzymes or fluorescent compounds.

Other techniques related to nucleic acid amplification can also be used alternatively to the PCR technique. The Strand Displacement Amplification (SDA) technique (Walker et al., 1992) is an isothermal amplification technique based on the ability of a restriction enzyme to cleave one of the strands at a recognition site (which is under a hemiphosphorothioate form), and on the property of a DNA polymerase to initiate the synthesis of a new strand from the 3′ OH end generated by the restriction enzyme, and on the property of this DNA polymerase to displace the previously synthesized strand being localized downstream.

The SDA amplification technique is more easily performed than PCR (a single thermostated water bath device is necessary), and is faster than the other amplification methods. Thus, the present invention also comprises using the nucleic acid fragments according to the invention (primers) in a method of DNA or RNA amplification, such as the SDA technique.

The polynucleotides of the invention, especially the primers according to the invention, are useful as technical means for performing different target nucleic acid amplification methods, such as:
TAS (Transcription-based Amplification System), described by Kwoh et al. in 1989;
SR (Self-Sustained Sequence Replication), described by Guatelli et al. in 1990;
NASBA (Nucleic Acid Sequence Based Amplification), described by Kievitis et al. in 1991; and
TMA (Transcription Mediated Amplification).

The polynucleotides of the invention, especially the primers according to the invention, are also useful as technical means for performing methods for amplification or modification of a nucleic acid used as a probe, such as:
LCR (Ligase Chain Reaction), described by Landegren et al. in 1988 and improved by Barany et al. in 1991, who employ a thermostable ligase;
RCR (Repair Chain Reaction), described by Segev et al. in 1992;
CPR (Cycling Probe Reaction), described by Duck et al. in 1990; and
Q-beta replicase reaction, described by Miele et al. in 1983 and improved by Chu et al. in 1986, Lizardi et al. in 1988, and by Burg et al. and Stone et al. in 1996.

When the target polynucleotide to be detected is RNA, for example mRNA, a reverse transcriptase enzyme can be used before the amplification reaction in order to obtain a cDNA from the RNA contained in the biological sample. The generated cDNA can be subsequently used as the nucleic acid target for the primers or the probes used in an amplification process or a detection process according to the present invention.

The oligonucleotide probes according to the present invention hybridize specifically with a DNA or RNA molecule comprising all or part of the polynucleotide of the invention under stringent conditions. As an illustrative embodiment, the stringent hybridization conditions used in order to specifically detect a polynucleotide according to the present invention are advantageously the following:

Prehybridization and hybridization are performed as follows in order to increase the probability for heterologous hybridization:
The prehybridization and hybridization are done at 50° C. in a solution containing 5×SSC and 1×Denhardt's solution.

The washings are performed as follows:
2×SSC at 60° C. 3 times during 20 minutes each.

The non-labeled polynucleotides of the invention can be directly used as probes. Nevertheless, the polynucleotides can generally be labeled with a radioactive element (³²P, ³⁵S, ³H, ¹²⁵I) or by a non-isotopic molecule (for example, biotin, acetylaminofluorene, digoxigenin, 5-bromodesoxyuridin, fluorescein) in order to generate probes that are useful for numerous applications. Examples of non-radioactive labeling of nucleic acid fragments are described in the French Patent No. FR 78 10975 or by Urdea et al. or Sanchez-Pescador et al. 1988.

Other labeling techniques can also be used, such as those described in the French patents 2 422 956 and 2 518 755. The hybridization step can be performed in different ways. A general method comprises immobilizing the nucleic acid that has been extracted from the biological sample on a substrate (nitrocellulose, nylon, polystyrene) and then incubating, in defined conditions, the target nucleic acid with the probe. Subsequent to the hybridization step, the excess amount of the specific probe is discarded, and the hybrid molecules formed are detected by an appropriate method (radioactivity, fluorescence, or enzyme activity measurement).

Advantageously, the probes according to the present invention can have structural characteristics such that they allow signal amplification, such structural characteristics being, for example, branched DNA probes as those described by Urdea et al. in 1991 or in the European Patent No. 0 225 807 (Chiron).

In another advantageous embodiment of the present invention, the probes described herein can be used as “capture probes”, and are for this purpose immobilized on a substrate in order to capture the target nucleic acid contained in a biological sample. The captured target nucleic acid is subsequently detected with a second probe, which recognizes a sequence of the target nucleic acid that is different from the sequence recognized by the capture probe.

A chemical method for producing the nucleic acids according to the invention comprises the following steps:
assembling the chemically synthesized oligonucleotides, which can have different restriction sites at each end;
cloning the thus obtained nucleic acids in an appropriate vector; and
purifying the nucleic acid by hybridizing to an appropriate probe according to the present invention.

The oligonucleotide probes according to the present invention can also be used in a detection device comprising a matrix library of probes immobilized on a substrate, the sequence of each probe of a given length being localized in a shift of one or several bases, one from the other, each probe of the matrix library thus being complementary to a distinct sequence of the target nucleic acid. Optionally, the substrate of the matrix can be a material able to act as an electron donor, the detection of the matrix positions in which hybridization has occurred being subsequently determined by an electronic device. Such matrix libraries of probes and methods of specific detection of a target nucleic acid are described in European patent application No. 0 713 016, or PCT Application No. WO 95 33846, or also PCT Application No. WO 95 11995 (Affymax Technologies), PCT Application No. WO 97 02357 (Affymetrix Inc.), and also in U.S. Pat. No. 5,202,231 (Drmanac), said patents and patent applications being herein incorporated by reference.

The present invention also pertains to recombinant plasmids containing at least a nucleic acid according to the invention. A suitable vector for the expression in bacteria, and in particular in E. coli, is pET-28 (Novagen), which allows the production of a recombinant protein containing a 6×His affinity tag. The 6×His tag is placed at the C-terminus or N-terminus of the recombinant polypeptide.

The polypeptides according to the invention can also be prepared by conventional methods of chemical synthesis, either in a homogenous solution or in solid phase. As an illustrative embodiment of such chemical polypeptide synthesis techniques, the homogenous solution technique described by Houbenweyl in 1974 may be cited.

The polypeptides of the invention are useful for the preparation of polyclonal or monoclonal antibodies. In particular, the invention relates to antibodies that recognize the polypeptides (SEQ ID NOS: 1, 2, 3, 4, and 130) or fragments thereof. The monoclonal antibodies can be prepared from hybridomas according to the technique described by Kohler and Milstein in 1975. The polyclonal antibodies can be prepared by immunization of a mammal, especially a mouse or a rabbit, with a polypeptide according to the invention, which is combined with an adjuvant, and then by purifying specific antibodies contained in the serum of the immunized animal on an affinity chromatography column on which has previously been immobilized the polypeptide that has been used as the antigen.

The invention also relates to a method of detecting a racemase or epimerase encoded by a nucleotide sequence containing a subsequence encoding a peptide selected from SEQ ID NOS: 1, 2, 3, 4, or 130.

Consequently, the invention is also directed to a method for detecting specifically the presence of a polypeptide according to the invention in a biological sample. The method comprises:
a) bringing into contact the biological sample with an antibody according to the invention; and
b) detecting antigen-antibody complex formed.

Also part of the invention is a diagnostic kit for in vitro detecting the presence of a polypeptide according to the present invention in a biological sample. The kit comprises:
a polyclonal or monoclonal antibody as described above, optionally labeled; and
a reagent allowing the detection of the antigen-antibody complexes formed, wherein the reagent carries optionally a label, or being able to be recognized itself by a labeled reagent, more particularly in the case when the above-mentioned monoclonal or polyclonal antibody is not labeled by itself.

The present invention is also directed to bioinformatic searches in data banks using Motifs I, II, III, III*, R1, R2, R3. The present invention also relates to bioinformatic searches in data banks using the whole sequences of the polypeptides corresponding to SEQ ID NOS: 1, 2, 3, 4, or 130. In this case the method detects the presence of at least a subsequence encoding a peptide selected from SEQ ID NOS: 1, 2, 3, 4, or 130 wherein the said at least subsequence is indicative of a racemase or epimerase.

The invention also pertains to:
A purified polypeptide or a peptide fragment having at least 10 amino acids, which is recognized by antibodies directed against a polynucleotide or peptide sequence according to the invention.
A monoclonal or polyclonal antibody directed against a polypeptide or a peptide fragment encoded by the polynucleotide sequences according to the invention.
A method of detecting the presence of a microorganism harboring a racemase or epimerase in a biological sample comprising:
   a) contacting DNA or RNA of the biological sample with a primer or a probe comprising a polynucleotide according to the invention, which hybridizes with a nucleotide sequence; and
   b) detecting the hybridized complex formed between the primer or probe with the DNA or RNA.
A method of detecting the presence of a microorganism harboring a racemase or epimerase in a biological sample comprising:
   a) contacting DNA or RNA of the biological sample with a primer or a probe which hybridizes to the sample;
   b) amplifying the nucleotide sequence using the primer or probe;
   c) contacting the amplified nucleotide sequence with a primer or a probe comprising a polynucleotide according to the invention, which hybridizes with a nucleotide sequence; and
   d) detecting the hybridized complex formed between the primer or probe with the amplified nucleotide sequence.

A kit for detecting the presence of a microorganism harboring a racemase or epimerase in a biological sample, comprises:
a) a polynucleotide primer or probe according to the invention; and
b) reagents necessary to perform a nucleic acid hybridization reaction.

A method of screening active molecules for the treatment of the infections due to a microorganism comprises the steps of:
a) bringing into contact a microorganism containing the polynucleotide sequences according to the invention with the molecule; and
b) measuring an activity of the active molecule on the microorganism.
   By “active molecule” according to the invention is meant a molecule capable of inhibiting the activity of a racemase or epimerase of the invention.

A test for screening the inhibiting activity of a molecule, for example, a new substrate analogue or a new antiparasitic, antibacterial, or antiviral agent, for inhibiting a PRAC or HyPRE can comprise the following steps:
The purified recombinant or native racemase or epimerase is diluted in sodium acetate buffer or Tris or phosphate buffer or Hepes buffer at 10 micrograms per 500 microliters in the presence of 20 millimolar of L or D substrate and containing various concentrations of active molecule to be tested. This reaction is incubated for 30 minutes at 37° C. and is stopped by heating at 80° C. Variations in optical rotation are measured by a polarimeter.

An in vitro method of screening for an active molecule capable of inhibiting a racemase or epimerase encoded by a nucleic acid containing a polynucleotide according to the invention, wherein the inhibiting activity of the molecule is tested on at least said racemase or epimerase, comprises:
a) providing a racemase or epimerase according to the invention;
b) contacting the active molecule with said racemase or epimerase;
c) testing the capacity of the active molecule, at various concentrations, to inhibit the activity of the racemase or epimerase; and
d) choosing the active molecule that provides an inhibitory effect of at least 80% on the activity of the racemase or epimerase.

Another embodiment of this invention provides a method for inhibiting the activity of a microorganism in vivo. The method can comprise administering to a host a purified PRAC or HyPRE, or antigenic fragment thereof, to stimulate a protective immune response against microorganisms expressing PRACs or HyPREs. The method can also comprise administering to a host antibodies against PRAC or HyPRE enzymes, wherein the antibodies block the activity of the PRAC or HyPRE enzymes. In addition, the method comprises administering to a host a microorganism mitogen. The antigens, antibodies, or mitogens are administered to the host in an amount sufficient to prevent or at least inhibit infection in vivo or to prevent or at least inhibit spread of the microorganism in vivo.

The parasite mitogen employed in this invention is distinguished from an “antigen,” which is a substance that induces an immune response, such as a complete antigen that both induces an immune response and reacts with the product of the response, or an incomplete antigen (hapten) that cannot induce an immune response by itself, but can react with the products of an immune response when complexed to a complete antigen (carrier). The parasite mitogens of the present invention are thus unlike antigens, which require processing and presentation, such as (1) uptake of the antigen by antigen presenting cells (APCs); (2) internalization of the antigen in intracellular vesicles; (3) intracellular processing, which may include the unfolding of a protein and/or partial proteolysis, with generation of immunogenic peptides; (4) binding of peptides to class II MHC molecules to form a bimolecular complex recognized by T cells; and (5) transport to, and display of, the complex on the surface of APCs. In addition, the parasite mitogens employed in this invention do not require activation of the APCs as manifested by the expression of: (1) adhesion molecules that promote the physical interaction between APCs and T cells; (2) membrane bound growth/differentiation molecules (co-stimulators) that promote T cell activation; or (3) soluble cytokines, such as IL-1 and TNF, as is required in the process for presenting antigens.

The mitogen employed in this invention is also distinguished from a “superantigen,” which is a substance that can stimulate all of the T cells in an individual that express a particular set or family of V_{β}T cell receptor genes. Superantigens are typically bacterial and viral products, and can either be soluble or cell-bound. They do not require degradation to peptides. Superantigens are typically presented to the T cell receptor (TCR) on MHC molecules; however, they do not require processing by antigen presenting cells (APC), as do antigens, in order to be presented.

Thus, used herein, the term “mitogen” refers to a polyclonal activator that has the capacity to bind to and to trigger proliferation or differentiation of B lymphocytes, T lymphocytes, or mixtures thereof. Lymphocyte proliferation or transformation is the process whereby new DNA synthesis and cell division takes place in lymphocytes after a stimulus of some type, resulting in a series of changes. The lymphocytes increase in size, the cytoplasm becomes more extensive, the nucleoli are visible in the nucleus, and the lymphocytes resemble blast cells. The term blast transformation is also sometimes applied to this process. Mitogens can induce proliferation in normal cells in culture. Activation of the lymphocytes thus can be characterized by transformation of the lymphocytes into blast cells, synthesis of DNA, cell division, increased production of immunoglobulins, or increased cytokine production. More particularly, the mitogens employed in this invention can stimulate whole classes of lymphocytes in this manner, and not just clones of particular specificity. The mitogens employed in this invention function, therefore, in a manner similar to the effects produced by lipopolysaccharide (LPS) on B cells, or lectins, concanavalin A (ConA), and phytohemagglutinin (PHA) on T cells.

With these phenomena in mind, the expression “microorganism mitogen,” as used herein, means at least one protein or polypeptide found in a microorganism, wherein the protein or polypeptide is capable of provoking non-specific polyclonal activation of B lymphocytes, T lymphocytes, or mixtures thereof, in an in vitro culture of the lymphocytes in the manner similar to that just described. The protein or polypeptide comprising the microorganism mitogen can be in glycosylated or non-glycosylated form. The microorganism mitogen can be in natural or recombinant form.

The term “recombinant” as used herein means that a protein or polypeptide employed in the invention is derived from recombinant (e.g., microbial or mammalian) expression systems. “Microbial” refers to recombinant proteins or polypeptides made in bacterial or fungal (e.g., yeast) expression systems. As a product, “recombinant microbial” defines a protein or polypeptide produced in a microbial expression system, which is essentially free of native endogenous substances. Proteins or polypeptides expressed in most bacterial cultures, e.g. E. coli, will be free of glycan. Proteins or polypeptides expressed in yeast may have a glycosylation pattern different from that expressed in mammalian cells.

The polypeptides or polynucleotides of this invention can be in isolated or purified form. The terms “isolated” or “purified”, as used in the context of this specification to define the purity of protein or polypeptide compositions, means that the protein or polypeptide composition is substantially free of other proteins of natural or endogenous origin and contains less than about 1% by mass of protein contaminants residual of production processes. Such compositions, however, can contain other proteins added as stabilizers, excipients, or co-therapeutics. These properties similarly apply to polynucleotides of the invention.

Evaluation of lymphocyte proliferation can be quantitated in an assay of proliferative activity. For example, a radiolabelled precursor of DNA (usually tritiated thymidine) can be added to a culture medium and the amount of radioactivity incorporated into the cells subsequently detected. A suitable assay involves the in vitro culture of a lymphocyte population in the presence or absence of a mitogen for various periods of time. The changes induced in the stimulated groups are compared with changes in unstimulated cell populations. Radiolabelled amino acids are convenient as they provide a means of quantitating the changes in a simple, reproducible manner. Thus, as used herein, the expression “assay of proliferative activity” means the following assay:
In vitro proliferation is accomplished using freshly recovered splenocytes from BALB/c mice seeded at a density of 5×10⁴ cells/well and incubated for 24, 48 and 72 h with increasing concentrations of total microorganism supernatants or recombinant TcPa45 protein or other mitogen (0.07-200 μg/ml) with 0.5 μg/ml of the HPLC fractions, or with the conventionally used mitogens concanavalin A (10 μg/ml) and lipopolysaccharide (5 μg/ml) in 5% FCS in RPMI-1640 complete medium. T-cell depletion is accomplished by incubating freshly recovered spleen cells for 30 min at 37° C. in the presence of monoclonal antibodies against Thy 1.2 and rabbit complement (Cedarlane, Le Perray en Yvelines, France). Analysis of proliferative activity of total splenocytes (5×10⁴ cells/well) in the presence of 50 μg/ml enzymatically active rTcPA45 or other mitogen is also compared with the proliferation obtained using the same amounts of rTcPA45 protein or other mitogen lacking racemase activity (by heating for 10 min at 80° C. or by long term storage at 4° C.). Inhibition of proliferation is obtained by adding to the splenocyte cultures 50 μg/ml rTcPA45 or other mitogen pre-incubated for 10 min at 37° C. with 1 mM inhibitor, either specific (pyrrole-2-carboxylic acid) or nonspecific (iodoacetamide or iodoacetate). Competitive assays of proliferative activity by 50 μg/ml rTcPA45 or other mitogen are done by adding increasing concentrations of specific substrates i.e., proline racemase substrates (L- or D-proline) for the mitogen rTcPA45 ranging from 3 mM to 50 mM. Controls include the incubation of splenocytes (5×10⁴ cells/well) with substrate alone, i.e., 50 mM L- or D-proline in RPMI medium alone for proline racemase. Cultures are collected after a 16-hour pulse or 1 μCi/well and ³H-thymidine uptake is determined by liquid scintillation counting. All data points are obtained in triplicate and the corresponding standard deviation is calculated.

This assay of proliferative activity is used to determine whether a substance is a mitogen. This assay is also used to determine a sub-mitogenic amount of the mitogen.

As used herein, the term “sub-mitogenic amount” means an amount of the parasite mitogen which is less than an amount of the parasite mitogen that produces an increase in lymphocyte proliferation in the assay of proliferative activity. Thus, the sub-mitogenic amount can be easily determined by carrying out the assay of proliferative activity at several low dosages of the parasite mitogen and noting the dosage at which proliferative activity first increases. The sub-mitogenic amount is an amount below the dosage at which proliferative activity first increases.

The sub-mitogenic amount must also be sufficient to induce protective immunity against the microorganism in a host to which the sub-mitogenic amount of the parasite mitogen is administered. As used herein, the term “protective immunity” refers to an adaptive (specific) immune response characterized by specificity and memory in the host to which the antigens, antibodies, or mitogen is administered. The adaptive immune response once stimulated by an invading microorganism will remember and respond more rapidly to infection so that no disease will occur or any disease that occurs following infection will be less severe as compared to a similar infection without prior immunization according to the invention. Thus, the protective immunity imparted by the method of the invention imparts protection from disease, particularly infectious disease, as evidenced by the absence of clinical indications of disease, or as evidenced by absence of, or reduction in, determinants of pathogenicity, including the absence or reduction in persistence of the infectious microorganism or virus in vivo, and/or the absence of pathogenesis and clinical disease, or diminished severity thereof, as compared to individuals not treated by the method of the invention.

In practicing the method of the invention, the antigens, antibodies, or mitogens are administered to a host using one of the modes of administration commonly employed for administering drugs to humans and other animals. Thus, for example, the antigens, antibodies, or mitogens can be administered to the host by the oral route or parenterally, such as by intravenous or intramuscular injection. Other modes of administration can also be employed, such as intrasplenic, intradermal, and mucosal routes. For purposes of injection, the antigens, antibodies, or mitogens described above can be prepared in the form of solutions, suspensions, or emulsions in vehicles conventionally employed for this purpose.

It will be understood that the antigens, antibodies, or mitogens of the invention can be used in combination with other microorganism or viral antigens, antibodies, or mitogens or other prophylactic or therapeutic substances. For example, mixtures of different parasite antigens, antibodies, or mitogens or mixtures of different bacterial antigens, antibodies, or mitogens can be employed in the method of the invention. Similarly, mixtures of antigens, antibodies, or mitogens can be employed in the same composition. The antigens, antibodies, or mitogens can also be combined with other vaccinating agents for the corresponding disease, such as microbial immunodominant, immunopathological, and immunoprotective epitope-based vaccines or inactivated attenuated, or subunit vaccines. The microorganism and viral antigens, antibodies, or mitogens can even be employed as adjuvants for other immunogenic or vaccinating agents.

The antigens, antibodies, or mitogens of the invention are employed in an amount sufficient to provide an adequate concentration of the drug to prevent or at least inhibit infection of the host in vivo or to prevent or at least inhibit the spread of the microorganism in vivo. The amount of the antigens, antibodies, or mitogens thus depends upon absorption, distribution, and clearance by the host. Of course, the effectiveness of the antigens, antibodies, or mitogens is dose related. The dosage of the antigens, antibodies, or mitogens should be sufficient to produce a minimal detectable effect, but the dosage should be less than the dose that activates a non-specific polyclonal lymphocyte response as measured by the assay of proliferative activity previously described.

The dosage of the antigens, antibodies, or mitogens of the invention administered to the host can be varied over wide limits. The antigens, antibodies, or mitogens can be administered in the minimum quantity, which is therapeutically effective, and the dosage can be increased as desired up the maximum dosage tolerated by the patient. The antigens, antibodies, or mitogens can be administered as a relatively high amount, followed by lower maintenance dose, or the antigens, antibodies, or mitogens can be administered in uniform dosages.

The dosage and the frequency of administration will vary with the antigens, antibodies, or mitogens employed in the method of the invention. In the case of the TcPA45 parasite mitogen, the sub-mitogenic amount administered to a human can vary from about 50 ng per Kg of body weight to about 1 μg per Kg of body weight, preferably about 100 ng per Kg of body weight to about 500 ng per Kg of body weight. Similar dosages can be employed for the other mitogens employed in this invention but optimum amounts can be determined with a minimum of experimentation using conventional dose-response analytical techniques or by scaling up from studies based on animal models of disease.

The term “about” as used herein in describing dosage ranges means an amount that is equivalent to the numerically stated amount as indicated by the induction of protective immunity in the host to which the antigens, antibodies, or mitogens are administered, with the absence or reduction in the host of determinants of pathogenicity, including an absence or reduction in persistence of the infectious microorganism in vivo, and/or the absence of pathogenesis and clinical disease, or diminished severity thereof, as compared to individuals not treated by the method of the invention.

The dose of the antigens, antibodies, or mitogens of the invention is specified in relation to an adult of average size. Thus, it will be understood that the dosage can be adjusted by 20-25% for patients with a lighter or heavier build. Similarly, the dosage for a child can be adjusted using well known dosage calculation formulas.

The antigens, antibodies, or mitogens of the invention can be used in therapy in the form of pills, tablets, lozenges, troches, capsules, suppositories, injectable in ingestable solutions, and the like in the treatment of cytopathic and pathological conditions in humans and susceptible non-human primates and other animals.

Appropriate pharmaceutically acceptable carriers, diluents, and adjuvants can be combined with the antigens, antibodies, or mitogens described herein in order to prepare the pharmaceutical compositions for use in the treatment of pathological conditions in animals. The pharmaceutical compositions of this invention contain the active antigens, antibodies, or mitogens together with a solid or liquid pharmaceutically acceptable nontoxic carrier. Such pharmaceutical carriers can be sterile liquids, such as water an oils, including those of petroleum, animal, vegetable, or synthetic origin. Examples of suitable liquids are peanut oil, soybean oil, mineral oil, sesame oil and the like. Water is a preferred carrier when the pharmaceutical composition is administered intravenously. Physiological solutions can also be employed as liquid carriers, particularly for injectable solutions.

The ability of the vaccines of the invention to induce protection in a host can be enhanced by emulsification with an adjuvant, incorporation in a liposome, coupling to a suitable carrier, or by combinations of these techniques. For example, the vaccines of the invention can be administered with a conventional adjuvant, such as aluminum phosphate and aluminum hydroxide gel. Similarly, the vaccines can be bound to lipid membranes or incorporated in lipid membranes to form liposomes. The use of nonpyrogenic lipids free of nucleic acids and other extraneous matter can be employed for this purpose.

Suitable pharmaceutical excipients include starch, glucose, lactose, sucrose, gelatine, malt, rice, flour, chalk, silica gel, magnesium carbonate, magnesium stearate, sodium stearate, glycerol monstearate, talc, sodium chloride, dried skim milk, glycerol, propylene glycol, water, ethanol, and the like. These compositions can take the form of solutions, suspensions, tablets, pills, capsules, powders, sustained-release formulations and the like. Suitable pharmaceutical carriers are described in “Remington's Pharmaceutical Sciences” by E. W. Martin. The pharmaceutical compositions contain an effective therapeutic amount of the antigens, antibodies, or mitogens of the invention together with a suitable amount of carrier so as to provide the form for proper administration to the host.

The host or patient can be an animal susceptible to infection by the microorganism, and is preferably a mammal. More preferably, the mammal is selected from the group consisting of a human, a dog, a cat, a bovine, a pig, and a horse. In an especially preferred embodiment, the mammal is a human.

Another aspect of the invention includes administering nucleic acids encoding antigens, antibodies, or mitogens of the invention with or without carrier molecules to an individual. Those of skill in the art are cognizant of the concept, application, and effectiveness of nucleic acid vaccines (e.g., DNA vaccines) and nucleic acid vaccine technology as well as protein and polypeptide based technologies. The nucleic acid based technology allows the administration of nucleic acids encoding antigens, antibodies, or mitogens of the invention, naked or encapsulated, directly to tissues and cells without the need for production of encoded proteins prior to administration. The technology is based on the ability of these nucleic acids to be taken up by cells of the recipient organism and expressed to produce an antigen, antibody, or mitogen to which the recipient's immune system responds. Such nucleic acid vaccine technology includes, but is not limited to, delivery of naked DNA and RNA and delivery of expression vectors encoding an antigen, antibody, or mitogen of the invention. Although the technology is termed “vaccine,” it is equally applicable to immunogenic compositions that do not result in a complete protective response. Such partial-protection-inducing compositions and methods are encompassed within the present invention.

Although it is within the present invention to deliver nucleic acids encoding the antigens, antibodies, or mitogens of the invention as naked nucleic acids, the present invention also encompasses delivery of nucleic acids as part of larger or more complex compositions. Included among these delivery systems are viruses, virus-like particles, or bacteria containing the nucleic acids encoding the antigens, antibodies, or mitogens of the invention. Also, complexes of the invention's nucleic acids and carrier molecules with cell permeabilizing compounds, such as liposomes, are included within the scope of the invention. Other compounds, such as molecular vectors (EP 696,191, Samain et al.) and delivery systems for nucleic acid vaccines are known to the skilled artisan and exemplified in, for example, WO 93 06223 and WO 90 11092, U.S. Pat. No. 5,580,859, and U.S. Pat. No. 5,589,466 (Vical patents), which are incorporated by reference herein, and can be made and used without undue or excessive experimentation.

The platform of the invention relates to reagents, systems and devices for performing the process of screening of D-amino acid tests.

This invention further contemplates:
1. Any molecular modification of the gene or a fragment of the gene encoding for a racemase/epimerase that leads to the inhibition of the expression of the protein by the microorganism, especially parasite or bacteria (gene knock out), and further utilization of parasites or bacteria lacking those activities in vivo aiming at immunoprotective responses.
2. Any molecular modification of the gene or a fragment of the gene encoding for a racemase/epimerase that leads to the hyperexpression of the protein by the microorganism, especially parasite or bacteria (gene transgenesis), and further utilization of the microorganism, especially parasite or bacteria to produce high amounts of the protein aiming at producing high amounts of the native protein.
3. Any molecular modification of the gene or a fragment of the gene encoding a racemase/epimerase that leads to an attenuation of microorganism, especially parasite or bacterial infectivity, or interaction with a host cell, and further injection of the parasites or bacteria in vivo aiming at immunoprotective responses.
4. Any molecular modification (for instance directed mutagenesis) of the protein or of its active site that leads to the inhibition of its enzymatic or its mitogenic activity and further injection of mutated microorganisms, especially parasites or bacteria in vivo aiming at immunoprotective responses.
5. Use of any molecular or biochemical modification of the enzymatic activity of the racemase or epimerase (inhibition of the active site) aiming at developing specific immunotherapy.
6. Any molecule or compound that inhibits the enzymatic activity of the protein aiming at developing a drug against microorganism, especially parasite or bacterial infection or specific treatment of microorganismal, especially parasitic or bacterial disease.

This invention will now be described with reference to the following examples.

### EXAMPLE 1

### Cloning and Automated Sequencing

Lambda phage and plasmid DNA were prepared using standard techniques and direct sequencing was accomplished with the Big dye Terminator Kit (Perkin Elmer, Montigny-le Bretonneux, France) according to the manufacturer's instructions. Extension products were run for 7 h in an ABI 377 automated sequencer. Briefly, to obtain the full length of the TcPRAC gene, ³²P-labeled 239 bp PCR product was used as a probe to screen a T. cruzi clone CL-Brener lamba Fix II genomic library (see details in (13)). There were isolated 4 independent positive phages. Restriction analysis and Southern blot hybridization showed two types of genomic fragments, each represented by 2 phages. Complete sequence and flanking regions of representative phages for each pattern was done. Complete characterization of TcPRACA gene, representing the first phage type, was previously described in (13). Full sequence of the putative TcPRACB gene, representing the second phage type was then performed and primers internal to the sequence were used for sequencing, as described before (13).

### EXAMPLE 2

### Chromoblots

Epimastigote forms T. cruzi (clone CL Brener) are maintained by weekly passage in LIT medium. Agarose (0.7%) blocks containing 1×10⁷ cultured parasites were lysed with 0.5 M EDTA/10 mM Tris/1% sarcosyl pH 8.0, digested by proteinase K and washed in 10 mM Tris/1 mM EDTA, pH 8.0. Pulsed field gel electrophoresis (PFGE) was carried out at 18° C. using the Gene Navigator apparatus (Pharmacia, Upsala, Sweden) in 0.5×TBE. Electrophoresis were performed, as described in (14). Gels were then stained with ethidium bromide, photographed, exposed to UV light (265 nm) for 5 min and further blotted under alkaline conditions to a nylon filter (HybondN+, Amersham Life Science Inc., Cleveland, USA). DNA probes, obtained by PCR amplification of TcPRACA gene with Hi-45 (5′ CTC TCC CAT GGG GCA GGA AAA GCT TCT G 3′) [SEQ ID NO:5] and Bg-45 (5′ CTG AGC TCG ACC AGA T(CA)T ACT GC 3′) [SEQ ID NO:6] oligonucleotides (as described in (13)) were labelled with αdATP³² using Megaprime DNA labelling system (Amersham). The chromoblot was hybridized overnight in 2×Denhart's/5×SSPE/1.5% SDS at 55° C. and washed in 2×SSPE/0.1% SDS followed by 1×SSPE at 60° C. Autoradiography was obtained by overnight exposure of the chromoblot using a Phosphorimager cassette (Molecular Dynamics, UK).

### EXAMPLE 3

### Plasmid Construction and Protein Purification

The TcPRACA gene fragment starting at codon 30 was obtained by PCR, using Hi- and Bg45 primers, and cloned in frame with a C-terminal six-histidine tag into the pET28b(+) expression vector (Novagen-Tebu, Le Perray en Yvelines, France). The fragment encoding the TcPRACB consisted of a HindIII digestion of TcPRACB gene fragment obtained by similar PCR and cloned in frame with a C-terminal six-histidine tag into the pET28b(+) expression vector. Respective recombinant proteins TcPRACA and TcPRACB were produced in E. coli BL21 (DE3) (Invitrogen, Cergy Pontoise, France) and purified using Immobilized Metal Affinity Chromatography on nickel columns (Novagen-Tebu, Le Parrayen Yvelines, France) following the manufacturer's instructions.

### EXAMPLE 4

### Size Exclusion Chromatography

rTcPRACA and rTcPRACB proteins were purified as described here above and dialysed against PBS pH 7.4 or 0.2 M NaOAc pH 6.0 elution buffers in dialysis cassettes (Slide-A-lyzer 7K Pierce), overnight at 4° C. The final protein concentration was adjusted to 2 mg/ml and 0.5 ml of the solution were loaded onto Pharmacia Superdex 75 column (HR10×30), previously calibrated with a medium range protein calibration kit (Pharmacia). Size exclusion chromatography (SEC) was carried out using an FPLC system (AKTA Purifier, Pharmacia). Elution was performed at a constant flow rate of 0.5 ml/min, protein fractions of 0.5 ml were collected and the absorbance was monitored at 280 nm. Each fraction was assayed in racemization assays as described here below. Fractions B1 and B5, were reloaded in the Superdex 75 column and submitted to a further SEC to verify the purity of the fractions.

### EXAMPLE 5

### Racemization Assays

The percent of racemization with different concentrations of L-proline, D-proline, L-hydroxy (OH)-proline, D-hydroxy (OH)-proline was calculated, as described in (13), by incubating a 500 μl mixture of 0.25 μM of dimeric protein and 40 mM substrate in 0.2 M sodium acetate pH 6.0 for 30 min or 1 h at 37° C. The reaction was stopped by incubating for 10 min at 80° C. and freezing. Water (1 ml) was then added, and the optical rotation was measured in a polarimeter 241 MC (Perkin Elmer, Montigny le Bretonneux, France) at a wavelength of 365 nm, in a cell with a path length of 10 cm, at a precision of 0.001 degree. The percent of racemization of 40 mM L-proline as a function of pH was determined using 0.2 M sodium acetate, potassium phosphate and Tris-HCl buffers; reactions were incubated 30 min at 37° C., as described above. All reagents were purchased from Sigma.

### EXAMPLE 6

### Kinetic Assays

Concentrations of L- and D-proline were determined polarimetrically from the optical rotation of the solution at 365 nm in a cell of 10 cm path length, thermostated at 37° C. Preliminary assays were done with 40 mM of L-proline in 0.2 M sodium acetate pH 6 in a final volume of 1.5 ml. Optical rotation was measured every 5 sec during 10 min and every 5 min to 1 hour. After determination of the linear part of the curve, velocity in 5-160 mM substrate was measured every 30 sec during 10 min to determine K_{M} and Vₘₐₓ. Calculations were done using the Kaleïdagraph® program. Inhibition assays were done by incubating 0.125 μM dimeric protein, 6,7 μM-6 mM pyrrole-2-carboxylic acid (PAC), 20 to 160 mM L-proline, as described above. Graphic representation and linear curve regression allowed the determination of Kᵢ as [PAC]/[(slope with PAC/slope without PAC)−1]. All reagents were purchased from Sigma.

### EXAMPLE 7

### Site-Directed Mutagenesis of ^{C330S}TcPRACA

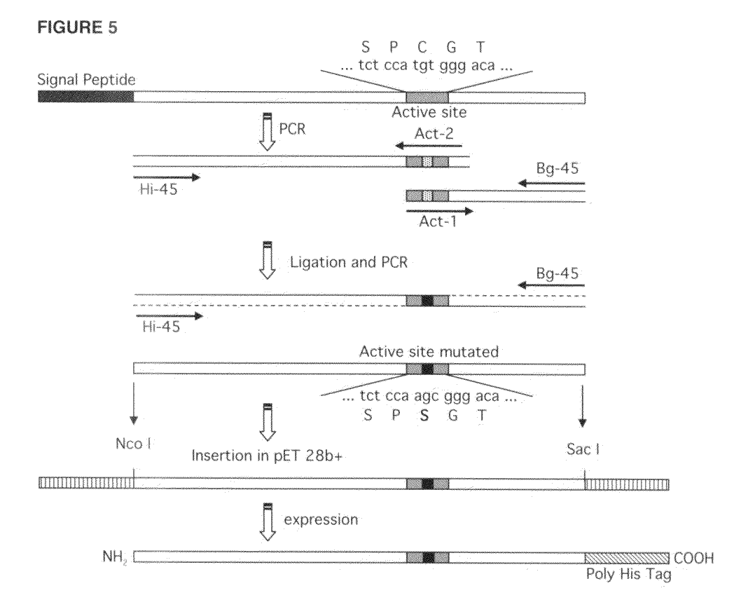

Site-directed mutagenesis was performed by PCR, adapting the method of Higuchi et al. (52). Briefly, mutation of Cys³³⁰ of the proline racemase active site was produced by two successive polymerase chain reactions based on site-directed mutagenesis using two overlapping mutagenic primers: (act-1) 5′ GCG GAT CGC TCT CCA AGC GGG ACA GGC ACC 3′ [SEQ ID NO:7] and (act-2) 5′ GGT GCC TGT CCC GCT TGG AGA GCG ATC CGC 3′, [SEQ ID NO:8] designed to introduce a single codon mutation in the active site by replacement of the cysteine (TGT) at the position 330 by a serin (AGC). A first step standard PCR amplification was performed using the TcPRACA DNA as template and a mixture of act-1 primer and the reverse C-terminus primer (Bg-45) 5′ CTG AGC TCG ACC AGA T(CA)T ACT GC 3′ (codon 423), or a mixture of act-2 primer and the forward N-terminus primer (Hi-45) 5′ CTC TCC CAT GGG GCA GGA AAA GCT TCT G 3′ (codon-53) (see FIG. 5). Resulting amplified fragments of, respectively, 316 bp and 918 bp were purified by Qiagen® PCR extraction kit (Qiagen, Courtaboeuf, France), as prescribed, and further ligated by T4 ligase to generate a template consisting of the full length of a potentially mutated TcPRACA* coding sequence used for the second step PCR. Amplification of this template was performed using forward Hi-45 and reverse Bg-45 primers and the resulting TcPRACA* fragment encoding for the mature proline racemase was purified and cloned in pCR®2.1-TOPO® vector (Invitrogen). TOP10 competent E. coli were transformed with the pCR®2.1-TOP®-TcPRACA* construct and plasmid DNA isolated from individual clones prepared for DNA sequencing. Positive mutants were then sub-cloned in frame with a C-terminal six-histidine tag into the Nco I/Sac I sites of the pET 28b(+) expression vector (Novagen-Tebu, Le Parrayen Yvelines, France). Sub-clones of pET28b(+)-TcPRACA* produced in E. coli (DH5α) were sequenced again to confirm the presence of the mutation. Soluble recombinant ^{C330S}TcPRACA protein was produced in E. coli BL21 (DE3) (Invitrogen) and purified using a nickel column (Novagen-Tebu), according the manufacturer's instructions.

### EXAMPLE 8

### Mutagenesis

To verify the implication of the residue Cys160 in the reaction mechanism of the proline racemase, a site specific mutagenesis was performed to replace the residue Cys160 by a Serine, similarly to mutation described for Cys330 residue (see Example 7). Briefly, the site specific mutagenesis was performed by PCR using the following primers:

Ser160-Forward:
^{5′}GGCTATTTAAATATGTCTGGACATAACTCAATTGCAGCG^{3′}
Ser160-Reverse:
^{5′}CGCTGCAATTGAGTTATGTCCAGACATATTTAAATAGC^{3′}

The presence of the mutation Cysteine-Serine was verified by sequencing of the respective plasmids containing the PCR products, as shown here below. The plasmid pET-C160S was used to transform E. coli BL21 (DE3) and to produce the corresponding recombinant mutated protein.

| | | | |
|---|---|---|---|
| | 139 | M D T C G Y L N M C G H N G I A A | 145 |
| pET-TcPRAC | 499 | ATCGATACCGCTGGCTATTTAAATATGTGTGGACATAACTCAATTGCAGCG | 550 |
| Ser160-F/R | | GGCTATTTAAATATGTCTGGACATAACTCAATTGCAGCG | 550 |
| pET-C160S | 499 | ATGGATACCGGTGGCTATTTAAATATGTCTGGACATAACTCAATTGCAGCG | 550 |
| pET-C330S | 499 | ATGGATACCGGTGGCTATTTAAATATGTGTGGACATAACTCAATTGCAGCG | 550 |
| | 139 | M D T C G Y L N M S G H N G I A A | 145 |
| | 318 | V I F G N R Q A D R S P C G T C T | 334 |
| pET-TcPRAC | 999 | GTGATATTTGGCAATCGCCAGGCGGATCGCTCTCCATGTGGGACAGGCACC | 1050 |
| Ser330-F/R | | GCGGATCGCTCTCCAAGCGGGACAGGCACC | 1050 |
| pET-C160S | 999 | GTGATATTTGGCAATCGCCAGGCGGATCGCTCTCCATGTGGGACAGGCACC | 1050 |
| pET-C330S | 999 | GTGATATTTGGCAATCGCCAGGCGGATCGCTCTCCAAGCGGGACAGGCACC | 1050 |
| | 318 | V I F G N R Q A D R S P S G T C T | 334 |

Underlined are the primer sequences used for the site specific mutageneses. The mutations Cys→Ser are represented in bold and underlined for both Cys160 and Cys330 residues.

### EXAMPLE 9

### Expression of a Functional Intracellular Isoform of Proline Racemase

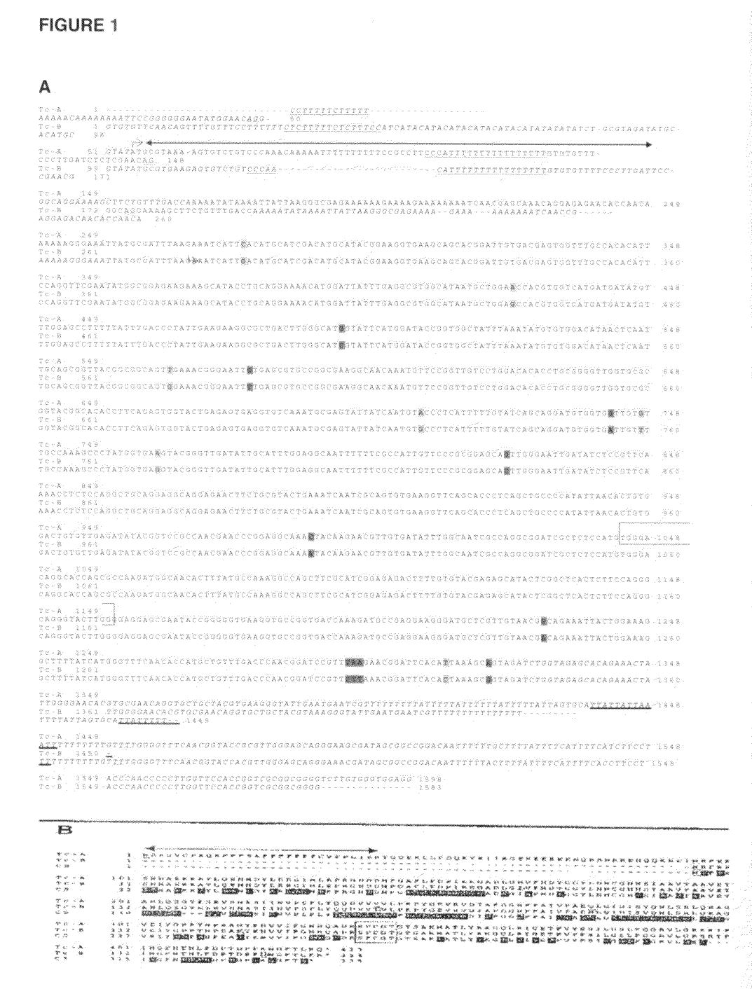

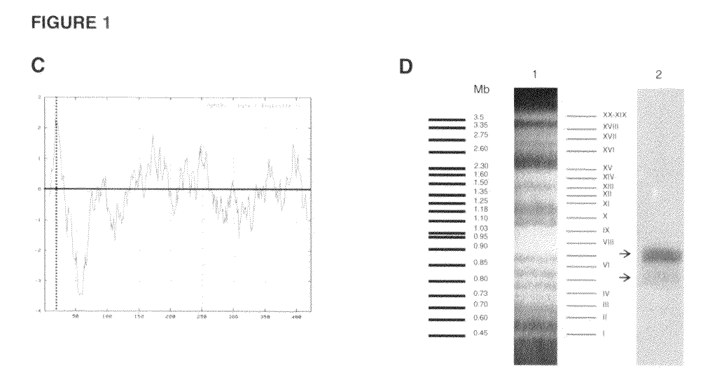

Previously characterized was a TcPRAC gene from T. cruzi, and it was demonstrated in vivo and in vitro that it encodes a proline racemase enzyme (13). Analysis of the genomic organization and transcription of the TcPRAC gene indicated the presence of two paralogue gene copies per haploid genome, named TcPRACA¹ and TcPRACB². It was shown that TcPRACA encodes a functional co-factor independent proline racemase, closely resembling the C. sticklandii proline racemase (CsPR) (11). Now sequenced was the full length of TcPRACB and, as can be observed in FIG. 1A, TcPRACA and TcPRACB genes both possess the characteristic trypanosome polypyrimidine-rich motifs in the intergenic region that are crucial trans-splicing signals when located upstream of an (AG)-dinucleotide used as acceptor site. As in other T. cruzi genes, UUA triplets are found at the end of the 3′ untranslated region preceding the polyadenylation site. Comparison between the two sequences revealed 14 point mutations (resulting in 96% identity) giving rise to 7 amino acid differences. When expressed, the TcPRACB is predicted to produce a shorter protein (39 kDa) whose translation would start at the ATG codon at position 274 located downstream of the (AG)-spliced leader acceptor site (at position 175). In comparison, TcPRACA has an open reading frame that encodes a peptide with an apparent molecular mass of 45 kDa. The schematic protein sequence alignment of the two proteins TcPRACA and TcPRACB depicted in FIG. 1B reveals that TcPRACB proline racemase lacks the amino acid sequence corresponding to the signal peptide observed in the TcPRACA protein (hatched box in the figure; see predicted cleavage site in FIG. 1C). Therefore the TcPRACB would produce a 39 kDa, intracellular and non-secreted isoform of the protein. As with CsPR (11) and TcPRACA (13 and FIG. 1B), the active site of proline racemase is conserved in TcPRACB sequence. Furthermore, while differing by only 7 amino acids, both the TcPRACA and TcPRACB sequences display around 50% homology to the CsPR (13). In accordance with other protein-coding genes in T. cruzi, TcPRAC genes are located on two different chromosomal bands of which one contains three or more chromosomes of similar size, see FIG. 1D. Thus, hybridization of blots containing T. cruzi CL Brener chromosomal bands separated by pulsed field gel electrophoresis revealed that sequences recognized by an homologous probe to both TcPRACA and TcPRACB are mapped in neighboring migrating bands of approximately 0.9 Mb and 0.8 Mb, corresponding respectively to regions VII and V, according to Cano et al. (51).

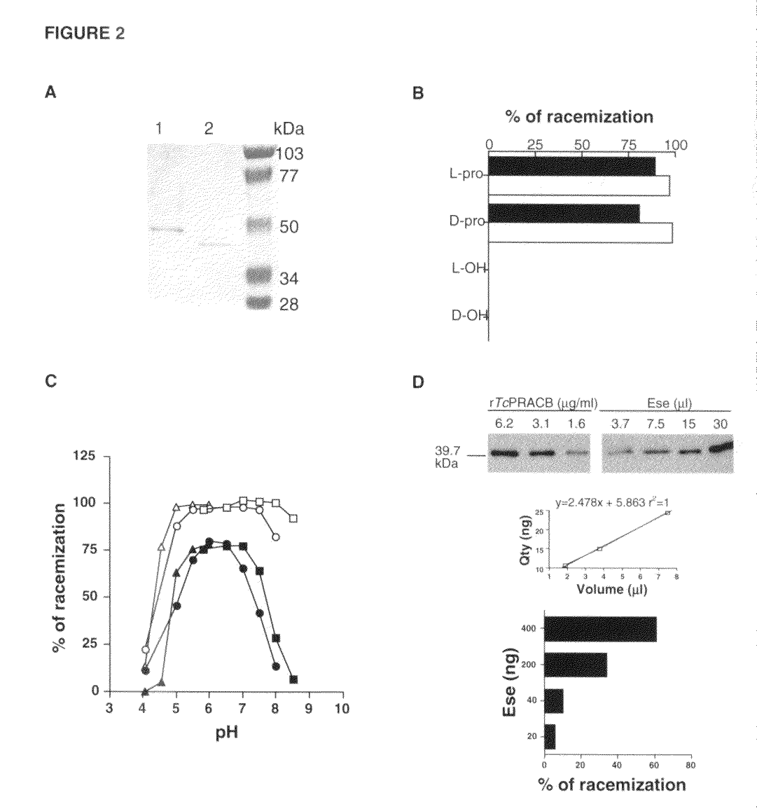

In order to verify if the TcPRACB gene could encode a functional proline racemase, both T. cruzi paralogues were expressed in E. coli to produce C-terminal His₆-tagged recombinant proteins. After purification by affinity chromatography on nickel-nitrilotriacetic acid agarose column, recombinant proteins were separated by SDS gel electrophoresis revealing single bands with the expected sizes of 45.8 and 40.1 kDa, respectively, for the rTcPRACA and rTcPRACB proteins (FIG. 2A). To determine whether rTcPRACB displays proline racemase enzymatic activity, biochemical assays were employed to measure the shift in optical rotation of L- and D-proline substrates, as described (13). As can be seen in FIG. 2B, rTcPRACB racemizes both L- and D-proline but not L-hydroxy-proline, like rTcPRACA. In a similar manner, rTcPRACB is a co-factor independent proline racemase as described for CsPR (11) and rTcPRACA (13) proline racemases. The rate of conversion of L- into D-proline was measured at various pH values using both recombinant enzymes. As illustrated in FIG. 2C, rTcPRACA activity clearly shows a pH dependency with an optimal activity from pH 5.5 to 7.0. In contrast, the optimum activity of rTcPRACB can be observed in a large pH spectrum varying from pH 4.5 to 8.5. These results revealed that translation of the open reading frame of both TcPRAC genes copies result in functional proline racemase isoforms. As previously described, Western blot analysis of non-infective epimastigote parasite extracts using antibodies raised against the 45 kDa secreted proline racemase had previously revealed a 39 kDa protein mostly in the soluble cellular fraction, only weakly in the cellular insoluble fraction and absent from culture medium (13). To demonstrate that the intracellular 39 kDa isoform of the protein was equally functional in vivo, soluble cellular extracts were obtained from 5×10⁸ epimastigotes, non-infective parasites and the levels of 39 kDa soluble protein quantified by Western blot comparatively to known amounts of rTcPRACB enzyme. As can be observed in FIG. 2D, the intracellular isoform of the protein is indeed functional in vivo, since proline racemase enzymatic activity was displayed and levels of racemization were dependent on protein concentration. This discovery is useful for specific inhibitors reaching the intracellular compartment.

### EXAMPLE 10

### Functional Analysis and Kinetic Properties of Recombinant T. cruzi Proline Racemases

Since the TcPRAC gene copies encode for secreted and non-secreted isoforms of proline racemase with distinct pH requirements for activity, our investigation was made to determine whether other biochemical properties differ between rTcPRACA and rTcPRACB proteins. Such differences might reflect the cellular localization of the protein during parasite differentiation and survival in the host. Both rTcPRACA and rTcPRACB enzyme activities are maximal at 37° C. and can be abolished by heating for 5 min at 80° C. However, the stability of the two recombinant enzymes differs considerably, when analyzed under different storage conditions. Thus, as shown in Table II, purified rTcPRACB is highly stable, since its activity is maintained for at least 10 days at room temperature in 0.5 M imidazol buffer pH 8.0, as compared to rTcPRACA that loses 84% of its activity under such conditions. In contrast, most of the enzymatic activity of rTcPRACA is maintained at 4° C. (65%), compared to that of rTcPRACB (34%). Both enzymes can be preserved in 50% glycerol at −20° C., or diluted in sodium acetate buffer at pH 6.0, but under these storage conditions rTcPRACA activity is impaired. However, best preservation of both recombinant proline racemases was undoubtedly obtained when proteins were kept at −20° C. as ammonium sulfate precipitates. Preservation is important for a kit.

TABLE II
Stability of recombinant TcPRACA and TcPRACB proline
racemases under different storage conditions
% of preservation of proline racemase activity

| | Column | | | | NaOAc | (NH₄)₂SO₄ | |
|---|---|---|---|---|---|---|---|
| Protein | CTRL | RT | +4° C. | Gly/−20° C. | pH 6 4° C. | 4° C. | −20° C. |
| rTcPRACA | 100.0 | 16.0 | 66.5 | 62.9 | 31.0 | 53.9 | 100.0 |
| rTcPRACB | 100.0 | 100.0 | 34.0 | 93.6 | 77.6 | 98.4 | 100.0 |

After purification on nickel-nitrilotriacetic acid agarose column, recombinant proteins were kept for 10 days in nickel column buffer (20 mM Tris/500 mM NaCl/500 mM imidazol, pH 8.0) at room temperature (RT) or at +4° C., or either diluted in 50% glycerol and maintained at −20° C. (Gly/−20° C.) or in optimum pH buffer (NaOAc, pH 6.0) at 4° C. Recombinant enzymes were precipitated in (NH₄)₂SO₄ and kept in solution at 4° C. or pellet dried at −20° C. Racemase assays were performed for 30 min at 37° C. Percent of preservation was determined polarimetrically using 0.25 μM of either purified rTcPRACA or rTcPRACB enzymes and 40 mM of L-proline, as compared to results obtained with freshly purified proteins (CTRL). These results are representative of at least two independent experiments.

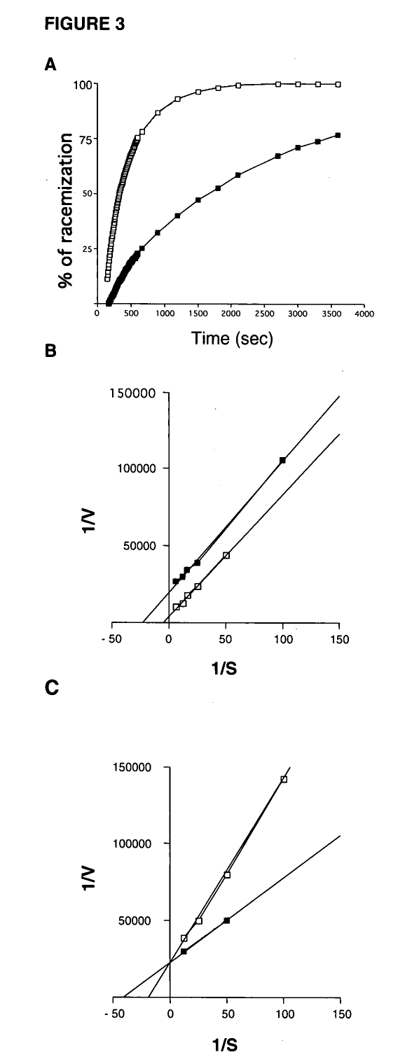

Both recombinant enzymes exhibited Michaelis-Menten kinetics (FIG. 3A) and rTcPRACB had a higher activity than rTcPRACA. Indeed, as can be observed in FIG. 3B, analysis of L to D conversion of serial dilutions of L-proline catalyzed by a constant amount of each enzyme showed that rTcPRACB enzyme (K_{M} of 75 mM and Vₘₐₓ of 2×10⁻⁴ mol.sec⁻¹) has a higher velocity as compared to rTcPRACA (K_{M} of 29 mM and Vₘₐₓ of 5.3×10⁻⁵ mol.sec⁻¹). In order to determine the Kᵢ values for pyrrole-2-carboxylic acid (PAC), the specific and competitive inhibitor of CsPR (16), assays were performed with both recombinant proteins. These assays revealed that PAC is comparably effective as an inhibitor of rTcPRACA (FIG. 3C) and rTcPRACB, and Kᵢ values obtained were, respectively, 5.7 R_{M} and 3.6 μM. The difference in Kᵢ values reflects almost perfectly the difference in K_{M} values reported for both enzymes, which are similar to that of the native protein. These Kᵢ values indicate that the affinity of PAC inhibitor is higher for rTcPRACA and rTcPRACB than for CsPR (Kᵢ of 18 μM). The Kₘ and Kᵢ values are important for an inhibitor.

### EXAMPLE 11

### Requirement of a Dimeric Structure for Proline Racemase Activity

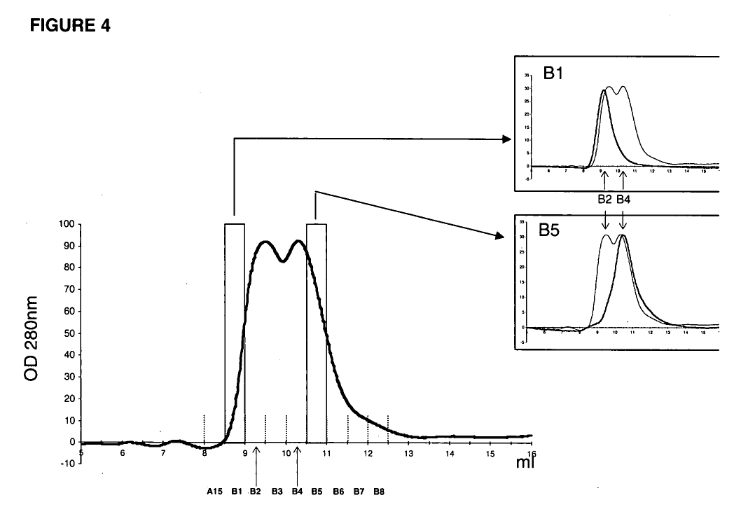

When rTcPRACA was submitted to size exclusion chromatography on a Superdex 75 column at pH 6.0, two peaks of protein were eluted, respectively, around 80 kDa (B2 fraction) and 43 kDa (B4 fraction), presumably corresponding to dimeric and monomeric forms of the enzyme (FIG. 4). Western blot analysis of whole T. cruzi epimastigote extracts using non-denaturing PAGE had previously indicated a molecular mass of 80 kDa for the native protein while a 45 kDa band was obtained by SDS-PAGE (13). In order to eliminate cross-contamination, B1 and B5 fractions, eluted, respectively, at the start and at the end of the predicted dimer (B2) or monomer (B4) peaks, were reloaded on the column and the profiles obtained (see FIG. 4 inserts) confirmed the purity of the fractions. Enzyme activity resides in the 80 kDa peak, but not in the 43 kDa peak (Table III). These results corroborated that two subunits of the protein are necessary for racemase activity. At neutral pH (7.4 or above), the rTcPRACA gives rise to high molecular weight aggregates which are not observed with rTcPRACB, consistently with its broader optimal pH spectrum. The enzyme should be in optimal pH conditions for a kit buffer, for example.

TABLE III
Racemase activity of recombinant TcPRACA fractions
after size exclusion chromatography
Fractions

| | A15 | B1 | B2 | B3 | B4 | B5 | B6 | B7 |
|---|---|---|---|---|---|---|---|---|
| % racemization | 1.3 | 35.5 | 62.9 | 42.8 | 0.7 | 0 | 0 | 0 |

After elution from Superdex 75 column, 20 μl of each peak (A15 to B7, see FIG. 4) corresponding to 1 μg of protein were incubated 1 h at 37° C. with 40 mM of L-proline in 0.2 M NaOAc, pH 6.0. Optical rotation was measured and % of racemization was determined as described in Example 5.

### EXAMPLE 12

### Abrogation of Proline Racemase Activity by Mutation of Cys³³⁰ and Alternately Cys¹⁶⁰ of the Catalytic Site

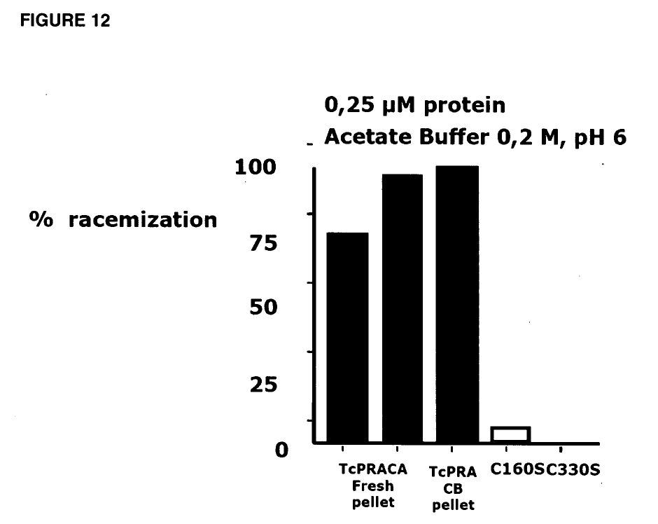

C. sticklandii proline racemase is described as a homodimeric enzyme with subunits of 38 kDa and a single proline binding site for every two subunits, where two cysteines at position 256 might play a crucial role in catalysis by the transfer of protons from and to the bound substrate (12). It has previously been shown that mitogenic properties of the T. cruzi proline racemase are dependent on the integrity of the enzyme active site, as inhibition of B-cell proliferation is obtained by substrate competition and specific use of analogues (PAC) resembling the structure assumed by the substrate proline in its transition state (16). To verify the potential role of the cysteine residues at the active site of the T. cruzi proline racemase, Cys³³⁰ and alternately Cys¹⁶⁰ were replaced by a serine residue through site specific mutation of TcPRACA. The choice of serine as the substituting amino acid was made to avoid further major disturbances on three dimensional structure of the protein (see strategy in FIG. 5 above). After confirmation of the single codon mutation through sequencing of the construct, the C^{330S} or C^{160S} rTcPRACA mutant proline racemase was expressed in E. coli and purified in the same manner as wild type rTcPRACA. Then used were C^{330S} or C^{160S} rTcPRACA in racemization assays to verify the effects of the mutation on the enzymatic activity of the protein. As can be observed in Table IV (and in FIG. 12) a total loss of proline racemase activity is observed as compared to the wild type enzyme, establishing that proton transfer during proline racemization is specifically dependent on the presence of the cysteine residue in the active site.

TABLE IV
Loss of racemase enzymatic activity in the site direct ^{C330S}rTcPRACA

| | rTcPRACA | | | | ^{C330S}rTcPRACA | | | |
|---|---|---|---|---|---|---|---|---|
| | Time (min) | | | | | | | |
| Data set | 0 | 10 | 30 | 60 | 0 | 10 | 30 | 60 |
| Optical rotation | −0.385 | −0.300 | −0.162 | −0.088 | −0.385 | −0.382 | −0.391 | −0.387 |
| % racemization | 0 | 22 | 58 | 77 | 0 | 0 | 0 | 0 |

After purification, 5 μg of rTcPRACA or C330Sr TcPRACA were incubated at 37° C. with 40 mM of L-proline in NaOAc buffer, pH 6.0. Optical rotation was measured at different times and % of racemization was determined as described in Example 5.

### EXAMPLE 13

### Proline Racemase Protein Signatures and Putative Proline Racemases in Sequence Databases

The conservation of critical residues between parasite and bacterial proline racemases prompted a search for similarities between TcPRAC and other protein sequences in SWISS-PROT and TrEMBL databases. Twenty one protein sequences yielded significant homologies, from 11 organisms, such as several proteobacteria of the alpha subdivision (Agrobacterium, Brucella, Rhizobium) and gamma subdivision (Xanthomonas and Pseudomonas), as well as of the fermicutes (Streptomyces and Clostridium). Within the eukaryota, besides in T. cruzi, homologous genes were detected in the human and mouse genomes, where predicted proteins show overall similarities with proline racemase. Except for Clostridium sticklandii and Xantomonas campestri, each other organism encodes 2 paralogues, and Agrobacterium tumefaciens contains 3 genes.

The multiple alignment also allowed for the definition of three signatures of proline racemase, which are described here in PROSITE format. As can be seen in Table V, when using a minimal motif of proline racemase protein (M I), [IVL][GD]XHXXG[ENM]XX[RD]X[VI]XXG, located immediately after the start codon at position 79, the inventors obtained 9 hits. A second motif (M II), consisting of [NSM][VA][EP][AS][FY]X(13,14)[GK]X[IVL]XXD[IV][AS][YWF]GGX[FWY], starting at position 218, gave 14 hits; however, the first or the second half of this motif is not sufficiently stringent to be restrictive for putative proline racemases, but gives hits for different protein families. A third motif (M III), from positions 326 to 339, namely DRSPXGX[GA]XXAXXA, was considered as a minimal pattern for identifying PRAC enzymes. Note that in position 330, the cysteine of the active site was replaced by an X. As shown in Table V, this minimal pattern yields all 21 hits. Curiously, both genes in human as well as in mouse encode threonine instead of cysteine at the X position in motif III, while in Brucella, Rhizobium and Agrobacterium species each encode one protein with C and one with T in this position. One cannot hypothesize the implications of this substitution for the functionality of these putative proteins. If the residue at position 330 is maintained as a cysteine in motif III, a reduced number of 12 hits from 9 organisms is thus obtained, which can probably be considered as true proline racemases.

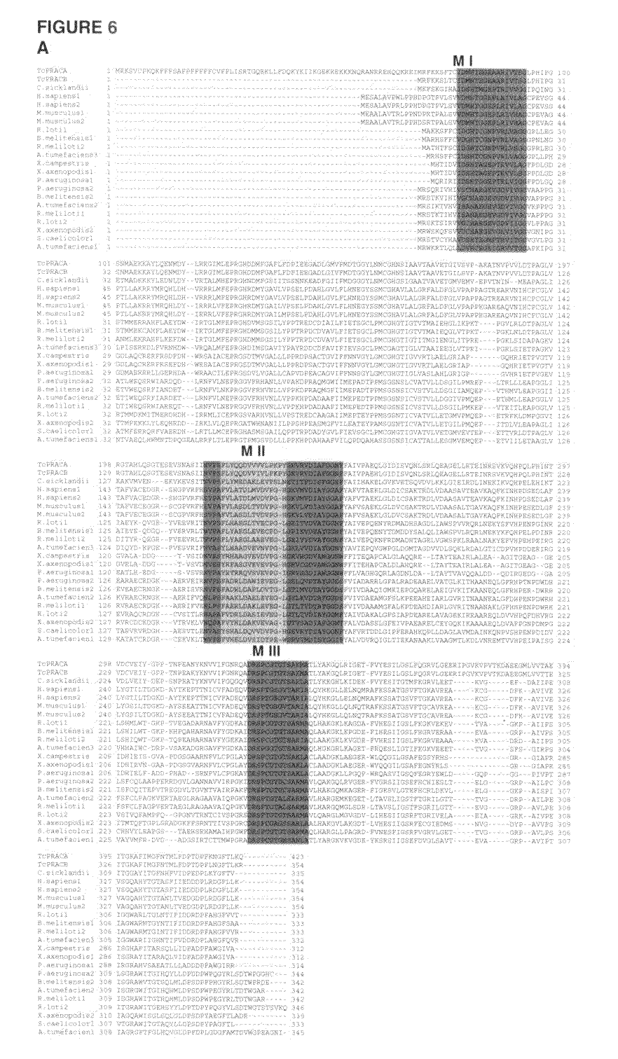

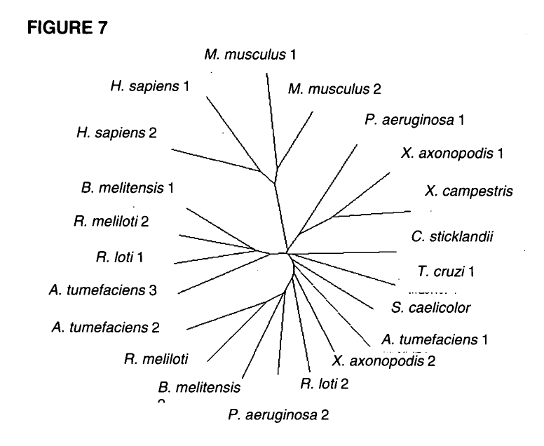

The alignment of the 21 protein sequences and derived cladogram are shown in FIG. 6 and FIG. 7, respectively, the three boxes depicted correspond to motifs I, II and III described here above. Blast searches against unfinished genomes yielded, at present, an additional 13 predicted protein sequences from 8 organisms, with high similarity to proline racemases, all containing motif III. Organisms are Clostridium difficile, C. botulinum, Bacillus anthracis, Brucella suis, Pseudomonas putida, Rhodobacter sphaeroides, Burkholderia pseudomallei, B. mallei, and the fungus Aspergillus fumigatus. These results indicate that proline racemases might be quite widespread.

TABLE V
SWISS-PROT and TrEMBL databases screening
using PROSITE motifs

| | | | Motif | | | |
|---|---|---|---|---|---|---|
| | | | | M | M | M |
| Organism | Seq | Access. nb | M I | II | III | III* |
| Agrobacterium tumefaciens | 1 | Q8UIA0 | + | + | + | + |
| Agrobacterium tumefaciens | 2 | Q8U6X2 | − | − | + | − |
| Agrobacterium tumefaciens | 3 | Q8U8Y5 | − | − | + | − |
| Brucella melitensis | 1 | Q8YJ29 | − | + | + | + |
| Brucella melitensis | 2 | Q8YFD6 | + | − | + | − |
| Clostridium stickilandii | | Q9L4Q3 | − | + | + | + |
| Homo sapiens | 1 | Q96EM0 | + | + | + | − |
| Homo sapiens | 2 | Q96LJ5 | + | + | + | − |
| Mus musculus | 1 | Q9CXA2 | + | + | + | − |
| Mus musculus | 2 | Q99KB5 | + | + | + | − |
| Pseudomonas aeruginosa | 1 | Q9I476 | − | + | + | + |
| Pseudomonas aeruginosa | 2 | Q9I489 | − | − | + | + |
| Rhizobium loti | 1 | Q98F20 | − | + | + | + |
| Rhizobium loti | 2 | Q988B5 | + | + | + | − |
| Rhizobium meliloti | 1 | Q92WR9 | − | − | + | − |
| Rhizobium meliloti | 2 | Q92WS1 | − | + | + | + |
| Streptomyces coelicolor | | Q93RX9 | + | − | + | + |
| Trypanosoma cruzi | 1 | QYNCP4 | + | + | + | + |
| Trypanosoma cruzi | 2 | [symbol] | + | + | + | + |
| Xanthomonas axonopodis | 1 | Q8PJI1 | − | + | + | + |
| Xanthomonas axonopodis | 2 | Q8PKE4 | − | − | + | + |
| Xanthomonas campestris | | Q8P833 | − | + | + | + |
| Bacillus anthracis (Ames) | 1 | Q81UH1 | + | − | + | + |
| Bacillus anthracis (Ames) | 2 | Q81PH1 | − | − | + | + |
| Bacillus cereus | 1 | Q81HB1 | + | − | + | + |
| Bacillus cereus | 2 | Q81CD7 | − | − | + | + |
| Brucella suis | 1 | Q8FYSO | + | + | + | + |
| Brucella suis | 2 | Q8G213 | + | − | + | − |
| Chromobacterium violaceum | | Q7NU77 | + | + | + | + |
| Photorhabdus luminescens | | Q7N4S6 | + | + | + | + |
| Pseudomonas putida | | Q88NF3 | + | + | + | + |
| Rhodopirella baltica | | Q7UWF3 | − | − | + | + |
| Streptomyces avermitilis | | Q82MDO | + | − | + | + |
| Vibrio parahaemolyticus | | Q87Q20 | + | + | + | + |

Table V Legend:
SWISS-PROT and TrEMBL databases were screened using motifs I to III (M I, M II and M III). M I corresponds to [IVL][GD]XHXXG[ENM]XX[RD]X[VI]XXG, M II to of [NSM][VA] [EP][AS][FY]X(13, 14)[GK]X[IVL]XXD[IV][AS][YWF]GGX[FWY] M III to DRSPXGXGXXAXXA and M III* to DRSPCGXGXXAXXA. Access. nb, SWISS-PROT accession number of the sequence;
seq, sequence number according to FIG. 6;
+ and −, presence or absence respectively of hit using the corresponding motif.

Finally, Table VI summarizes the genes in which the proline racemase signature has been identified and the sequences including both crucial residues Cys³³⁰ and Cys¹⁶⁰ of the catalytic site are present.

TABLE VI
Results of screening using nucleotide or peptide sequence of TcPRACA

| | | | Motifs | | | | | common |
|---|---|---|---|---|---|---|---|---|
| | | | | | | M III* | MCGH | sequence |
| Organism | Accession number | Database | M I | M II | M III | Cys³³⁰ | Cys¹⁶⁰ | EPRGH |
| Aspergillus fumigatus | Af0787f05.p1c | TIGR | + | − | + | − | + | + |
| [symbol] | TIGR 5085 | TIGR | + | + | + | + | ? | + |
| Bacillus anthracis str. Ames | AE017027 | EMBL | + | + | + | + | + | + |
| Bacillus anthracis str. Ames | AE017033 | EMBL | + | + | + | + | + | + |
| (minus strand) | | | | | | | | |
| [symbol] | TIGR 1392 | TIGR | + | + | + | + | + | + |
| Bacillus cereus ATCC14579 | AE017007 | EMBL | + | + | + | + | + | + |
| (minus strand) | | | | | | | | |
| Brucella suis 1330 (minus | AE014469 | EMBL | + | + | + | + | + | + |
| strand) | | | | | | | | |
| [symbol] | TIGR 29461 | TIGR | + | + | + | + | + | + |
| Burkholderia mallei | contig: 33162:b_mallei | TIGR | + | + | + | + | + | EPRGSD |
| [symbol] | TIGR 13373 | TIGR | + | ? | + | + | + | EPRGSD |
| [symbol] | SANGER 28450 | Sanger | + | ? | + | + | + | EPRGSD |
| [symbol] | Cbot12g05.q1c | Sanger | ? | + | + | + | + | + |
| [symbol] | SANGER 36826 | Sanger | + | + | + | + | + | + |
| Clostridium difficile | Clostridium difficile | Sanger | ? | + | + | + | + | + |
| | 630 | | | | | | | |
| [symbol] | SANGER 1496 | Sanger | + | + | + | + | + | + |
| Clostridium sticklandii | CST130879 | EMBL | + | + | + | + | + | + |
| [symbol] | LM16BINcontig2054 | Sanger | ? | + | + | + | + | EPRGND |
| [symbol] | LM16W5b02.q1c | Sanger | ? | + | ? | ? | + | EPRGND |
| Pseudomonas putida KT2440 | AE016778 | EMBL | + | + | + | + | + | EPRGND |
| [symbol] | TIGRpputida | TIGR | + | ? | + | + | + | EPRGND |
| KT2440 | 13538 | | | | | | | |
| [symbol] | UTHSC 1063 | UTHSC | + | ? | − | − | + | + |
| [symbol] | TbKIX28b06.qlc | Sanger | ? | + | ? | ? | + | + |
| [symbol] | TbKIX28b06.plc | Sanger | ? | + | ? | ? | + | + |
| [symbol] | Tviv655d02 | Sanger | ? | + | + | + | ? | ? |
| [symbol] | Tviv380d6 | Sanger | + | ? | ? | ? | + | + |
| [symbol] | congo208e06 | Sanger | ? | + | + | + | ? | ? |
| [symbol] | AP005077 | EMBL | + | + | + | + | + | + |

Table VI Legend:
Databases were screened using nucleotide or peptide sequences of TcPRACA. Motifs I to III (M I, M II and M III) were searched. M I corresponds to [IVL][GD]XHXXG[ENM]XX[RD]X[VI]XXG, M II to of [NSM][VA] [EP][AS][FY]X(13, 14)[GK]X[IVL]XXD[IV][AS][YWF]GGX[FWY] M III to DRSPXGXGXXAXXA and M III* to DRSPCGXGXXAXXA. Access. nbs, TIGR, EMBL or SANGER accession numbers of the sequence;
+ and −, presence or absence respectively of the corresponding motif.
Others, extremely conserved regions outside the motifs, including NMCGH which contains one of the active site cysteine.
Sequences presented in annexed pages where the conserved regions of 2 Cysteine residues of the active site are squared, are presented in the table in bold with corresponding Accession numbers.

A variety of free D-amino acids can be found in different mammalian tissues in naturally occurring conditions. Some examples include the presence of D-serine in mammalian brain, peripheral and physiological fluids, or else D-asp that can also be detected in endocrine glands, testis, adrenals and pituitary gland. D-pro and D-leu levels are also very high in some brain regions, pineal and pituitary glands. Some reports attribute to D-amino acids a crucial role as neuromodulators (receptor-mediated neurotransmission), as is the case of D-ser, or as regulators of hormonal secretion, oncogenicity and differentiation (i.e. D-asp). It is believed that the most probable origin of naturally occurring D-amino acids in mammalian tissues and fluids is the synthesis by direct racemization of free L-enantiomers present in situ. However, apart from the cloning of serine racemase genes from rat brain and human no other amino acid racemases were identified until now in man. Some others report that D-amino acids present in mammalian tissues are derived from nutrition and bacteria.

The increasing number of reports associating the presence of D-amino acids and pathological processes indicate that the alteration of their level in biological samples would be of some diagnostic value as, for instance, the identification of changes in free levels of D-asp and D-Ala in brain regions of individuals presenting Alzheimer. The amounts of D-asp seems to decrease in brain regions bearing neuropathological changes and is paralleled by an increase of D-ala. Overall, total amounts of D-amino acids increase in the brain of individuals presenting memory deficits in Alzheimer, as compared to normal brains, offering new insights towards the development of new simple methods of D-amino acid detection. In the same line, D-ser concentrations in the brain are altered in Parkinson disease and schizophrenia but other findings clearly associate significant higher concentrations of D-amino acids in plasma of patients with renal diseases or else in plasma of elderly people.

Previous results determined that the polyclonal B cell activation by parasite mitogens contributes to the mechanisms leading to parasite evasion and persistence in the mammalian host. It has also been demonstrated that TcPRAC is a potent B cell mitogen released by the infective forms of the parasite. The TcPRAC inhibition by pyrrole carboxylic acid induces a total loss of TcPRAC B cell mitogenic ability.

It has also been shown that the overexpression of TcPRACA and TcPRACB genes by mutant parasites are able to confer to these mutants a better invasion ability of host cells in vitro. This corresponds with the inability of parasites to survive if these TcPRAC genes are inactivated by genetic manipulation. In addition, the immunization of mice with sub-mitogenic doses of TcPRAC, or with appropriate TcPRAC-DNA vector vaccine preparations, was shown to trigger high levels of specific antibody responses directed to TcPRAC and high levels of immunoprotection against an infectious challenge with live Trypanosoma cruzi.

Altogether, these data suggest that TcPRAC enzyme isoforms are essential elements for parasite survival and fate and also support that parasite proline racemase is a good target for both vaccination and chemotherapy. In fact, the addition of pyrrole carboxylic acid at TcPRAC neutralizing doses to non-infected monkey cell cultures do not interfere with cellular growth. Besides, the utilization of a proline racemase inhibitor in humans would be a priori possible since the absence of the two critical active site cysteine residues (Cys 330 and Cys 160) for the PRAC enzyme activity has been observed in the single sequence that displays some peptide homologies with TcPRAC that was identified by blasting the Human Genome available data with the TcPRAC gene sequence.

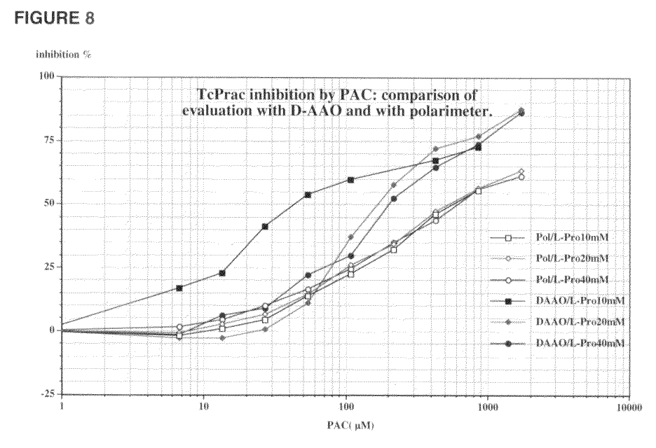

As observed by data mining using TcPRAC gene sequences, it has been possible to identify putative proline racemases in other microrganisms of medical and agricultural interest. As can be seen in FIG. 8, the presence of MI, MII and most particularly MIII indicates the potentiality of those proteins to be functional proline racemases. On the one hand, it can be observed that critical residues necessary for the enzyme activity are displayed in those sequences and, on the other hand, that the open reading frames (ORF) are highly homologous to the ORF of the parasite PRAC.

In order to search for putative molecules that could be used as inhibitors of TcPRAC, or other proline racemases, it would be necessary to develop a microtest able to specifically reveal the inhibition of proline racemization performed by TcPRAC and consequently the blockage of a given proline stereoisomer generation. For instance, this could be done by analysing the ability of any potential inhibitory molecule to hinder the generation of D-proline in a reaction where L-proline is submitted to TcPRAC enzymatic activity.

At present, the available analyses to detect D- (or L-) amino acids are very challenging and methods to differentiate L-stereoisomers from D-stereoisomers are time-consuming, i.e. gas chromatography, thin layer chromatography using chiral plates, high-performance capillary electrophoretic methods, HPLC, and some enzymatic methods. Some of those techniques also require the use of columns and/or heavy equipment, such as polarimeters or fluorescence detectors.

With the aim of developing a simple test that is useful to rapidly screen putative inhibitors of TcPRAC, TcPRAC constructs allowing for the production of high amounts of the recombinant active enzyme were used together with the knowledge of a specific inhibitor of proline racemases (pyrrole carboxylic acid, PAC) to develop a medium/high throughput microplate test that can be used to easily screen a high number of inhibitor candidates (i.e. 100-1000). Such a test is based on calorimetric reactions that are certainly a simpler alternative to polarimetry and other time-consuming tests. Thus, the evaluation of light deviation of L- or D-proline enantiomers by a polarimeter to quantify the inhibition of proline racemization to test such an elevated number of molecules is impracticable, offers a low sensibility, and would require greater amounts of reagents as compared to a microplate test that would additionally be of an affordable price.

Accordingly, this invention is based on the detection of D-amino acids originated through racemization or epimerization of L-amino acids, in the presence or in the absence of known concentrations of racemase and epimerase inhibitors as positive and negative controls of enzyme activity, respectively. For that purpose, this invention utilizes another enzyme, D-amino acid oxidase (D-AAO), that has the ability to specifically oxidize D-amino acids in the presence of a donor/acceptor of electrons and yield hydrogen peroxide. The advantage of this strategy is that hydrogen peroxide can be classically quantified by peroxidase in a very sensitive reaction involving ortho-phenylenediamine, for example, ultimately offering a chromogenic reaction that is visualized by colorimetry at 490 nm.

Since D-amino acid oxidase reacts indiscriminately with any “D-amino acid,” and not with their L-stereoisomers, such a test is not only helpful to identify racemase and epimerase inhibitors, but also applicable, if slightly modified, to detect any alterations in levels of free D-aa in various fluids to make a diagnosis of some pathogenic processes.

### I-Basics for a D-Amino-Acid Quantitative Test

The following method of the invention allows detection and quantitation of D-Amino acids. A first reaction involves a D-amino-oxidase. This enzyme specifically catalyses an oxidative deamination of D-amino-acids, together with a prosthetic group, either Flavin-Adenin-Dinucleotide (FAD) or Flavin-Mononucleotide (FMN), according to the origin of the Enzyme. (Obs. FAD if the enzyme comes from porcine kidney).

The general reaction is as follows:

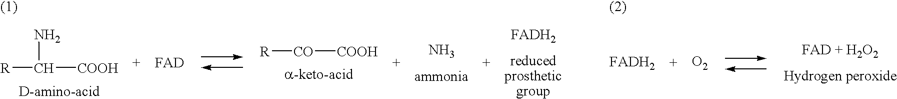

In (1), the D-amino acid is deaminated and oxidized, releasing ammonia and the reduced prosthetic group. If the amino group is not a primary group, the amino group remains untouched and no ammonia is released.
In (2), the reduced prosthetic group reduces oxygen, and generates hydrogen peroxide. Either a catalase or a peroxidase can decompose hydrogen peroxide.
A catalase activity is written as:

2H₂O₂>2H₂O+O₂ (O═O)

### Oxygen

whereas a peroxidase activity is

H₂O₂+HO—R′—OH>2H₂O═O═R′═O

wherein R′ is any carbon chain

Thus, detection of hydrogen peroxide can be done with the use of catalase and a reagent sensitive to oxygen such as by destaining reduced methylene blue for instance with oxygen or with the use of peroxidase with a change in color of the reagent indicated by:

HO—R′—OH→O═R′═O

II—Application of Such a Test for Evaluating the T. Cruzi Racemase Activity and the Inhibition of this Racemase.

### II-1—Test for Racemase Activity

The T. cruzi racemase activity converts reversibly L-Pro into D-Pro. Since these two forms can induce polarized light deviation, this conversion can be measured by optical polarized light deviation. But the presence of the D-form allows also the use of D-amino-acid oxidase in order to assess the amount of D-Proline in racemase kinetics. In this test the following reactions are involved:

### 1) Proline-Racemase Activity.

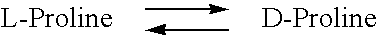

### 2) D-amino-Acid Oxidase

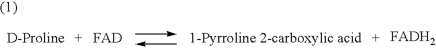

(Obs: There is no ammonia formed in the case of Proline, because the nitrogen of Proline is involved in a secondary amine.)

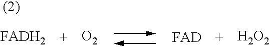

3) Detection of Hydrogen Peroxide with Peroxidase

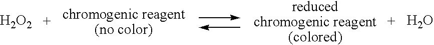

The chromogenic reagent can be, for example, orthophenylenediamine (OPD), or 3,3′,5,5′ tetramethyl benzidine (TMB), or 5-aminosalicylic acid (ASA).

These reactions can be carried out using the following exemplary, but preferred, materials and methods.

### II-1-1—Materials

| Materials | Comments |
|---|---|
| Proline-racemase (TcPRAC) (1 mg/ml Stock) | |
| L-Proline, Sigma, Ref.P-0380 (1M Stock) | An equimolar of D- and L-Proline is made by |
| D-Proline, Aldrich, réf. 85 891-9 (1M Stock) | mixing equal volumes of 2M D-Proline with 2M L- |
| | Proline |
| Orthophenylenediamine (OPD) Sigma refP-8287 | 10 mg tablets. Extemporaneously used as a |
| lot 119H8200 | 20 mg/ml stock solution in water. |
| D-AAO from swine kidney (Sigma) ref. A-5222 lot | Powder dissolved into 1 ml Buffer* + 1 ml 100% |
| 102K1287 | glycerol. The resulting activity is 50 U/ml. Stored at |
| | −20° C. |
| Horse radish peroxidase (HRP) Sigma ref P8375 | Powder dissolved into 2.5 ml Buffer* + 2.5 ml 100% |
| lot 69F95002 | glycerol. The resulting activity is 5042 U/ml.Stored |
| | at −20° C. |
| Sodium acetate 0.2M Ph 6.0 | |
| Flavine-adenine-dinucleotide (FAD) (Sigma) ref. | Stock solution of 10⁻¹ M in water. Stored at −20° C. |
| F-6625 | Used as a 10⁻³ M sub-stock solution. |
| Sodium pyrophosphate (Pop) 0.235M | Not soluble at a higher concentration. Must be |
| | stored at 4° C. and gently heated before use in |
| | order to solubilize crystals which may occur. |
| Buffer* = 10 ml of 0.2M sodium acetate buffer | The final pH is 8.3. |
| pH 6.0 + 680 μl 0.235M Pop | |
| Microplates (96 wells) | With adhesive coverlid |
| ELISA reader for microplates | With a wavelength filter at 490 nm for OPD |
| | substrate. |

### II-1-2—Methods

II-1-2.1—Racemisation in microplates:

(1) The volumes are indicated for a single well, but duplicates are mandatory. Leave enough raws of the microplate empty for standard and controls to be used in further steps. Distribute the following volumes per well reactions:

a) without inhibitor (Vol=QS 81 μl)

| | | | | | | | | |
|---|---|---|---|---|---|---|---|---|
| TcPRAC 1 mg/ml | 2 | μl | 2 | μl | 2 | μl | 2 | μl |
| L-Proline 0.1M | 32 | μl | 16 | μl | 8 | μl | 4 | μl |
| Proline Final | (40 | mM) | (20 | mM) | (10 | mM) | (5 | mM) |
| concentration | | | | | | | | |
| Sodium acetate | 47 | μl | 63 | μl | 71 | μl | 75 | μl |
| buffer 0.2M | | | | | | | | |
| pH6 | | | | | | | | |

b) with inhibitor (Vol=QS 81 μl)

A range of concentrations between 5 mM and 1 mM can be planned for the inhibitor. It should be diluted in sodium acetate buffer 0.2 M pH 6.0. Hence, the volume of inhibitor is subtracted from the volume of buffer added in order to reach a final volume of 81 μl. For instance, 50% inhibition of racemisation of 10 mM L-proline is obtained with 45 μM Pyrrole carboxylic acid (PAC, specific inhibitor of proline racemase), when 36.5 μl PAC+44.5 μl buffer are used (see results in FIG. 8). Table VII is provided for 10 mM L-Proline as a substrate.

TABLE VII

| | | | | | | | | | | | | | | | | | | | | |
|---|---|---|---|---|---|---|---|---|---|---|---|---|---|---|---|---|---|---|---|---|
| TcPrac 1 mg/ml | 2 | μl | 2 | μl | 2 | μl | 2 | μl | 2 | μl | 2 | μl | 2 | μl | 2 | μl | 2 | μl | 2 | μl |
| L-Proline 0.1 M | 8 | μl | 8 | μl | 8 | μl | 8 | μl | 8 | μl | 8 | μl | 8 | μl | 8 | μl | 8 | μl | 8 | μl |
| PAC | 0 | μl | 5.4 | μl | 11 | μl | 22 | μl | 43 | μl | 9 | μl** | 17 | μl** | 35 | μl** | 69 | μl** | 14 | μl*** |
| 0.1 mM/1 mM**/ | | | | | | | | | | | | | | | | | | | | |
| 10 mM*** | | | | | | | | | | | | | | | | | | | | |
| Final concentration (μM) | 0 | | 6.7 | | 13.5 | | 27 | | 54 | | 107 | | 214 | | 429 | | 858 | | 1715 | |
| Sodium acetate buffer | 71 | μl | 65.6 | μl | 60 | μl | 49 | μl | 28 | μl | 62 | μl | 54 | μl | 36 | μl | 2 | μl | 57 | μl |
| 0.2 M pH6 QS 81 μl | | | | | | | | | | | | | | | | | | | | |

(2) Cover the microplate with an adhesive coverlid and leave for 30 nm at 37° C.
(3) At the end of racemisation, 5.5 μl of 0.235M Pop are added in each reaction well of the microplate in order to shift pH from pH6.0 to pH 8.3.

### II-1-2.1-2—Quantitation of Formed D-Proline: Standards and Controls.

(1) Prepare standard and controls:

Standard: An equimolar mixture of L- and D-Proline is used as a standard in a range from 0.05 mM to 50 mM (final concentration in the assay). It is used for assessing the amount of D-Proline formed after racemization. The standard range is made in microtubes, as follows:

In tube 1, mix Proline and buffer according to the described proportions.

Then, add 500 μl of the obtained mixture to 500 μl of buffer in next tube, and so on.

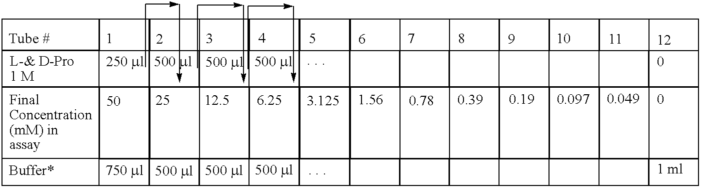

Negative control is prepared in an other microtube, as follows:

| | |
|---|---|
| L-Proline (1M) | 200 μl |
| Buffer* | 800 μl |
| Final concentration | 40 ml |

Blank = Buffer*.

(2) Dispense in the empty wells of the microplate (see step II-1-2.1):

| | |
|---|---|
| Buffer* | 67 μl |
| Standard dilutions | 20 μl |
| or negative control | |

Obs: For the blank dispense 87 μl of Buffer* only

(3) Prepare a mixture containing the enzymes (D-AAO/HRP Mix), as follows:
The amounts are given for one well, provided that the final volume will be 100 μl with the racemase products or the substrate:

| | For 13 μl: |
|---|---|
| Buffer* | 6.5 μl |
| D-AAO 50 U/ml | 1.7 μl |
| OPD (20 mg/ml) | 2.5 μl |
| HRP 5000 U/ml | 0.75 μl |
| FAD 10⁻³M (4.5 μl 10⁻¹M + 446 μl buffer) | 1.5 μl |

This mixture is kept in the ice until use.
(4) The quantitation reaction starts when 13 μl of D-AAO/HRP mix is added to the reaction well.
(5) The microplate is covered with an adhesive coverlid and it is left in the dark at 37° C. between 30 nm and 2 hours. The reaction can be monitored by eye whenever a color gradient matches the D-amino acid concentration of the standard dilutions.
(6) The microplate is read with a microplate spectrophotometer using a filter at 490 nm.

### EXAMPLE 14

### D-AOO Microplate Test is More Sensitive Than D-Amino Acid Detection by Detection in Polarimeter

In order to compare the D-Proline quantitation by polarimeter and by D-amino-oxidase/HRP a comparison was performed between the two tests using different concentrations of L-proline and different concentrations of PAC, the specific inhibitor of proline racemases. FIG. 8 shows the percent of racemisation inhibition of different L-proline concentrations (ranging from 10-40 mM) using the D-AAO (D-AAO/L-) microtest as compared to conventional detection using a polarimeter (Pol/L-).

With the polarimeter, there seems to be no difference of PAC inhibition of TcPRAC with the three concentrations of L-Proline. Therefore, 50% inhibition is obtained with 1 mM PAC, whether 10 mM or 40 mM L-Proline is used. In contrast, when using D-AAO/HRP test, it can be seen that inhibition by PAC is somewhat higher with a low concentration of L-Proline (10 mM for example) than with an increased one (20 mM or 40 mM). Therefore, 50% inhibition is obtained
with 50 μM PAC when 10 mM L-Proline is used,
with 170 μM PAC when 20 mM L-Proline is used and
with 220 μM PAC when 40 mM L-Proline is used.

In conclusion, D-AAO/HRP evaluation is more sensitive since it can discriminate PAC inhibition at a lower concentration than evaluation with the polarimeter. Furthermore, inhibition is logically conversely proportional to L-Proline concentration, which can be assessed with the D-AAO/HRP method, but not with the polarimeter measurement. Such a test is useful for the screening of new inhibitors of TcPRAC in a medium/high throughput test.

A preferred technological platform to perform the above test and to select appropriate inhibitors contains at least the following products:
L-Proline, D-Proline, a proline-racemase
A peroxidase, a substrate of a peroxidase
A D-amino-acid oxidase
And optionally a battery of potential inhibitory molecules.

### EXAMPLE 15

### L-Proline Inhibits D-Amino-Oxidase Activity

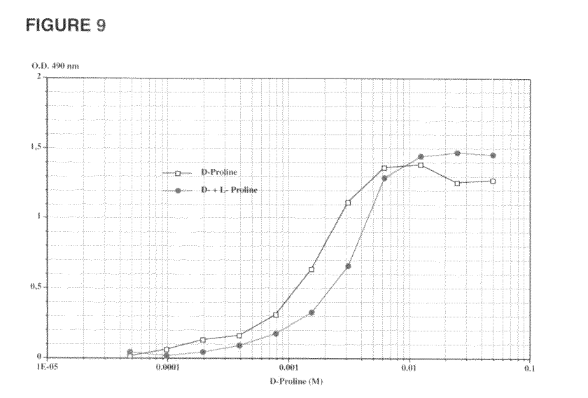

FIG. 9 shows the comparison of D-AAO/HRP reaction using D-Proline alone or an equimolar mixture of D- and L-Proline as standard. It can be seen that the amount of D-Proline required to obtain a given optical density is higher when a mixture of L- and D-Proline are used as compared to a standard using D-proline alone. Since Proline-racemase activity ends when both L- and D-Proline are in equal amounts, it was also adequate to use an equimolar mixture of both enantiomers of Proline as standard for D-Proline determination.

### EXAMPLE 16

### PAC Does Not Interfere with DAAO/HRPactivity

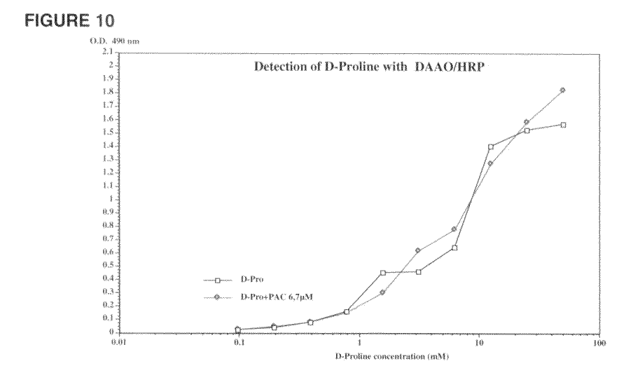

FIG. 10 shows optical density at 490 nm as a function of D-proline concentration under the following conditions.

Conditions in μl wells,

[D-Proline]range between 0.1 mM and 40 mM

| | |
|---|---|
| [D-AAO] | 0.89 U/ml |
| [HRP} | 37.5 U/ml |
| [OPD] | 0.5 U/ml |
| [FAD] | 1.5 × 10⁻⁵M |

Buffer*

The presence of PAC does not influence DAAO/HRP reaction.

### EXAMPLE 17

### A Medium/High Throughput Test Using the D-AAO Microplate Test

Table VIII is an Example of a medium/high throughput test using the D-AAO microplate test.

Blue: D-proline standard (column 1)
Green: Positive control of racemization using 10 mM substrate (column 2, line A and B)
Orange: control for inhibition of racemization reaction by PAC using 10 mM substrate (column 2, line C and D)
Blank 1: mix with racemase (column 2, line E)
Blank 2: mix without racemase (column 2, line F)
Yellow: Negative control for specificity of (without racemase+40 mM L-proline) (column 2, line G and H)
Other wells: with Inhibitors (T1, T2, T3, . . . T40): in duplicates

TABLE VIII

| | 1 | | | | | | | | | | | |
|---|---|---|---|---|---|---|---|---|---|---|---|---|
| | D-Pro | | | | | | | | | | | |
| | (mM) | 2 | 3 | 4 | 5 | 6 | 7 | 8 | 9 | 10 | 11 | 12 |
| A | 10 | L-Pro | T1 | T2 | T3 | T4 | T5 | T6 | T7 | T8 | T9 | T10 |
| | | L-Pro | ″ | ″ | ″ | ″ | ″ | ″ | ″ | ″ | | |
| | | L-Pro + PAC | T11 | T12 | T13 | T14 | T15 | T16 | T17 | T18 | T19 | T20 |
| | | L-Pro + PAC | ″ | ″ | ″ | ″ | ″ | ″ | ″ | ″ | ″ | ″ |
| | | Blanc 1 | T21 | T22 | T23 | T24 | T25 | T26 | T27 | T28 | T29 | T30 |
| | | Blanc 2 | ″ | ″ | ″ | ″ | ″ | ″ | ″ | ″ | ″ | |
| | | L-Pro | T31 | T32 | T33 | T34 | T35 | T36 | T37 | T38 | T39 | T40 |
| H | 0.07 | L-Pro | ″ | ″ | ″ | ″ | ″ | ″ | ″ | ″ | | |

### EXAMPLE 18

### Application of Such a Test for General Detection of D-Amino Acids in Samples

The use of a microplate test based on D-amino-acid oxidase together with a peroxidase, such as horseradish peroxidase, can be used to detect and quantitate any D-amino acid in any biological or chemical sample. For example, since D-amino acids are described to be involved in several pathological processes or neurological diseases, such as Alzheimer disease, Parkinson, or renal diseases, their detection can be an important marker or parameter for the diagnosis and the follow-up of these pathologies. This technology can be also extended to the detection and quantification of D-amino acids in eukaryotic organisms, such as plants or fungi, and in bacteria.

The D-AAO/HRP test described here above can also be used for this purpose with slight modifications. For that purpose, the racemase reaction step should be skipped and the microplate test should start straightforward at the II-1-2, 1-2 step described above with the following remarks:

1) Standard: It should not be an equimolar mixture of D- and L-amino acid, but rather a serial dilution of D-Amino acids. The choice of amino acid is made according to the interest of the D-amino acid under investigation. The final volume in wells should be of 87 μl.

2) Negative control: It is made with the L-enantiomer of the D-amino acid under investigation. The final volume should be 87 μl.

3) Blank: It is made with 87 μl buffer*. (See paragraph 11.1.1 Materials.)

4) Samples: The samples to be tested should be adjusted to pH 8,3 with buffer* and their final volumes should be of 87 μl per well.

Obs: Standards, negative controls, samples to test and blanks should be made in duplicates. They are dispensed into the wells of the microplate.

5) Then, the procedure follows steps 3) to 6), as above.

Several D-amino acids and their L-counterparts have been tested using the microplate test described above. Tables IX and X show that D-forms of Tyrosine, Valine, Threonine, Glutamic acid, Lysine and Tryptophane are indeed substrates for the D-AA0/HRP and are detected by the test, as described for D-Proline. The results also show that no L-amino acid is detected by such a methodology.

TABLE IX

| | | | | | | | | | | | | | |
|---|---|---|---|---|---|---|---|---|---|---|---|---|---|
| A | Blank | 49.5 | 24.75 | 12.37 | 6.19 | 3.09 | 1.55 | 0.77 | 0.39 | 0.19 | 0.09 | 0.05 | D-pro |
| B | Blank | 49.5 | 24.75 | 12.37 | 6.19 | 3.09 | 1.55 | 0.77 | 0.39 | 0.19 | 0.09 | 0.05 | mM |
| C | Blank | L-Tyr | L-Val | L-Thr | L-Glu | L-Lys | L-Try | | | | | | |
| D | Blank | 12.5 | 12.5 | 12.5 | 12.5 | 12.5 | 12.5 | mM | | | | | |
| E | Blank | D-Tyr | D-Val | D-Thr | D-Glu | D-Lys | D-Try | | | | | | |
| F | Blank | 6.25 | 6.25 | 6.25 | 6.25 | 6.25 | 6.25 | mM | | | | | |

Optical densities at 490 nm obtained after D-AAO reaction. (raw OD data).

TABLE X

| | | | | | | | | | | | | | |
|---|---|---|---|---|---|---|---|---|---|---|---|---|---|
| A | 0.105 | 1.961 | 1.757 | 1.814 | 1.983 | 1.716 | 1.234 | 0.809 | 0.496 | 0.308 | 0.213 | 0.173 | D-pro |
| B | 0.118 | 2.004 | 1.885 | 1.976 | 1.949 | 1.879 | 1.221 | 0.824 | 0.504 | 0.32 | 0.215 | 0.159 | mM |
| C | 0.123 | 0.193 | 0.135 | 0.124 | 0.131 | 0.125 | 0.131 | L- | | | | | |
| D | 0.125 | 0.141 | 0.129 | 0.128 | 0.141 | 0.131 | 0.138 | L- | | | | | |
| E | 0.120 | 1.317 | 1.683 | 0.215 | 0.147 | 0.243 | 0.615 | D- | | | | | |
| F | 0.105 | 0.991 | 1.612 | 0.157 | 0.116 | 0.157 | 0.662 | D- | | | | | |

Template of microplate is where a serial dilution of D-Proline (mM) was made as positive control of the D-AAO reaction. Blank wells containing buffer* are shown. Different L- and D-amino acids were tested, namely Tyrosine (Tyr), Valine (Val), Threonine (Thr), Glutamic acid (Glu), Lysine (Lys) and Tryptophan (Try). To highlight the sensitivity of the D-AAO microtest, higher concentrations of L-enantiomers (12.5 mM) were used in the reactions as compared to the concentrations used for D-enantiomers (6.25 mM):

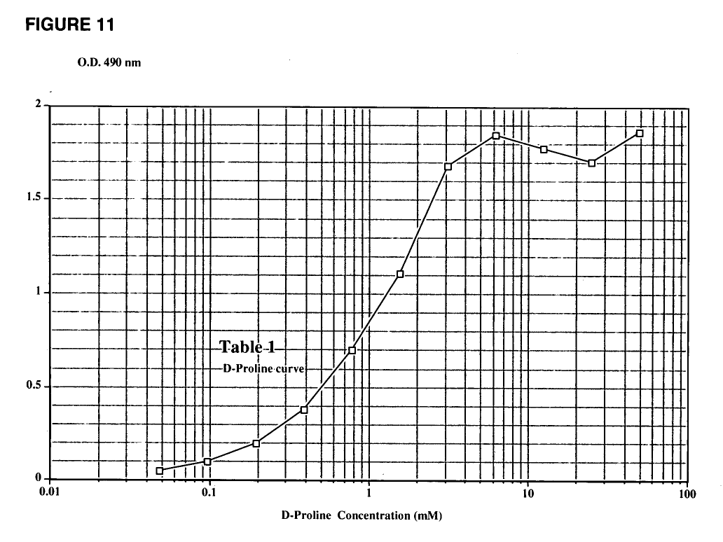

FIG. 11 is a Graph obtained with the serial dilutions of D-proline, as positive reaction control Obs: OD of wells (−) average of OD obtained from blank wells.

A preferred platform to search and quantitate the presence of a D-Amino acid in samples contains at least the following products:
A D-amino acid,
A peroxidase and a substrate of a peroxidase
A D-amino-acid oxidase
And optionally, a L-amino acid enantiomer, as control.

This invention relates to a method for screening a molecule, which can modulate a racemase activity, wherein the method comprises:
(A) modulating a racemase activity by means of a molecule being tested in the presence of an equimolar mixture of a L- and D-amino acid and of a racemase to be modulated;
(B) oxidatively deaminating the D-amino acid generated in step (A) by means of a D-amino oxidase in a prosthetic group; and
(C) detecting the hydrogen peroxide generated by the oxidative deamination;
   wherein modulation of the hydrogen peroxide is indicative of the capability of the tested molecule to modulate racemase activity. Preferably the molecule inhibits racemase activity, and more preferably the racemase is a proline racemase, for example, Tripanosoma cruzi proline racemase. A molecule identified by a method is also part of this invention.

Further, this invention relates to technological platform and all reagents and devices necessary to perform the methods of the invention. The technological platform comprises:
a) L-amino acid, D-amino acid, and a racemase;
b) a peroxydase and a substrate of a peroxydase, or a catalase and a reagent sensitive to oxygen;
c) a D-amino acid oxidase; and
d) optionally, one or more molecules to be screened for inhibitory activity of said racemase.

Preferably, the racemase is a proline racemase and the L-amino acid and D-amino acid are L-proline and D-proline, respectively.

Preferably, a molecule inhibits a proline racemase containing a subsequence selected from the SEQ ID NO: 1, 2, 3, 4, or 130.

### EXAMPLE 19

### In Silico Gene Selection of Homologous PRAC Genes

The discovery of novel microbial genes and metabolic proteins through genome mining has proven to be a promising approach for identifying potential candidates for drug discovery and therapy against infections. In order to identify additional PRAC homologues from other pathogens, genomic databases were Blast-screened using TcPRAC sequence (AF195522, NCBI, E.C.5.1.1.4) and PRAC motif III*. Default settings for Blast were used. Unrooted trees and alignments were obtained with the ClustalW program.

The Blast searches of NCBI and Swiss-Prot/TrEMBL databases with full-length TcPRAC sequences resulted in 184 hits of which, 111 possessed the MIII* (DRSPCGXGXXAXXA) motif. Of those, 62 hits were directly annotated as “PRAC,” without previous validation of the enzymatic activity. The present invention reveals that the MIII* and MCGH motifs (25), encompassing the TcPRAC Cys₃₀₀ and Cys₁₃₀ crucial residues respectively, are consistently present in 92 sequences. A collection of 15 sequences was selected for further studies according to sequence identities with TcPRAC, the conservation or not of homologous Cys₁₃₀ and Cys₃₀₀, and the recognized pathogenic importance of the microbial genomes.

As summarized in Table XI, homologous genes from different pathogen strains, annotated as “putative PRAC,” “PRAC,” or “unknown” proteins, display 29 to 56% homology with TcPRAC and present either a conservation of the couple of catalytic Cys residues or replacements of one or both Cys positions by Ser and/or Thr residues. A comparison between Brucella spp sequences and the previously characterized TcPRAC and CsPRAC is of note. Therefore, from the two available homologous sequences for each Brucella specie only one meets the requirements for PRAC activity and presents both key Cys residues, the other presenting Ser and Thr substitutions.

TABLE XI
Database collection from in silico searches and complementary
information on selected sequences

| | | | | | TcPRAC | |
|---|---|---|---|---|---|---|
| | | | | | Homology | |
| Pathogen | Disease | Acces. nb^{†} | MCGH^{§} | MIII*^{§} | (%) | Annot.^{‡} |
| Trypanosoma cruzi | Chagas' disease | Q868H8 | C | C | 100 | PRAC |
| CL Brener | | | | | | |
| Bacillus anthracis Ames | Anthrax | Q81PH1 | C | C | 40 | Put. PRAC |
| | | Q81UH1 | C | C | 40 | |
| Brucella abortus 9-941 | Brucellosis | Q57B94 (1) | C | C | 40 | |
| | | Q57F22 (2) | S | T | 29 | |
| Brucella melitensis 16M | | Q8YJ29 | C | C | 40 | PRAC |
| | | Q8YFD6 | S | T | 29 | |
| Brucella suis 1330 | | Q8FYS0 | C | C | 40 | Put PRAC |
| | | Q8G2I3 | S | T | 29 | |
| Burkholderia cenocepacia HI2424 | Pneumonia, sepsis | A0AZQ0 | C | C | 29 | PRAC |
| | | A0B0B8 | C | T | 37 | |
| Burkholderia pseudomallei K96243 | Melioidosis | Q63NG7 | C | C | 34 | Hyp. prot |
| Clostridium difficile 630 | Nosocomial diarrhoea | Q17ZY4 | C | C | 56 | Put. PRAC |
| Pseudomonas aeruginosa PAO1 | Pneumonia, sepsis | Q9I476 | C | C | 33 | Hyp. prot |
| | | Q9I489 | S | C | 30 | |
| Vibrio parahaemolyticus O3:K6 | Diarrhoea | Q87Q20 | C | C | 37 | PRAC |

Table XI Legend:
Sequences were obtained by blasting TcPRAC (Q868H8) against Swiss-Prot/TrEMBL or NCBI databases.
^{†}Swiss-Prot accession number;
^{§}MCGH and MIII* motifs are minimal peptide sequences encompassing TcPRAC catalytic Cys residues (Cys₁₃₀ and Cys₃₀₀);
^{‡}Related annotation from blast searches.

Table XII presents an updated version of Table V with the results of the SWISS-PROT and TrEMBL database screen using motifs I to III (MI, MII and MIII). MI corresponds to [IVL][GD]XHXXG[ENM]XX[RD]X[VI]XXG, MII to [NSM][VA][REP][AS][FY]X(13,14)[GK]X[IVL]XXD[IV][AS][YWF]GGX[FWY], MIII to DRSPXGXGXXAXXA and MIII* to DRSPCGXGXXAXXA.

TABLE XII
SWISS-PROT and TrEMBL databases screening
using PROSITE motifs

| | | | Motifs | | | |
|---|---|---|---|---|---|---|
| | | | | M | M | M |
| Organism | Seq | Access. nb | M I | II | III | III* |
| Agrobacterium tumefaciens | 1 | Q8UIA0 | + | + | + | + |
| Agrobacterium tumefaciens | 2 | Q8U6X2 | − | − | + | − |
| Agrobacterium tumefaciens | 3 | Q8U8Y5 | − | − | + | − |
| [symbol] | 1 | Q8YJ29 | − | + | + | + |
| Brucella melitensis | 2 | Q8YFD6 | + | − | + | − |
| [symbol] | | Q9L4Q3 | − | + | + | + |
| Homo sapiens | 1 | Q96EM0 | + | + | + | − |
| Homo sapiens | 2 | Q96LJ5 | + | + | + | − |
| Mus musculus | 1 | Q9CXA2 | + | + | + | − |
| Mus musculus | 2 | Q99KB5 | + | + | + | − |
| [symbol] | 1 | Q9I476 | − | + | + | + |
| [symbol] | 2 | Q9I489 | − | − | + | + |
| Rhizobium loti | 1 | Q98F20 | − | + | + | + |
| Rhizobium loti | 2 | Q988B5 | + | + | + | − |
| Rhizobium meliloti | 1 | Q92WR9 | − | − | + | − |
| Rhizobium meliloti | 2 | Q92WS1 | − | + | + | + |
| Streptomyces coelicolor | | Q93RX9 | + | − | + | + |
| [symbol] | 1 | Q9NCP4 | + | + | + | + |
| [symbol] | 2 | [symbol] | + | + | + | + |
| Xanthomonas axonopodis | 1 | Q8PJI1 | − | + | + | + |
| Xanthomonas axonopodis | 2 | Q8PKE4 | − | − | + | + |
| Xanthomonas campestris | | Q8P833 | − | + | + | + |
| Other sequences | | | | | | |
| [symbol] | 1 | Q81UH1 | + | − | + | + |
| [symbol] | 2 | Q81PH1 | − | − | + | + |
| Bacillus cereus | 1 | Q81HB1 | + | − | + | + |
| Bacillus cereus | 2 | Q81CD7 | − | − | + | + |
| [symbol] | 1 | Q8FYSO | + | + | + | + |
| Brucella suis | 2 | Q8G213 | + | − | + | − |
| Chromobacterium violaceum | | Q7NU77 | + | + | + | + |
| Photorhabdus luminescens | | Q7N4S6 | + | + | + | + |
| Pseudomonas putida | | Q88NF3 | + | + | + | + |
| Rhodopirella baltica | | Q7UWF3 | − | − | + | + |
| Streptomyces avermitilis | | Q82MDO | + | − | + | + |
| [symbol] | | Q87Q20 | + | + | + | + |

Table XII Legend:
sequences underlined in dotted lines are characterized true hydroxyproline epimerases;
sequences double underlined are characterized proline racemases;
sequences in strikethrough do not present any PRAC or HyPRE activities;
Access.nb, SWISS-PROT accession number of the sequence;
seq, number of sequences according to FIG. 6 of reference 2;
+ and −, presence or absence, respectively, of hit using the corresponding motif.

Table XIII presents an updated version of Table VI with the results of the SWISS-PROT and TrEMBL database screen using motifs I to III (MI, MII and MIII). MI corresponds to [IVL][GD]XHXXG[ENM]XX[RD]X[VI]XXG, MII to [NSM][VA][REP][AS][FY]X(13,14)[GK]X[IVL]XXD[IV][AS][YWF]GGX[FWY], MIII to DRSPXGXGXXAXXA and MIII* to DRSPCGXGXXAXXA. R1, R2, and R3 are additional elements allowing the discrimination between PRAC and HyPRE. Sequences with double underlines are characterized proline racemases; sequences underlined in dotted lines are characterized true hydroxyproline epimerases; sequences in strikethrough do not present any PRAC or HyPRE activities; Access nbs, TIGR, EMBL, SwissProt or SANGER are accession numbers of the sequence; + and − indicate the presence or absence respectively of the corresponding motif.

TABLE XIII

| | | | | | | M III* | MCGH | R1 | R2 | R3 |
|---|---|---|---|---|---|---|---|---|---|---|
| Organism | Accession number | Database | M I | M II | M III | Cys³³⁰ | Cys¹⁶⁰ | Phe | His | XLA |
| Aspergillus fumigatus | Af0787f05.plc | TIGR | + | − | + | − | + | | | |
| [symbol] | TIGR 5085 | TIGR | + | + | + | + | ? | | | |
| 70 | Q81PH1 seq 1 | SwissProt | + | + | + | + | + | − | − | − |
| [symbol] | Q81UH1 seq 2 | SwissProt | + | + | + | + | + | − | − | − |
| Bacillus cereus ATCC14579 | AE017007 | EMBL | + | + | + | + | + | | | |
| [symbol] | Q57B94 | SwissProt | + | + | + | + | + | − | + | + |
| Brucella abortus | Q57F22 | SwissProt | + | + | + | − | − | − | − | − |
| [symbol] | Q8YJ29 | SwissProt | + | + | + | + | + | − | + | + |
| Brucella melitensis | Q8YFD6 | SwissProt | + | + | + | − | − | − | − | − |
| [symbol] | Q8FYS0 | SwissProt | + | + | + | + | + | − | + | + |
| [symbol] | Q8G2I3 | SwissProt | + | + | + | − | − | − | − | − |
| [symbol] | A0B0B8 | SwissProt | + | − | + | − | + | − | − | − |
| Burkholderia mallei | contig: 33162: b mallei | TIGR | + | + | + | + | + | | | |
| [symbol] | Q63NG7 | SwissProt | + | + | + | + | + | − | + | + |
| Clostridium botulinum | SANGER 36826 | Sanger | + | + | + | + | + | | | |
| [symbol] | Q17ZY4 | SwissProt | + | + | + | + | + | + | − | − |
| [symbol] | Q9L4Q3 | SwissProt | + | + | + | + | + | + | − | − |
| [symbol] | LM16BINcontig2054 | Sanger | ? | + | + | + | + | | | |
| [symbol] | AE016778 | EMBL | + | + | + | + | + | | | |
| [symbol] | Q91476 | SwissProt | + | + | + | + | + | − | + | + |
| [symbol] | Q91489 | SwissProt | + | − | + | + | − | − | − | − |
| [symbol] | UTHSC 1063 | UTHSC | + | ? | − | − | + | | | |
| [symbol] | TbKIX28b06.qlc | Sanger | ? | + | ? | ? | + | | | |
| [symbol] | TbKIX28b06.plc | Sanger | ? | + | ? | ? | + | | | |
| [symbol] | Q868H8 | swissProt | + | + | + | + | + | + | − | − |
| [symbol] | Q4DA80 | SwissProt | + | + | + | + | + | + | − | − |
| [symbol] | Tviv1929b09.p1k—8 | GeneDB | + | + | + | + | ? | + | + | − |
| [symbol] | congo208e06 | Sanger | ? | + | + | + | ? | | | |
| [symbol] | Q87Q20 | SwissProt | + | + | + | + | + | − | + | − |

### EXAMPLE 20

### Functional PRAC and Hydroxyproline Epimerases of Pathogenic Bacteria

The function of 12 gene products and their ability to interconvert Pro residues was addressed. Purified DNA was obtained from B. anthracis (strain 9131), C. difficile (strain VPI10463), V. parahaemolyticus (CNRVC 010089), B. abortus (strain 544), B. melitensis (strain 16M), B. suis (strain 130) and B. pseudomallei (strain K96243). DNA was extracted from bacterial pellets of B. cenocepacia (strain J2315) and P. aeruginosa (strain PAK) with the DNA tissue culture extraction kit (Qiagen).

Forward and Reverse primers were designed based on TcPRAC sequence toward specific sequences of the genes of interest (Table XIV). Bacterial PCR products were purified by QuickPCR® Qiaprep kit (Qiagen) and cloned into BamHI/EcoRI or BamHI/NcoI sites of pET28b (Novagen/Merck) using Rapid Ligation Kit® (Roche). E. coli DH5α cells were transformed with empty or ligated plasmids. Plasmids were extracted with the Qiaprep® Spin Miniprep kit (Qiagen) from bacterial pellets from individual colony cultures and sequenced (Genome Express, Meylan/France). Sequences, ORFs, and the presence of C-terminal 6×-His Tag were verified. E. coli BL21 (DE3) cells were transformed with ligated plasmids. Recombinant proteins were purified as described (3).

TABLE XIV
Primers used for the production of recombinant proteins and site-directed muta genesis.

| | Swill-prot | Forward and Reverse primers for |
|---|---|---|
| Pathogen strain | AC | recombinant protein production |
| Trypanosoma cruzi | Q868H8 | F: 5′-CTCTCCCATGGGGCAGGAAAAGCTTCTG-3′ |
| CL Brener | | R: 5′-CTGAGCTCGACCAGAT(CA)TACTGC-3′ |
| Bacillus anthracis Ames | Q81PH1 | F: 5′-TCATGCTAGCGAATTCTTTACATCCTCCGTTTCATGTT-3′ |
| | | R: 5′-AGCCATATGGCCATGGGGACACAAAAAGTCTTTAGCAC-3′ |
| | Q81UH1 | F: 5′-CTGGCCATGGAGGTTAGTAAAGTGTACACG-3′ |
| | | R: 5′-TAGCGGATCCCCTAACAAAAATCCCCG-3′ |
| Brucella abortus 9-941 | Q57B94 | F: 5′-CAGCCATATGGACATGTCAAGACATTCCTTCTTCTGC-3′ |
| | | R: 5′-TCATGCTAGCGAATTCGCC GCGCTGAACCC-3′ |
| Brucella melitensis 16M | Q8YJ29 | F: 5′-CAGCCATATGGACATGTCAAGACATTCCTTCTTCTGC-3′ |
| | | R: 5′-TCATGCTAGCGAATTCGCCGCGCTGAACCC-3′ |
| Brucella suis 1330 | Q8FYS0 | F: 5′-CAGCCATATGGACATGTCAAGACATTCCTTCTTCTGC-3′ |
| | | R: 5′-TCATGCTAGCGAATTCGCCGCGCTGAACCC-3′ |
| Burkholderia cenocepacia | AOAZQ0 | F: 5′-TCTGCTAGCGGATCCCGCACGAGAAAACCGTAG-3′ |
| HI2424 | | R: 5′-CAGCCATATGGCCATGGAAATTACCCGATCCCTTTC-3′ |
| | AOBOB8 | F: 5′-TCTGCTAGCGGATCCCGCACGAGAAAACCGTAG-3′ |
| | | R: 5′-CAGCCATATGGCCATGGAAATTACCCGATCCCTTTC-3′ |
| Burkholderia pseudomallei | Q63NG7 | F: 5′-TCATGCTAGCGGATCCGATGCGATGCCCCAGC-3′ |
| K96243 | | R: 5′-CAGCCATATGGCCATGGAGCACATCCACATCATCGATT-3′ |
| Clostridium difficile 630 | Q17ZY4 | F: 5′-TCATGCTAGCGGATCCTTAAGAATAAATCCATGTTTAAGTGG-3′ |
| | | R: 5′-TATGGACATGTTATTTAGCAGAAGTATACA-3′ |
| Pseudomonas aeruginosa | A91476 | F: 5′-TCATGCTAGCGGATCCCGGCGGATGCCCC-3′ |
| PAO1 | | R: 5′-CAGCCATATGGCCATGGAACGCATCCGCATCATC-3′ |
| | Q91489 | F: 5′-GGCCATGGGTTCGCAGCGGATCGTCC-3′ |
| | | R: 5′-CCAAGCTTGCAGTGGCCGCCGGGCCAG-3′ |
| Vibrio parahaemolyticus | A87Q20 | F: 5′-TCATGCTAGCGGATCCTTCACTTCAAATCCGTACGC-3′ |
| O3:K6 | | R: 5′-CAGCCATATGGCCATGGGACAAGGAACCTTTTTCTGTATC-3′ |

| | | |
|---|---|---|
| Pseudomonas aeruginosa | Q91476 | Forward and Reverse primers for Mutagenesis |
| PAO1 | | |

| | |
|---|---|
| C88S | F: 5′-GGTGCCGTGGCCGCTCATGCCGAGGTAGCC-3′ |
| | R: 5′-GGCTACCTCGGCATGAGCGGCCACGGCACC-3′ |
| C236S | F: 5′-TGGTGCCGGTGCCGCTGGGCGAGCGGTCGT-3′ |
| | R: 5′-ACGACCGCTCGCCCAGCGGCACCGGCACCA-3′ |
| V60G | F: 5′-GCAGCGACGTACTGGGCGGCGCCCTGCTC-3′ |
| | R: 5′-GAGCAGGGCGCCGCCCAGTACGTCGCTGC-3′ |
| V60F | F: 5′-GCAGCGACGTACTGTTCGGCGCCCTGCTCT-3′ |
| | R: 5′-AGAGCAGGGCGCCGAACAGTACGTCGCTGC-3′ |

Optimum racemization and epimerization conditions were determined using 20 mM L-Pro or OH-L-Pro in 0.2 M NaOAc or Tris 20 mM/EDTA 1 mM (TE) buffers, respectively, as a function of pH. Percent of racemization or epimerization of serial concentrations of substrate was calculated by incubating 3-10 μg of recombinant protein, 20-80 mM substrate in NaOAc pH 6 or TE, pH 8 (q.s.p. 500 μl) for 30-60 min at 37° C. The reactions were stopped by incubating at −20° C. and optical rotations measured in a polarimeter 241MC (Perkin Elmer) (1). Percent inhibition of enzymatic activities was determined incubating 10 μg of recombinant protein in presence or absence of 1-10 mM PYC, 1-25 mM iodoacetamide, or 1-25 mM iodoacetate. Control reactions were performed in presence or absence of PLP. All reagents were purchased from Sigma.

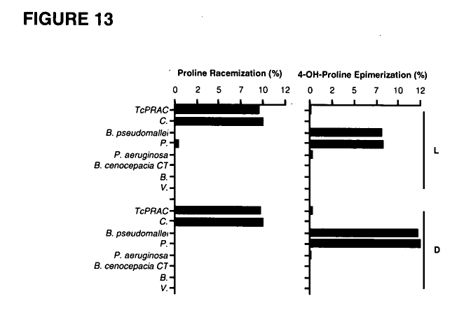

Purified recombinant proteins were analyzed in biochemical assays by measuring the shift in optical rotation of either L- or D-Pro. As shown in FIG. 13, C. difficile (Cd) recombinant protein racemizes both L- and D-Pro but not OH-UD-Pro or any other natural amino acid. CaPRAC activity is PLP-independent which closely resembles TcPRAC and CsPRAC (1, 27). Conversely, B. pseudomallei, P. aeruginosa, and three Brucella species recombinant proteins present no measurable PRAC activity but instead demonstrate strong epimerization of OH-UD-Pro behaving as genuine OH-Pro epimerases (HyPRE), as discussed below.

As predicted, control recombinant proteins produced from B. cenocepacia and P. aeruginosa sequences that present “Cys-Thr” or “Ser-Cys” coupled replacements, respectively, did not show either PRAC or HyPRE enzymatic activities. Unexpectedly, however, three tested recombinant proteins (two produced from Bacillus anthracis and one from Vibrio parahaemolyticus), annotated as “putative PRACs” and presenting the “Cys-Cys” couple, generated recombinant proteins that did not display PRAC or HyPRE activities.

These results emphasize that despite the increased availability of genome data, the attribution of putative functions to homologous gene annotations are at times too simple and errors can occur with the consequence of incorrect scientific dogmas. This invention reveals that from a selected database assembled from blast searches using T. cruzi proline racemase (Tc PRAC) full-length sequences, 73% of the hits were incorrectly annotated as PRAC or putative PRAC, since most of the proteins do not experimentally display functional PRAC activity.

Moreover, the present invention reveals that out of 12 “PRAC-like” recombinant proteins from different pathogens, only two (17%) from C. difficile and Tripanosoma vivax, demonstrate truthful PRAC activity. Other proteins isolated from bacteria responsible for human and animal health problems (refs 21-25 from manuscript) have been incorrectly annotated as PRAC and are in fact HyPRE, i.e. 3 Brucella species, P. aeruginosa and B. pseudomallei. In addition, 33% of the studied sequences were erroneously annotated despite missing the fundamental catalytic residues.

### EXAMPLE 21

### Brucella abortus PrpA Virulence Factor is a Validated Hydroxyproline Epimerase

One of the B. abortus sequences, presenting the “Cys-Cys” couple, was reported elsewhere as a B-cell mitogen with PRAC activity (BaPrpA, for proline racemase protein A) and was shown to be directly involved in bacterial virulence and immune system evasion (28). Surprisingly, those authors described PrpA as displaying discrete racemization of L-Pro but as being unable of catalyzing the reverse reaction (i.e., conversion of D-Pro enantiomers). If correct, that assertion would imply that other PRACs would behave likewise. To address this, the enzymatic activity of BaPrpA produced from SEQ ID NO: 137 obtained in silico was investigated. BaSeq1, derived from Ba-strain 544, is 100% homologous to Ba-strain 9-941 and BaPrpA (Ba strain 2308, BAB1_{—}1800) and possesses all PRAC motifs (FIG. 20).

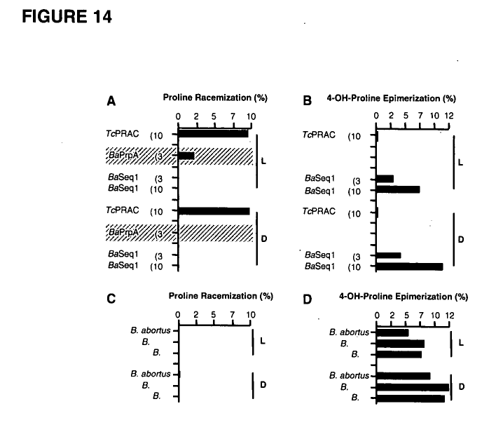

The present invention undoubtedly demonstrates that BaSeq1 displays only HyPRE activity irrespective of enzyme concentration, pH, and buffer conditions (FIGS. 14A and 14B), in contrast to recurrent Pro racemization values obtained with TcPRAC. Since the BaSeq1 sequence is 98% homologous to proteins from B. melitensis (Bm) and B. suis (Bs), all three recombinant homologous proteins were tested in parallel for PRAC and HyPRE activities. Those proteins are unable to catalyze Pro racemization but exhibit equivalent strong ability to perform epimerization of both OH-L-Pro and OH-D-Pro (FIGS. 14C and 14D). Consequently, the data proves that BaSeq1, and therefore PrpA, is a HyPRE, as are the B. melitensis and B. suis corresponding proteins. Accordingly, this invention provides functional HyPREs from P. aeruginosa and several other important pathogens such as B. pseudomallei and Brucella spp., which are agents of melioidosis and brucellosis, respectively (29, 30, 31).

This invention provides a clarification of earlier work (28) concerning an immunomodulatory virulence factor (PrpA) of B. abortus, that possibly due to its 40% homology with TcPRAC, was described as a PRAC. Surprisingly, PrpA was described as displaying discrete racemization of L-Pro but as being unable to catalyze the conversion of D-Pro enantiomer. As such, that data would imply that some racemases do not follow fundamental racemic principles. In contrast, the present data establishes that PrpA from B. abortus, B. melitensis and B. suis are in fact HyPRE that catalyze the interconversion of OH-UD-Pro. These results are significant to prevent any misinterpretations of mechanisms linked to pathogenesis induced by Brucella spp.

### EXAMPLE 22

### Kinetic Properties of Bacterial Hydroxyproline Epimerases

Kinetic assays were performed at 37° C. with 10-160 mM of each substrate, 20 μg/ml of specific enzymes in optimum reaction buffer (2). After determination of the linear part of the curve, velocity in 10-160 mM substrate was measured every 30 sec during 5 min to determine Kₘ and Vₘₐₓ.

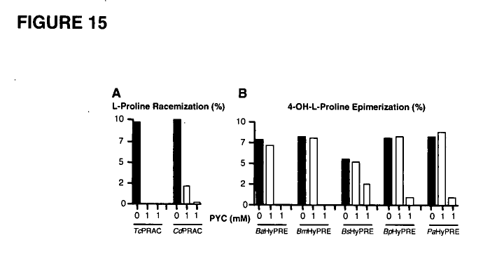

Optimum conditions for PRAC and HyPRE reactions for all bacterial enzymes were obtained in NaOAc, pH 6 and Tris/EDTA (TE), pH 8-9 buffers, respectively. On the other hand, while PRAC was radically inhibited by its specific competitive inhibitor pyrrole-2-carboxylic acid (PYC), no inhibition of HyPRE was observed with standard amounts of PYC (1 mM) (FIGS. 15A and 15B). HyPRE reactions were only affected by high amounts of PYC (10 mM) or by variable concentrations of iodoacetate and iodoacetamide inhibitors (FIG. 21).

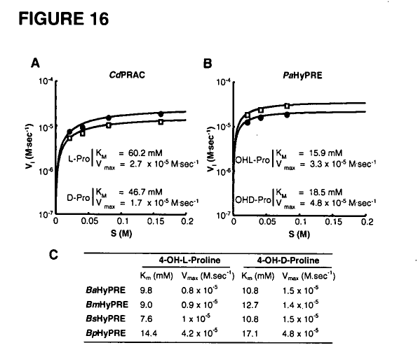

Progress of Pro and OH-Pro catalysis was monitored polarimetrically. The interconversion of L to D-Pro mediated by CdPRAC revealed that the enzyme has comparable velocity and affinity constants to those of TcPRAC (FIG. 16). Graphic representation of the Michaelis-Menten equation corresponding to the initial velocity of CdPRAC and PaHyPRE as a function of substrate concentration is shown, as well as respective Kₘ and Vₘₐₓ kinetic values (FIGS. 16A and 16B). Brucella spp and B. peudomallei HyPREs exhibited comparable Vₘₐₓ and apparent Kₘ values to those of PaHyPRE (FIG. 4C). However, at equilibrium, all HyPRE enzymes showed a clear advantage to OH-D-Pro substrate.

### EXAMPLE 23

### Additional Elements to Previously Defined PRAC Signature can Discriminate Proline Racemases and Hydroxyproline Epimerases

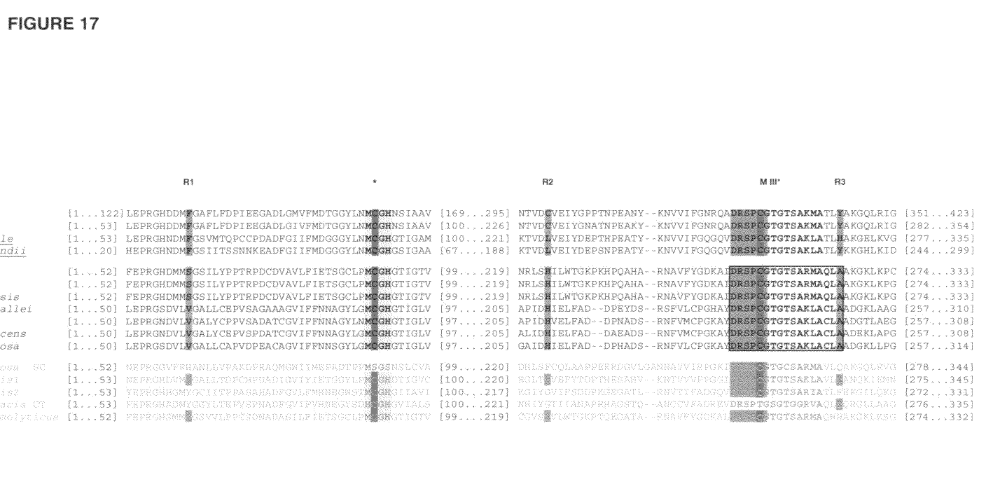

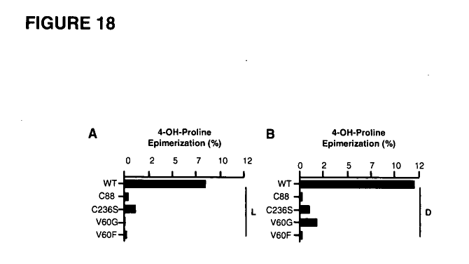

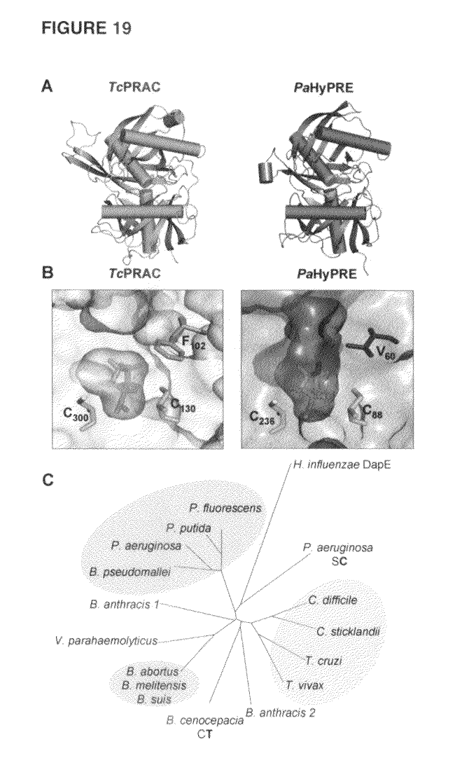

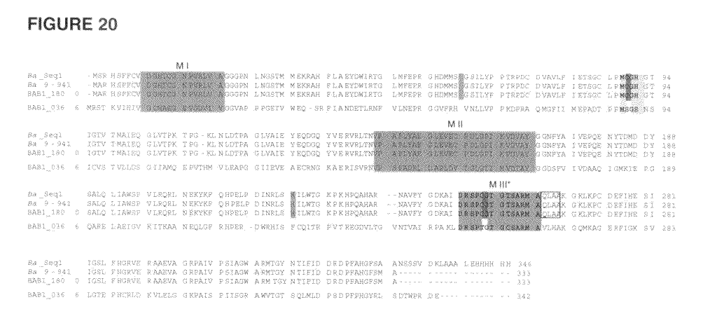

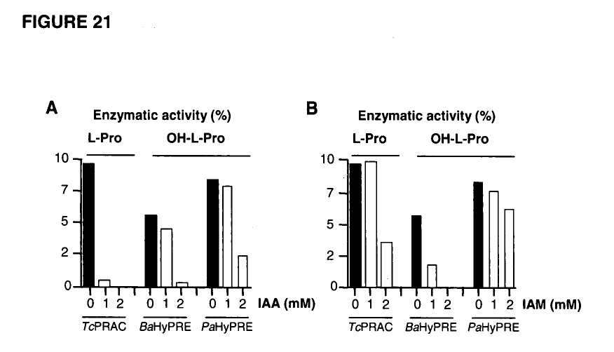

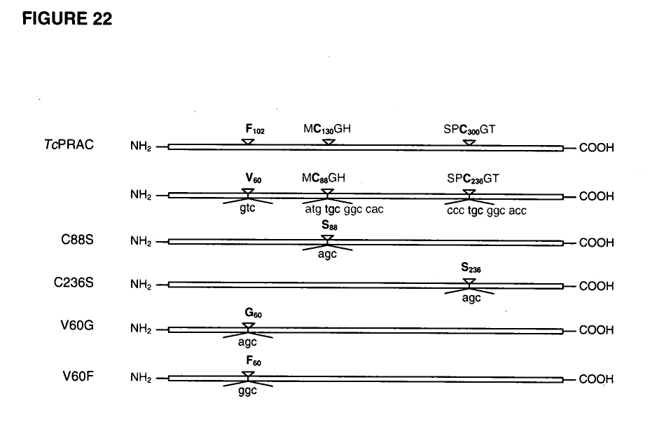

As discussed above, the Blast searches using full-length TcPRAC sequence, MIII* and MCGH block, revealed that a number of homologous hits actually corresponded to HyPRE, a PRAC-related enzyme. Sequences of PRAC and HyPRE were aligned and residues that may be useful for their discrimination were identified (FIG. 17). Although both enzymes possess the catalytic “Cys-Cys” couple, three major and non dissociated differences seem to be noteworthy for substrate specificity.

The first and most important particularity is an aromatic Phe residue, which was shown to be capital to hydrophobic contacts of TcPRAC with Pro ring carbon atoms, that is missing in HyPRE (depicted in R1). In fact, Phe imposes polarity constraints precluding polar functions at the level of the substrate carbon ring. In contrast, HyPRE holds Ser or Val substitutions, i.e. small polar or aliphatic amino acids, that would account for better OH-Pro accessibility into the pocket. Other sequences encoding proteins without enzymatic activity may present at that position, e.g., polar Tyr or His residues which would restrict PRAC or HyPRE catalysis, as observed with B. anthracis sequences.

Another feature identified by this invention is the presence in the TcPRAC pocket environment of a Cys (or a Leu, for other PRAC) residue in position 270 while HyPREs possess in that position a consistent polar His residue (depicted in R2) optimally placed to favor H-bonding interaction with the OH— of the C^{γ}-atom of OH-Pro. Finally, an additional block of three residues downstream of the highly conserved MIII* (XLA, depicted in R3) was identified as being fully restrictive to discriminate HyPRE and PRAC enzymes. These three differences are complementary to the presence of the “Cys-Cys” couple of the catalytic pockets as ascertained by the absence of both enzymatic activities exhibited by B. anthracis and V. parahaemolyticus proteins.

Accordingly, based on overall comparisons between PRAC and HyPRE and despite the evident identities displayed by the peptide sequences, this invention provides structural evidences that allow the discrimination of both enzymatic activities. PRAC and HyPRE multiple alignments allowed identification of other important and non dissociated elements that account for the differentiation of the enzymes, such as the presence of the aliphatic Cys (or Leu) residue in TcPRAC at position 270, which is absent and replaced by a polar His residue in HyPRE, thus favouring its interaction with OH-Pro. Additionally, a block of residues (XLA) downstream of the previously identified minimal MIII* PRAC signature (3) was found to be HyPRE-specific. The combination of those elements are fundamental in shaping the binding pocket and thus determining the substrate specificity as supported by the detailed structural analysis of TcPRAC and PaHyPRE active sites. Apart from previous work using purified P. putida HyPRE which associated the enzyme active site to 14 residues (32), the current work is the first describing HyPRE full-length genes and may contribute in the future to better annotation of unknown ORFs.

### EXAMPLE 24

### Abrogation of HyPRE Enzymatic Activity by Mutation of Conserved Cysteine Residues of the Catalytic Site

Site-directed mutagenesis of PaHyPRE was performed using a QuikChange® XL kit (Stratagene), as described (25), to obtain the point mutants C88S, C236S, V60F, and V60G. Briefly, point mutations were obtained by PCR using forward and reverse overlapping mutagenic primers (FIG. 22 and Table XIV). Plasmids were purified and point mutations were ascertained by sequencing. Recombinant proteins were produced from each point mutant, as described above.

The HyPRE homodimer was described as having both subunits participating in a single catalytic site (32, 33). The potential role of the “Cys-Cys” couple in HyPRE catalysis was verified through site-directed mutagenesis of PaHyPRE Cys₈₈ or Cys₂₃₆ into Ser residues (Table XIV and FIG. 22). In comparison to wild type HyPRE, ^{C88S}HyPRE and ^{C236S}HypRE single mutations induced radical loss of OH-UD-Pro epimerization establishing that proton transfer during HyPRE reaction is indeed dependent on the presence of the catalytic “Cys-Cys” couple of each subunit (FIG. 18).

To validate the weight of the Val₆₀ residue in ligand accessibility and thus in substrate specificity, the residue was mutated into Gly (^{V60G}HyPRE) or Phe (^{V60F}HyPRE), meeting or not size and stability limits imposed by Val. The absence of epimerization exhibited by the two mutants reveals that the Val₆₀ aliphatic residue indeed accounts for OH-Pro ligand specificity and is consequently essential for HyPRE catalysis. Conversely, the Phe₁₀₂ residue on the PRAC catalytic site environment offers hydrophobic restriction area to the pocket occupancy restraining the accessibility of OH-Pro.

The space and polarity constraints of PRAC and HyPRE active sites on protein-ligand interactions were visualized better by comparing the closer views of the enzyme pockets (FIGS. 19A and 19B). Therefore, despite close similarities displayed by PRAC and HyPRE 3D-structures, the presence of a sizable aromatic residue or, alternatively, of a small aliphatic or polar amino acid, unquestionably plays a determinant role on the enzyme/substrate specificity.

Considering that the results obtained with PaHyPRE mutants support the key role of Cys₈₈ and Cys₂₃₆ residues in catalysis and the large overall structural similarity with TcPRAC, this invention supports a reaction mechanism similar to PRAC where HyPRE equally possesses two active sites per dimer, each one including two catalytic Cys. However, HyPRE is not inhibited by PYC, the transition state analogue of Pro. It has previously been shown that hydrophobic Phe₁₀₂ and Phe₂₉₀ residues present in the TcPRAC pocket impose polarity restrictions that enable interactions of the enzyme with the C^{α} of proline ring or the C2 atom of PYC. Instead, the absence of Phe residues in HyPRE pocket, most particularly Phe₁₀₂, and its substitution by an aliphatic Val (or polar Ser), promotes an ideal environment for accessibility and stereoinversion of the C^{α} of OH-Pro. Indeed, mutagenesis of Val₆₀ into Gly or Phe, results in radical loss of PaHyPRE activity, attributing a significant role to Val₆₀ in the conformation of the enzyme, the pocket stability and the ligand specificity.

The significance and conservation of PRAC and HyPRE throughout evolution was investigated by a phylogram using another PLP-independent enzyme as an uncontroversial outgroup, i.e. the Haemophilus influenzae diaminopimelate epimerase (DapE). FIG. 19C shows that PRAC and HyPRE cluster in three main groups. Interestingly, since PRAC from C. difficile and C. sticklandii cluster together with T. cruzi and T. vivax, it is conceivable that the divergence of the tree branches reflects their ancient origin and thus are phylogenetically older than the separation of bacteria, archea and eukaryotes. Alternatively, possible gene transfer between species can be envisaged.

### EXAMPLE 25

### Lymphocyte Proliferation Assays

PRAC enzymes, as other B-cell mitogens, have been described as being involved in evasion mechanisms of parasite and bacterial species through the induction of non-specific hypergammaglobulinemia and by the secretion of pleiotropic cytokines (1, 34). Accordingly, lymphocyte proliferation assays were carried out in presence of 2×10⁵ splenocytes of Balb/c mice (male 9 week-old, Janvier, Le Genest-St-Isle, France) per well in 96-well plates in presence or absence of recombinant T. cruzi or C. difficile PRACs, P. aeruginosa or B. abortus HyPREs.

Assays were compared to spleen cell proliferation obtained with cells cultivated with medium alone or cells stimulated with lipopolysaccharide from E. coli O5:B55 (LPS, 5 μg/ml final) and Concanavalin A (Con A 2.5 μg/ml final)—unrelated B and T cell mitogens, as controls. All cultures were performed in presence of polymixine-B (PMB, 2 μg/ml final). Final concentration of endotoxin found in the samples before addition of polymixine-B was as follows: LPS (5000 ng/ml); TcPRAC (0.1 ng/ml); CdPRAC (20 ng/ml), PaHyPRE (30 ng/ml); BaHyPRE (40 ng/ml). Cultures were kept at 37° C. in 5% CO₂ and pulsed with 1 μCi ^{[3H]}Thymidine per well for 17 h before harvesting. C.P.M. were determined using a β-counter 1450 Microbeta Trilux (Perkin-Elmer) at 24, 48, 72 and 96 h.

The results of these experiments are presented in Table XV and are expressed as Stimulation Index obtained by dividing the average c.p.m. from stimulated cells by the average c.p.m from unstimulated cells. The data reveal that all PRAC and HyPRE proteins induce significantly high levels of spleen cell proliferation that are independent of any contamination with endogenous endotoxin, as compared to the ^{[3H]}Thymidine uptake of cultures stimulated with LPS, a classical polyclonal B-cell activator. Note that PMB concentration used in the cultures was sufficient to induce 50% reduction of LPS-triggered B cell activation and proliferation.

Accordingly, this invention demonstrates that similarly to TcPRAC, PRAC from C. difficile, HyPRE from P. aeruginosa and HyPRE from B. abortus are also strong lymphocyte mitogens, as they increase in vitro lymphoproliferation by up to 10 fold. It has been shown that mitogen-induced proliferation of resting lymphocytes is associated with a marked increase in amino acid uptake and intracellular enzyme pathways to meet the demands of increased cellular protein synthesis (35). It is relevant that enzymes of Pro biosynthesis, and not those of Pro degradation, are particularly increased with lymphocyte activation. However, with sufficient amounts of exogenous Pro, large increases are observed of pyrroline-5-carboxylate reductase (PCA reductase), a key enzyme in Pro synthesis. Isoforms of PCA reductase, sensitive and insensitive to feedback inhibition by Pro do exist (36). Interestingly, PCA reductase from distinct tissues differs according to its sensitivity to Pro-inhibition. Considering tissue specificity and tropism of infectious pathogens, it would not be surprising that upon infection PRAC and HyPRE play important roles in the regulation of the intracellular and extracellular amino acid pool to take advantage of some host precursors and enzymatic pathways.

TABLE XV
Mitogenic activity of PRAC and HypRE enzymes.
Stimulation index

Time

| | 24 h | 48 h | 72 h | 96 h |
|---|---|---|---|---|
| LPS | 24.0 | 120.4 | 71.2 | 26.3 |
| LPS + PMB (50%) | 12.0 | 47.1 | 32.0 | 12.9 |
| Recombinant proteins | | | | |
| TcPRAC + PMB | 0.8 | 2.7 | 5.6 | 7.9 |
| CdPRAC + PMB | 3.1 | 13.0 | 14.7 | 8.9 |
| PaHyPRE + PMB | 2.6 | 13.6 | 12.8 | 6.7 |
| BaHyPRE + PMB | 3.1 | 12.8 | 15.1 | 9.1 |

In summary, this invention presents the identification and characterization of two novel proline racemases from Clostridium difficile and Tripanosoma vivax and of five novel hydroxyproline-2-epimerases from Pseudomonas aeruginosa, Burkholderia pseudomallei, Brucella melitensis, Brucella suis, and Brucella abortus. This invention also provides sequence elements for discriminating proline racemases from hydroxyproline epimerases, namely Phe¹⁰² (R1), Cys²⁷⁰ (R2), and XLA immediately downstream of Motif III* (depicted in R3). In addition, this invention identifies three critical residues for the enzymatic function of HyPREs, namely Cys⁸⁸, Cys²³⁶, and Val⁶⁰. This invention also involves the use of PRAC or HyPRE antigens, antibodies, or mitogens as vaccinating agents without inducing lymphocyte polyclonal activation. Thus, this invention not only prevents infection by the pathogen, but also avoids the negative consequences of such infection (immunosuppression, persistent infection, and susceptibility to immunopathology and autoimmune phenomenon).

The following E. coli strains were deposited at the Collection Nationale de Cultures de Microorganismes (C.N.C.M.), of Institut Pasteur, 25, rue du Docteur Roux, F-75724 Paris, Cedex 15, France:

| Identification Reference | GenBank Number | Accession Number |
|---|---|---|
| BmHyPRE | EF495342 | CNCM I-3813 |
| BpHyPRE | EF495345 | CNCM I-3816 |
| PaHyPRE | EF495341 | CNCM I-3815 |
| BsHyPRE | EF495343 | CNCM I-3814 |
| CdPRAC | EF495346 | CNCM I-3817 |
| BaHyPRE | EF495344 | CNCM I-3812 |

### REFERENCES

The following references are incorporated by reference, in their entirety, herein.

¹ GenBank accession number AF195522
² GenBank accession number AY140947
³ EMBL accession number E10199.

### Bibliography Related to the Microtest Using D-Amino Acid Oxidase According to the Invention:

1. Reina-San-Martin B., Degrave W., Rougeot C., Cosson A., Chamond N., Cordeiro-da-Silva A., Arala-Chaves M., and Minoprio P. (2000) A B-cell mitogen from a pathogenic trypanosome is a eukaryotic proline racemase. Nature Medicine, 6, 890.
2. Chamond N., Gregoire C., Coatnoan N., Rougeot C., Freitas-Junior L. H., da Silveira J. F., Degrave W. M., and Minoprio P. (2003) Biochemical characterization of proline racemases from the human protozoan parasite Trypanosoma cruzi and definition of putative protein signatures. J Biol Chem, 278, 15484.
3. Chamond N., Coatnoan N., and Minoprio P. (2002) Immunotherapy of Trypanosoma cruzi infections. Current Drug Targets, 2, 247.
4. Rassi A. and Luquetti A. O. (1992) Therapy of Chagas Disease. In: Chagas Disease (American Trypanosomiasis): its impact on transfusion and clinical medicine (ed. S. Wendel, Z. Brener, M. E. Camargo & A. Rassi), p. 237. ISBT Brazil' 92—SBHH, Sao Paulo.
5. Cancado J. R. (1999) Criteria of Chagas disease cure. Mem Inst Oswaldo Cruz, 94 Suppl 1, 331.
6. Urbina J. A. (2001) Specific treatment of Chagas disease: current status and new developments. Curr Opin Infect Dis, 14, 733.
7. Urbina J. A. (2002) Chemotherapy of Chagas disease. Curr Pharm Des, 8, 287.
8. Donald G. and McNeil Jr. (2003) Rare Infection Threatens to Spread in Blood Supply, in New York Times, Nov. 18, 2003
9. Beard C., Pye G. Steurer F. J., Rodriguez R., Campman R., Townsend Peterson A., Ramsey J., Wirtz R. A. and Robinson, L. E. Chagas Disease in a Domestic Transmission Cycle, Southern Texas, USA. (2003) Emerging Infectious Diseases, 9, 103.
10. Hamase K., Morikawa A. and Zaitsu K. (2002) D-amino acids in mammals and their diagnostic value. J Chromatogr B Analyt Technol Biomed Life Sci, 781, 73.
11. D'Aniello A., Lee J. M., Petrucelli L. and Di Fiore M. M. (1998) Regional decreases of free D-aspartate levels in Alzheimer's disease. Neurosci Lett, 250, 131.
12. D'Aniello A., Di Fiore M. M., Fisher G. H., Milone A., Seleni A., D'Aniello S., Perna A. F. and Ingrosso D. (2000) Occurence of D-aspartic acid and N-methyl-D-aspartic acid in rat neuroendocrine tissues and their role in the modulation of luteinizing hormone and growth hormone release. FASEB J, 14, 699.
13. Fisher G. H., D'Aniello A., Vetere A., Padula L., Cusano G. P. and Man E. H. (1991) Free D-aspartate and D-alanine in normal and Alzheimer brain. Brain Res Bull, 26, 983.
14. Fisher G. H., Torres D., Bruna J., Cerwinski S., Martin T., Bergljung C., Gruneiro A., Chou S. J., Man E. H. and Pappatheodorou S. (1995) Presence of D-aspartate and D-glutamate in tumor proteins. Cancer Biochem Biophys, 15, 79.
15. Fisher G., Lorenzo N., Abe H., Fujita E., Frey W. H., Emory C., Di Fiore M. M. and A D. A. (1998) Free D- and L-amino acids in ventricular cerebrospinal fluid from Alzheimer and normal subjects. Amino Acids, 15, 263.
16. Fisher G. H. (1998) Appearance of D-amino acids during aging: D-amino acids in tumor proteins. Exs, 85, 109.
17. Nagata Y., Akino T., Ohno K., Kataoka Y., Ueda T., Sakurai T., Shiroshita K. and Yasuda T. (1987) Free D-amino acids in human plasma in relation to senescence and renal diseases. Clin Sci (Colch), 73, 105.
18. Nagata Y., Masui R. and Akino T. (1992) The presence of free D-serine, D-alanine and D-proline in human plasma. Experientia, 48, 986.
19. Chouinard M. L., Gaitan D. and Wood P. L. (1993) Presence of the N-methyl-D-aspartate-associated glycine receptor agonist, D-serine, in human temporal cortex: comparison of normal, Parkinson, and Alzheimer tissues. J Neurochem, 61, 1561.
20. Kumashiro S., Hashimoto A. and Nishikawa T. (1995) Free D-serine in post-mortem brains and spinal cords of individuals with and without neuropsychiatric diseases. Brain Res, 681, 117.
21. Wellner D. and L. A. Lichtenberg, (1968), Assay of Amino acid oxidase, Methods in Enzymology XVII, “metabolism of Amino acids”, 593
22. Scannone H., D. Wellner and A. Novogrodsky, (1964), A study of amino acid oxidase specificity, using a new sensitive assay, Biochemistry, 3, 1742.
23. Kishimoto M. and Takahashi T., (2001), A spectrophotometric microplate Assay for L-amino acid oxidase, Analytical Biochemistry, 298, 136.
24. Wolosker H., Sheth K. N., Takahashi M., Mothet J.-P., Brady R. O. Jr, Ferris, C. D. and Snyder S. H., (1999), Purification of serine racemase: Biosynthesis of the neuromodulator D-Serine, Proc. Nat. Acad. Sci. USA, 96, 721.
25. Buschiazzo A., Goytia M., Schaeffer F., Degrave W., Shepard W., Gregoire C., Chamond N., Cosson A., Berneman A., Coatnoan N., et al. (2006) Proc Natl Acad Sci USA 103, 1705-1710.
26. Chamond N., Goytia M., Coatnoan N., Barale J. C., Cosson A., Degrave W. M., and Minoprio P. (2005)Mol Microbiol 58, 46-60.
27. Cardinale G. J. and Abeles R. H. (1968) Biochemistry 7, 3970-3978.
28. Spera J. M., Ugalde J. E., Mucci J., Comerci D. J. and Ugalde R. A. (2006) Proc Natl Acad Sci USA 103, 16514-16519.
29. Godfroid J., Cloeckaert A., Liautard J. P., Kohler S., Fretin D., Walravens K., Garin-Bastuji B., and Letesson J. J. (2005) Vet Res 36, 313-326.
30. Pappas G., Akritidis N., Bosilkovski M., and Tsianos E. (2005) N Engl J Med 352, 2325-2336.
31. Wiersing a W. J., van der Poll T., White N. J., Day N. P., and Peacock S. J. (2006) Nat Rev Microbiol 4, 272-282.
32. Ramaswamy S. G. (1984) J Biol Chem 259, 249-254.
33. Adams E. and Norton I. L. (1964) J Biol Chem 239, 1525-1535.
34. Reina-San-Martin B., Cosson A. and Minoprio P. (2000) Parasitol Today 16, 62-67.
35. Valle D., Blaese R. M. and Phang J. M. (1975) Nature 253, 214-216.
36. Valle D. Downing S. J. and Phang J. M. (1973) Biochem Biophys Res Comm 54, 1418-1424.
37. Gryder R. M. and Adams E. (1969) J Bacteriol 97, 292-306.
38. Miyoshi S, and Shinoda S. (2000) Microbes Infect 2, 91-98.
39. de Bentzmann S., Polette M., Zahm J. M., Hinnrasky J., Kileztky C., Bajolet O., Klossek J. M., Filloux A., Lazdunski A. and Puchelle E. (2000) Lab Invest 80, 209-219.
40. Harrington D. J. (1996) Infect Immun 64, 1885-1891.
41. Bejarano P. A., Langeveld J. P. I., Hudson B. G. and Noelken M. E. (1989) Infect Immun 57, 3783-3787.
42. Lamzin V. S., Dauter Z., and Wilson K. S. (1995) Curr Opin Struct Biol 5, 830-836.
43. Kleinkauf H., and von Dohren H. (1987) Annu. Rev. Microbiol. 41, 259-289.
44. Nagata Y., Fujiwara T., Kawaguchi-Nagata K., Fukumori Y., and Yamanaka T. (1998) Biochim Biophys Acta 1379, 76-82.
45. Nagata Y., Tanaka K., Iida T., Kera Y., Yamada R., Nakjima Y., Fujiwara T., Fukumori Y., Yamanaka T., Koga Y., Tsuji S., and Kawaguchi-Nagata K. (1999) Biochim Biophys Acta 1435, 160-166.
46. Oguri S., Kumazaki M., Kitou R., Nonoyama H., and Tooda N. (1999) Biochim Biophys Acta 1472, 107-114.
47. Neidle A. and Dunlop D. S. (1990) Life sci. 46, 1512-1522.
48. Schell M. J., Molliver M. E., and Snyder S. H. (1995) Proc. Nat. Acad. Sci. 92, 3948-3952.
49. Wolosker H., Blackshaw S., and Snyder S. H. (1999) Proc Natl Acad Sci USA 96, 13409-13414.
50. Nagata Y., Homma H., Matsumoto M., and Imai K. (1999) FEBS 454, 317-320. Rudnick G. and Abeles R. H. (1975) Biochemistry 14, 4515-4522.
51. Cano M. I., Gruber A., Vazquez M. Cortes A., Levin M. J., Gonzalez A., Degrave W., Rondinelli E., Zingales B., Ramirez J. L., Alonso C., Requena J. M., and Silveira J. F. D. (1995) Mol. Bio. Par. 71, 273-278.
52. Higuchi R., Krummel B., and Saiki K. K. (1988) Nuc. Ac. Res. 16, 7351-7367.
53. Keenan M. V. and Alworth W. L. (1974) Biochem Biophys Res Commun 57, 500-504.
54. Fisher L. M., Albery W. J., and Knowles J. R. (1986) Biochemistry 25, 2529-2537.
55. Albery W. J. and Knowled J. R. (1986) Biochemistry 25, 2572-2577.
56. Breitbart R. E., Andreadis A., and Nadal-Ginard B. (1987) Annu. Rev. Biochem. 56, 467-495.
57. Manning-Cela R., Gonzalez A., and Swindle J. (2002) Infect. Immun. 70, 4726-4728.
58. Krassner S. M. and Flory B. (1972) J. Protozool. 19, 917-920.
59. Bowman I. B. R., Srivastava H. K., and Flynn I. W. (1972) Adaptatoin in oxidtive metabolism during the transformation of Trypanosoma rhodesiense from bloodstream into culture forms, Van den Bossche H. Ed. Comparative Biochemistry of Parasites, Academic Press, New York.
60. Evans D. a. and Brown R. C. (1972) J. Protozool. 19, 686-690.
61. Auerswald L., Schneider P., and Gade G. (1998) J. Exp. Biol. 201, 2333-2342.
62. Sylvester D. and Krassner S. M. (1976) Comp. Biochem. Physiol. 55B, 443-447.
63. de Isola E. L., Lammel E. M., Katzin V. J., and Gonzalez Cappa, S. M. (1981) J. Parasitol. 67, 53-58.
64. Contreras V. T., Salles J. M., Thomas N., Morel C. M., and Goldenberg S. (1985) Mol. Biochem. Parasitol. 16, 315-327.
65. Silber A. M., Tonelli R. R., Martinelli M., Colli W., and ALves M. J. (2002) J. Eukaryot. Microbiol. 49, 441-446.
66. Janeway C. A. and Humphrey J. H. (1970) Folia Biol. 16, 156-172.
67. Mozes E., Kohn L. D., Hakim F., and Singer D. S. (1993) Science 261, 91-92.
68. Sela M. and Zisman E. (1997) FASEB J. 11, 449-456.
69. Contreras V. T., Morel C. M., and Goldenbarg S. (1985) Mol Biochem Parasitol 14, 83-96.
70. Souto-Padron T., Reyes M. B., Leguizamon S., Campetella O. E., Frash A. C., and de Souza W. (1989) Eur. j. Cell Biol. 50, 272-278.
71. Janes B. K. and Bender R. A. (1998) J Bacteriol 180, 563-570.
72. de Jong M. H., van der Drift C., and Vogels G. D. (1975) J. Bacteriol. 123, 824-827.
73. Shakibaei M. and Frevert U. (1996) J. Exp. Med. 184, 1699-1711.
74. Burleigh B. A. and Andrews N. W. (1998) Curr. Op. Microbiol. 1, 461-465.
75. Gao W. Wortis H. H., and Pereira M. A. (2002) Internat. Immunol 14, 299-308.
76. Martin D., Ault B., and Nadler J. V. (1992) Eur. J. Pharmacol 216, 59-66.
77. Van Harreveld A. (1980) J. Neurobiol. 11, 519-529.
78. Thompson R. J., Bouwer H. G., Portnoy D. A., and Frankel F. R. (1998) Infect Immun 66, 3552-3561.
79. Watanabe T., Shibata K., Kera Y., and Yamada R. (1998) Amino Acids 14, 353-360.
80. Stadtman T. C. and Elliott P. (1957) J Biol Chem 228, 983-997.
81. Belasco J. G., Albery W. J. and Knowles J. R. (1986) Biochemistry 25, 2552-2558.
82. Yoshimura T. and Esak N. (2003) J Biosci Bioeng 96, 106-109.
83. Adams E. (1959) J Biol Chem 234, 2073-2084.
84. Radhakrishnan A. N. and Meister A. (1957) J Biol Chem 226, 559-571.
85. Adams E. (1970) Int Rev Connect Tissue Res 5, 1-91.
86. Prokop D. J. and Kivirikko K. I. (1995) Annu Rev Biochem 64, 403-434.
87. Bouza E., Munoz P., and Alonso R. (2005) Clin Microbiol Infect 11 Suppl 4, 57-64.
88. Stoddart B., and Wilcox M. H. (2002) Curr Opin Infect Dis 15, 513-518.
89. Kipnis E., Sawa T., and Weiner-Kronish J. (2006) Med Mal Infect 36, 78-91.
90. Sadikot R. T., Blackwell T. S., Christman J. W., and Prince A. S. (2005) Am J Respir Crit. Care Med 171, 1209-1223.
91. Macfarlane L., Oppenheim B. A., and Lorrigan P. (1991) J Infect 23, 346-347.
92. Gryder R. M. & Adams E. (1970) J Bacteriol 101, 948-958.

1-54. (canceled)

55. A method of identifying a target amino acid sequence as a putative proline racemase, wherein the method comprises:
(A) providing an amino acid sequence database;
(B) performing a computer assisted search of the database to compare a known amino acid sequence of a proline racemase with sequences in the database;
(C) identifying a target amino acid sequence in the database having at least about 25% homology to the known proline racemase amino acid sequence;
wherein the target sequence is a putative proline racemase if:
(1) the target sequence has the motif MIII* [SEQ ID NO: 4];
(2) the target sequence has the motif MCGH;
(3) the target sequence has two catalytic Cys residues corresponding to Cys₁₆₀ and Cys₃₃₀ (or Cys₁₃₀ and Cys₃₀₀ when the signal sequence is not present) of Trypanosoma cruzi racemase (TcPRAC) [SEQ ID NO: 30]; and
(4) the target sequence has a phenylalanine residue corresponding to Phe₁₃₂ (or Phe₁₀₂ when the signal sequence is not present)(R1) of TcPRAC; and
(5) the target sequence has a Cys or Leu (R2) residue corresponding to Cys₃₀₀ (or Cys₂₇₀ when the signal sequence is not present) of TcPRAC.

56. The method as claimed in claim 55, wherein the known proline racemase is TcPRAC [SEQ ID NO: 30], EF495346 (CdPRAC, C. difficile VPI10463) [SEQ ID NO: 141], or EF175213 (TVPRAC, T. vivax) [SEQ ID NO:143].

57. A method for the catalyzed conversion of one enantiomer to another enantiomer, wherein the method comprises:
(A) providing a proline racemase selected from EF495346 (CdPRAC, C. difficile VPI10463) [SEQ ID NO: 141] and EF175213 (TvPRAC, T. vivax) [SEQ ID NO:143];
(B) reacting the proline racemase with a substrate for the racemase in the presence of a buffer and at a pH for stereoinversion of chiral centers in the substrate to thereby form one or more of the enantiomers.

58. A method of identifying a target amino acid sequence as a putative epimerase, wherein the method comprises:
(A) providing an amino acid sequence database;
(B) performing a computer assisted search of the database to compare a known amino acid sequence of a proline racemase with sequences in the database;
(C) identifying a target amino acid sequence in the database having at least about 25% homology to the known proline racemase amino acid sequence;
wherein the target sequence is a putative epimerase if:
(1) the target sequence has the motif MIII* [SEQ ID NO: 4];
(2) the target sequence has the motif MCGH;
(3) the target sequence has two catalytic Cys residues corresponding to Cys₁₆₀ and Cys₃₃₀ (or Cys₁₃₀ and Cys₃₀₀ if the signal sequence is not present) of Trypanosoma cruzi racemase (TcPRAC) [SEQ ID NO: 30]; and
(4) the target sequence has a Ser or Val residue corresponding to Phe₁₃₂ (or Phe₁₀₂ when the signal sequence is not present) (R1) of TcPRAC; and
(5) the target sequence has a H is residue corresponding to Cys300 (or Cys₂₇₀ when the signal sequence is not present) of TcPRAC; and
(6) the target sequence has the R3 motif [SEQ ID NO: 130], which is absent from TcPRAC.

59. The method as claimed in claim 58, wherein the known proline racemase is TcPRAC [SEQ ID NO: 30], EF495346 (CdPRAC, C. difficile VPI10463) [SEQ ID NO: 141], or EF175213 (TvPRAC, T. vivax) [SEQ ID NO:143].

60. A method for the catalyzed epimerization of OH-L-Pro and OH-D-Pro of 4-hydroxyproline, wherein the method comprises:
(A) providing at least one epimerase selected from:
EF495341 (PaHyPRE, P. aeruginosa PAK) [SEQ ID NO: 131],
EF495342 (BmHyPRE, B. melitensis 16M) [SEQ ID NO: 133],
EF495343 (BsHyPRE, B. suis 1330) [SEQ ID NO: 135],
EF495344 (BaHyPRE, B. abortus 544) [SEQ ID NO 137], and
EF495345 (BpHyPRE, B. pseudomallei K96243) [SEQ ID NO: 139]; and
(B) reacting the epimerase with 4-hydroxyproline in the presence of a buffer and at a pH for OH-L/D-Pro epimerization.

61. A method of reducing catalytic activity of an epimerase, wherein the method comprises modifying at least one amino acid of the epimerase.

62. The method of claim 61 wherein the method comprises:
(A) modifying the epimerase by mutagenesis of at least one Cys residue corresponding to Cys₈₈ and Cys₂₃₆ of PaHyPRE; or
(B) modifying the epimerase by mutagenesis of a Val or a Ser residue of the epimerase corresponding to Phe₁₀₂ of TcPRAC; or
(C) both (A) and (B).

63. A method of detecting a substrate for a biologically active protein, wherein the method comprises:
(A) providing a composition suspected of containing the substrate;
(B) contacting the composition with EF495346 (CdPRAC, C. difficile VPI10463) [SEQ ID NO: 141] or EF175213 (TVPRAC, T. vivax [SEQ ID NO:143]; and
(C) assaying the resulting mixture for L-Pro racemization, D-Pro racemization, or both.

64. A method of detecting a substrate for a biologically active protein, wherein the method comprises:
(A) providing a composition suspected of containing the substrate;
(B) contacting the composition with at least one epimerase selected from:
EF495341 (PaHyPRE, P. aeruginosa PAK) [SEQ ID NO: 131],
EF495342 (BmHyPRE, B. melitensis 16M) [SEQ ID NO: 133],
EF495343 (BsHyPRE, B. suis 1330) [SEQ ID NO: 135],
EF495344 (BaHyPRE, B. abortus 544) [SEQ ID NO: 137], and
EF495345 (BpHyPRE, B. pseudomallei K96243) [SEQ ID NO: 139]; and
(C) assaying the resulting mixture for OH-L-Pro epimerization, OH-D-Pro epimerization, or both.

65. A method for detecting an inhibitor of a biologically active protein, wherein the method comprises:
(A) providing a composition suspected of containing an inhibitor of the biologically active protein;
(B) contacting the composition with EF495346 (CdPRAC, C. difficile VPI10463) [SEQ ID NO:141] or EF175213 (TVPRAC, T. vivax) [SEQ ID NO:143]; and
(C) assaying the resulting mixture for the inhibition of L-Pro racemization, D-Pro racemization, or both.

66. A method for detecting an inhibitor for a biologically active protein, wherein the method comprises:
(A) providing a composition suspected of containing an inhibitor of the biologically active protein;
B) contacting the composition with at least one epimerase selected from:
EF495341 (PaHyPRE, P. aeruginosa PAK) [SEQ ID NO: 131],
EF495342 (BmHyPRE, B. melitensis 16M) [SEQ ID NO: 133],
EF495343 (BsHyPRE, B. suis 1330) [SEQ ID NO: 135],
EF495344 (BaHyPRE, B. abortus 544) [SEQ ID NO: 137], and
EF495345 (BpHyPRE, B. pseudomallei K96243) [SEQ ID NO: 139]; and
(C) assaying the resulting mixture for the inhibition of OH-L-Pro epimerization, OH-D-Pro epimerization, or both.

67. A proline racemase comprising the amino acid sequence selected from SEQ ID NO: 141 and SEQ ID NO: 143.

68. A hydroxyproline epimerase comprising an amino acid sequence selected from SEQ ID NO: 131, SEQ ID NO: 133, SEQ ID NO: 135, SEQ ID NO: 137, and SEQ ID NO: 139.

69. A method for treating a patient infected with Clostridium difficile or Tripanosoma vivax, comprising administering to the patient an active molecule capable of inhibiting the proline racemase activity of a protein comprising the amino acid sequence of SEQ ID NO: 141 or SEQ ID NO: 143, wherein said method has an effect chosen from:
(a) inhibiting the growth of the Clostridium difficile or Tripanosoma vivax;
(b) preventing the Clostridium difficile or Tripanosoma vivax from evading host cell immunity; and
(c) preventing mitogen-induced proliferation of resting lymphocytes.

70. The method of claim 69, wherein the active molecule is selected from an antibody directed against the protein and pyrrole-2-carboxylic acid.

71-74. (canceled)

75. A method for treating a patient infected with Pseudomonas aeruginosa, Burkholderia pseudomallei, Brucella abortus, Brucella melitensis, or Brucella suis, comprising administering to the patient an active molecule capable of inhibiting the hydroxyproline epimerase activity of a protein comprising the amino acid sequence of SEQ ID NO: 131, SEQ ID NO: 133, SEQ ID NO: 135, SEQ ID NO: 137, or SEQ ID NO: 139, respectively, wherein said method has an effect chosen from:
(a) inhibiting the growth of Pseudomonas aeruqinosa, Burkholderia pseudomallei, Brucella abortus, Brucella melitensis, or Brucella suis;
(b) preventing Pseudomonas aeruqinosa, Burkholderia pseudomallei, Brucella abortus, Brucella melitensis, or Brucella suis from evading host cell immunity; and
(c) preventing mitogen-induced proliferation of resting lymphocytes.

76. The method of claim 75, wherein the active molecule is selected from an antibody directed against the protein, iodoacetate, and iodoacetamide.

77-80. (canceled)

81. A method for stimulating a protective immune response against Clostridium difficile, Tripanosoma vivax, Pseudomonas aeruginosa, Burkholderia pseudomallei, Brucella abortus, Brucella melitensis, or Brucella suis in a patient, comprising administering to the patient a purified mitogen comprising an amino acid sequence selected from SEQ ID NO: 141, SEQ ID NO: 143, SEQ ID NO: 131, SEQ ID NO 133, SEQ ID NO: 135, SEQ ID NO: 137, and SEQ ID NO: 139, respectively, or a portion thereof, wherein the purified mitogen or portion thereof is capable of inducing an immune response in vivo.

82. A method for stimulating a protective immune response against Clostridium difficile, Tripanosoma vivax, Pseudomonas aeruginosa, Burkholderia pseudomallei, Brucella abortus, Brucella melitensis, or Brucella suis in a patient, comprising administering to the patient a purified nucleic acid encoding a polypeptide comprising an amino acid sequence selected from SEQ ID NO: 141, SEQ ID NO: 143, SEQ ID NO: 131, SEQ ID NO 133, SEQ ID NO: 135, SEQ ID NO: 137, and SEQ ID NO: 139, respectively, or a portion thereof, wherein the polypeptide or portion thereof encoded by the nucleic acid is capable of inducing an immune response in vivo.

83. A purified antibody directed against an amino acid sequence selected from SEQ ID NO: 141, SEQ ID NO: 143, SEQ ID NO: 131, SEQ ID NO 133 SEQ ID NO: 135, SEQ ID NO: 137 and SEQ ID NO: 139 or an antigenic fragment thereof.

84. The purified antibody of claim 83, wherein the antibody is a monoclonal antibody.

85. A method for detecting Clostridium difficile, Tripanosoma vivax, Pseudomonas aeruginosa, Burkholderia pseudomallei, Brucella abortus, Brucella melitensis, or Brucella suis in a biological sample comprising:
a) bringing the biological sample into contact with an antibody according to claim 83; and
b) detecting the resulting immunocomplex.

86. A diagnostic kit for detecting Clostridium difficile, Tripanosoma vivax, Pseudomonas aeruginosa, Burkholderia pseudomallei, Brucella abortus, Brucella melitensis, or Brucella suis in a biological sample comprising:
a) an antibody according to claim 83, optionally labeled; and
b) a reagent allowing the detection of the resulting immunocomplex formed, wherein the reagent optionally carries a label.

87. A hydroxyproline-2-epimerase knock-out parasite selected from Pseudomonas aeruginosa, Burkholderia pseudomallei, Brucella abortus, Brucella melitensis, or Brucella suis, wherein the gene for the hydroxyproline-2-epimerase in the parasite has been deleted by a genetic engineering.

88. A nucleic acid sequence comprising SEQ ID NO: 130.

89. A nucleic acid element for determining whether a protein is a proline racemase or a hydroxyproline epimerase comprising SEQ ID NO: 130, wherein the presence of SEQ ID NO: 130 immediately downstream of the MIII* signature indicates a hydroxyproline epimerase.

90. (canceled)


SEQUENCE LISTING (144 sequences)

SEQ ID NO: 1 - protein, 15 aa, Artificial Sequence
  Description of Artificial Sequence Synthetic peptide motif
XXXHXXGXXXXXXXG

SEQ ID NO: 2 - protein, 32 aa, Artificial Sequence
  Description of Artificial Sequence Synthetic peptide motif
XXXXXXXXXXXXXXXXXXXXXXXXDXXXGGXX

SEQ ID NO: 3 - protein, 14 aa, Artificial Sequence
  Description of Artificial Sequence Synthetic peptide motif
DRSPXGXGXXAXXA

SEQ ID NO: 4 - protein, 14 aa, Artificial Sequence
  Description of Artificial Sequence Synthetic peptide motif
DRSPCGXGXXAXXA

SEQ ID NO: 5 - DNA, 28 bp, Artificial Sequence
  Description of Artificial Sequence Synthetic oligonucleotide
ctctcccatggggcaggaaaagcttctg

SEQ ID NO: 6 - DNA, 23 bp, Artificial Sequence
  Description of Artificial Sequence Synthetic oligonucleotide
ctgagctcgaccagatmtactgc

SEQ ID NO: 7 - DNA, 30 bp, Artificial Sequence
  Description of Artificial Sequence Synthetic oligonucleotide
gcggatcgctctccaagcgggacaggcacc

SEQ ID NO: 8 - DNA, 30 bp, Artificial Sequence
  Description of Artificial Sequence Synthetic oligonucleotide
ggtgcctgtcccgcttggagagcgatccgc

SEQ ID NO: 9 - DNA, 39 bp, Artificial Sequence
  Description of Artificial Sequence Synthetic oligonucleotide
ggctatttaaatatgtctggacataactcaattgcagcg

SEQ ID NO: 10 - DNA, 38 bp, Artificial Sequence
  Description of Artificial Sequence Synthetic oligonucleotide
cgctgcaattgagttatgtccagacatatttaaatagc

SEQ ID NO: 11 - DNA, 51 bp, Artificial Sequence
  Description of Artificial Sequence Synthetic oligonucleotide
atggataccggtggctatttaaatatgtgtggacataactcaattgcagcg

SEQ ID NO: 12 - protein, 17 aa, Artificial Sequence
  Description of Artificial Sequence Synthetic peptide
MDTGGYLNMCGHNSIAA

SEQ ID NO: 13 - DNA, 39 bp, Artificial Sequence; identical to SEQ ID NO: 9
  Description of Artificial Sequence Synthetic oligonucleotide

SEQ ID NO: 14 - DNA, 51 bp, Artificial Sequence
  Description of Artificial Sequence Synthetic oligonucleotide
atggataccggtggctatttaaatatgtctggacataactcaattgcagcg

SEQ ID NO: 15 - DNA, 51 bp, Artificial Sequence; identical to SEQ ID NO: 11
  Description of Artificial Sequence Synthetic oligonucleotide

SEQ ID NO: 16 - protein, 17 aa, Artificial Sequence
  Description of Artificial Sequence Synthetic peptide
MDTGGYLNMSGHNSIAA

SEQ ID NO: 17 - DNA, 51 bp, Artificial Sequence
  Description of Artificial Sequence Synthetic oligonucleotide
gtgatatttggcaatcgccaggcggatcgctctccatgtgggacaggcacc

SEQ ID NO: 18 - protein, 17 aa, Artificial Sequence
  Description of Artificial Sequence Synthetic peptide
VIFGNRQADRSPCGTGT

SEQ ID NO: 19 - DNA, 30 bp, Artificial Sequence; identical to SEQ ID NO: 7
  Description of Artificial Sequence Synthetic oligonucleotide

SEQ ID NO: 20 - DNA, 51 bp, Artificial Sequence; identical to SEQ ID NO: 17
  Description of Artificial Sequence Synthetic oligonucleotide

SEQ ID NO: 21 - DNA, 51 bp, Artificial Sequence
  Description of Artificial Sequence Synthetic oligonucleotide
gtgatatttggcaatcgccaggcggatcgctctccaagcgggacaggcacc

SEQ ID NO: 22 - protein, 17 aa, Artificial Sequence
  Description of Artificial Sequence Synthetic peptide
VIFGNRQADRSPSGTGT

SEQ ID NO: 23 - protein, 16 aa, Artificial Sequence
  Description of Artificial Sequence Synthetic peptide motif
XXXHXXGXXXXXXXXG

SEQ ID NO: 24 - protein, 14 aa, Artificial Sequence
  Description of Artificial Sequence Synthetic peptide motif
DRSPXGXXXXAXXA

SEQ ID NO: 25 - protein, 5 aa, Artificial Sequence
  Description of Artificial Sequence Synthetic peptide
EPRGH

SEQ ID NO: 26 - protein, 6 aa, Artificial Sequence
  Description of Artificial Sequence Synthetic peptide
EPRGSD

SEQ ID NO: 27 - protein, 6 aa, Artificial Sequence
  Description of Artificial Sequence Synthetic peptide
EPRGND

SEQ ID NO: 28 - DNA, 1598 bp, Trypanosoma cruzi
cctttttctttttaaaaacaaaaaaaattccggggggaatatggaacagggtatatgcgtaaaagtgtctgtcccaaacaaaaattttttttttccgccttcccattttttttttttttttgtgtgtttcccttgatctctcgaacagggcaggaaaagcttctgtttgaccaaaaatataaaattattaagggcgagaaaaaagaaaagaaaaaaaatcaacgagcaaacaggagagaacaccaacaaaaaagggaaattatgcgatttaagaaatcattcacatgcatcgacatgcatacggaaggtgaagcagcacggattgtgacgagtggtttgccacacattccaggttcgaatatggcggagaagaaagcatacctgcaggaaaacatggattatttgaggcgtggcataatgctggaaccacgtggtcatgatgatatgtttggagcctttttatttgaccctattgaagaaggcgctgacttgggcatggtattcatggataccggtggctatttaaatatgtgtggacataactcaattgcagcggttacggcggcagttgaaacgggaattgtgagcgtgccggcgaaggcaacaaatgttccggttgtcctggacacacctgcggggttggtgcgcggtacggcacaccttcagagtggtactgagagtgaggtgtcaaatgcgagtattatcaatgtaccctcatttttgtatcagcaggatgtggtggttgtgttgccaaagccctatggtgaagtacgggttgatattgcatttggaggcaattttttcgccattgttcccgcggagcagttgggaattgatatctccgttcaaaacctctccaggctgcaggaggcaggagaacttctgcgtactgaaatcaatcgcagtgtgaaggttcagcaccctcagctgccccatattaacactgtggactgtgttgagatatacggtccgccaacgaacccggaggcaaactacaagaacgttgtgatatttggcaatcgccaggcggatcgctctccatgtgggacaggcaccagcgccaagatggcaacactttatgccaaaggccagcttcgcatcggagagacttttgtgtacgagagcatactcggctcactcttccagggcagggtacttggggaggagcgaataccgggggtgaaggtgccggtgaccaaagatgccgaggaagggatgctcgttgtaacggcagaaattactggaaaggcttttatcatgggtttcaacaccatgctgtttgacccaacggatccgtttaagaacggattcacattaaagcagtagatctggtagagcacagaaactattggggaacacgtgcgaacaggtgctgctacgtgaagggtattgaatgaatcgtttttttttatttttattttttatttttattagtgcattattattaaattttttttttgttttggggtttcaacggtaccgcgttgggagcagggaagcgatagcggccggacaattttttgcttttattttcattttcatcttcctacccaacccccttggttccaccggtcgcggcggggtcttgtgggtggagg

SEQ ID NO: 29 - DNA, 1582 bp, Trypanosoma cruzi
gtgtgttcaacagttttgtttccttttttctctttttctctttccatcatacatacatacatacatacatatatatatctgcgtagatatgcacatgcgtatatgcgtgaagagtgtctgtcccaacattttttttttttttttgtgtgttttcccttgattcccgaacgggcaggaaaagcttctgtttgaccaaaaatataaaattattaagggcgagaaaagaaaaaaaaaatcaaccgaggagacaacaccaacaaaaaagggaaattatgcgatttaagaaatcattgacatgcatcgacatgcatacggaaggtgaagcagcacggattgtgacgagtggtttgccacacattccaggttcgaatatggcggagaagaaagcatacctgcaggaaaacatggattatttgaggcgtggcataatgctggagccacgtggtcatgatgatatgtttggagcctttttatttgaccctattgaagaaggcgctgacttgggcatcgtattcatggataccggtggctatttaaatatgtgtggacataactcaattgcagcggttacggcggcagtggaaacgggaattttgagcgtgccggcgaaggcaacaaatgttccggttgtcctggacacacctgcggggttggtgcgcggtacggcacaccttcagagtggtactgagagtgaggtgtcaaatgcgagtattatcaatgtgccctcatttttgtatcagcaggatgtggtgattgttttgccaaagccctatggtgaggtacgggttgatattgcatttggaggcaattttttcgccattgttcccgcggagcacttgggaattgatatctccgttcaaaacctctccaggctgcaggaggcaggagaacttctgcgtactgaaatcaatcgcagtgtgaaggttcagcaccctcagctgccccatattaacactgtggactgtgttgagatatacggtccgccaacgaacccggaggcaaaatacaagaacgttgtgatatttggcaatcgccaggcggatcgctctccatgtgggacaggcaccagcgccaagatggcaacactttatgccaaaggccagcttcgcatcggagagacttttgtgtacgagagcatactcggctcactcttccagggcagggtacttggggaggagcgaataccgggggtgaaggtgccggtgaccaaagatgccgaggaagggatgctcgttgtaacgacagaaattactggaaaggcttttatcatgggtttcaacaccatgctgtttgacccaacggatccgttcttaaacggattcacactaaagcggtagatctggtagagcacagaaactattggggaacacgtgcgaacaggtgctgctacgtaaagggtattgaatgaatcgtttttttttttttttttttattagtgcattatttttttttttttttgttttggggtttcaacggtaccacgttgggagcagggaaacgatagcggccggacaattttttacttttattttcattttcaccttcctacccaacccccttggttccaccggtcgcggcgggg

SEQ ID NO: 30 - protein, 423 aa, Trypanosoma cruzi [sequence omitted]

SEQ ID NO: 31 - protein, 354 aa, Trypanosoma cruzi
MRFKKSLTCIDMHTEGEAARIVTSGLPHIPGSNMAEKKAYLQENMDYLRRGIMLEPRGHDDMFGAFLFDPIEEGADLGIVFMDTGGYLNMCGHNSIAAVTAAVETGILSVPAKATNVPVVLDTPAGLVRGTAHLQSGTESEVSNASIINVPSFLYQQDVVIVLPKPYGEVRVDIAFGGNFFAIVPAEHLGIDISVQNLSRLQEAGELLRTEINRSVKVQHPQLPHINTVDCVEIYGNATNPEAKYKNVVIFGNRQADRSPCGTGTSAKMATLYAKGQLRIGETFVYESILGSLFQGRVLGEERIPGVKVPVTKDAEEGMLVVTTEITGKAFIMGFNTMLFDPTDPFLNGFTLKR

SEQ ID NO: 32 - protein, 335 aa, Clostridium sticklandii
MKFSKGIHAIDSHTMGEPTRIVVGGIPQINGETMADKKKYLEDNLDYVRTALMHEPRGHNDMFGSIITSSNNKEADFGIIFMDGGGYLNMCGHGSIGAATVAVETGMVEMVEPVTNINMEAPAGLIKAKVMVENEKVKEVSITNVPSFLYMEDAKLEVPSLNKTITFDISFGGSFFAIIHAKELGVKVETSQVDVLKKLGIEIRDLINEKIKVQHPELEHIKTVDLVEIYDEPSNPEATYKNVVIFGQGQVDRSPCGTGTSAKLATLYKKGHLKIDEKFVYESITGTMFKGRVLEETKVGEFDAIIPEITGGAYITGFNHFVIDPEDPLKYGFTV

SEQ ID NO: 33 - protein, 354 aa, Homo sapiens
MESALAVPWLPPHDPGTPVLSVVDMHTGGEPLRIVLAGCPEVSGPTLLAKRRYMRQHLDHVRRRLMFEPRGHRDMYGAVLVPSELPDAHLGVLFLHNEGYSSMCGHAVLALGRFALDFGLVPAPPAGTREARVNIHCPCGLVTAFVACEDGRSHGPVRFHSVPAFVLATDLMVDVPGHGKVMVDIAYGGAFYAFVTAEKLGLDICSAKTRDLVDAASAVTEAVKAQFKINHPDSEDLAFLYGTILTDGKDAYTKEPTTNICVFADEQVDRSPTGSGVTARIALQYHKGLLELNQMRAFKSSATGSVFTGKAVREAKCGDFKAVIVEVSGQAHYTGTASFIIEDDDPLRDGFLLK

SEQ ID NO: 34 - protein, 354 aa, Homo sapiens
MESALAVPRLPPHDPGTPVLSVVDMHTGGEPLRIVLAGCPEVSGPTLLAKRRYMRQHLDHVRRRLMFEPRGHRDMYGAVLVPSELPDAHLGVLFLHNEGYSSMCGHAVLALGRFALDFGLVPAPPAGTREARVNIHCPCGLVTAFVACEDGRSHGPVRFHSVPAFVLATDLMVDVPGHGKVMVDIAYGGAFYAFVTAEKLGLDICSAKTRDLVDAASAVTEAVKAQFKINHPDSEDLAFLYGTILTDGKDAYTKEPTTNICVFADEQVDRSPTGSGVTARIALQYHKGLLELNQMRAFKSSATGSVFTGKAVREAKCGDFKAVIVEVSGQAHYTGTASFIIEDDDPLRDGFLLK

SEQ ID NO: 35 - protein, 354 aa, Mus musculus
MEAALAVTRLPPNDPRTPALSVVDMHTGGEPLRIVHAGCPEVAGPTLLAKRRYMRQHLDYIRRRLVFEPRGHRDMYGAILVPSELPDAHLGVLFLHNEGYSSMCGHAVLALGRFALDFGLVPAPPKGAREAQVNIHCPCGLVTAFVECEGGRSCGPVRFHSVPAFVLASDLTVDVPGHGKVLVDIAYGGAFYAFVSAEKLGLDVCSAKTRDLVDAASALTGAVKAQFKINHPESEDLGFLYGSILTDGKDAYSEEATTNICVFADEQVDRSPTGSGVTARIALQYHKGLLQLNQTRAFKSSATGSVFTGCAVREAKCGDFKAVIVEVAGQAHYTGTANLTVEDGDPLRDGFLLK

SEQ ID NO: 36 - protein, 354 aa, Mus musculus
MEAALAVTRLPPHDSRTPALSVVDMHTGGEPLRIVHAGCPEVAGPTLLAKRRYMRQHLDYIRRRLVFEPRGHRDMYGAILMPSELPDAHLGVLFLHNEGYSSMCGHAVLALGRFALDFGLVPAPPEGAREAQVNIHCPCGLVTAFVECEGGRSCGPVRFHSVPAFVLASDLTVDVPGHGKVLVDIAYGGAFYAFVSAEKLGLDVCSAKTRDLVDAASALTGAVKAQFKINHPESEDLGFLYGSILTDGKDAYSEEATTNICVFADEQVDRSPTGSGVTARIALQYHKGLLQLNQTRAFKSSATGSVFTGCAVREAKCGDFKAVIVEVAGQAHYTGTANLTVEDGDPLRDGFLLK

SEQ ID NO: 37 - protein, 333 aa, Rhizobium loti
MAKKSFFCIDGHTCGNPVRLVAGGGPLLEGSTMMERRAHFLAEYDWIRTGLMFEPRGHDVMSGSILYPPTREDCDIAILFIETSGCLPMCGHGTIGTVTMAIEHGLIKPKTPGVLRLDTPAGLVIAEYKQVGEYVEEVRITNVPSFLHAEGLTVECPGLGEITVDVAYGGNFYAIVEPQENYRDMADHSAGDLIAWSPVVRQRLNEKYSFVHPENPGINRLSHMLWTGKPTVEGADARNAVFYGDKAIDRSPCGTGTSARMAQLHAKGKLKAGDAFVHESIIGSLFKGKVEKEVTVAGKPAIIPSIGGWARLTGLNTIFIDDRDPFAHGFVVT

SEQ ID NO: 38 - protein, 333 aa, Brucella melitensis
MARHSFFCVDGHTCGNPVRLVAGGGPNLNGSTMMEKCAHFLAEYDWIRTGLMFEPRGHDMMSGSILYPPTRPDCDVAVLFIETSGCLPMCGHGTIGTVTMAIEQGLVTPKTPGKLNLDTPAGLVAIEYEQDGQYVERVRLTNVPAFLYAEGLEVECPDLGPIKVDVAYGGNFYAIVEPQENYTDMDDYSALQLIAWSPVLRQRLNEKYKFQHPELPDINRLSHILWTGKPKHPQAHARNAVFYGDKAIDRSPCGTGTSARMAQLAAKGKLKPGDEFIHESIIGSLFHGRVERAAEVAGRPAIVPSIAGWARMTGYNTIFIDDRDPFAHGFSAA

SEQ ID NO: 39 - protein, 333 aa, Rhizobium meliloti
MATHTFSCIDGHTCGNPVRLVSGGGPRLEGANMLEKRAHFLKEFDWIRTGLMFEPRGHDMMSGSILYPPTRPDCDVAVLFIETSGCLPMCGHGTIGTITMGIENGLITPREPGKLSIDAPAGKVDITYRQEGRFVEEVRLTNVPSFLYAEGLAAEVEGLGEIVVDVAYGGNFYAIVEPQKNFRDMADHTAGELVGWSPKLRAALNAKYEFVHPEHPEIRGLSHIQWTGKPTQPEAHARNAVFYGEKAIDRSPCGTGTSARIAQLAAKGKLKVGDEFVHESIIGSLFKGRVEAAAKVADRDAIIPSIAGWARMTGINTIFIDDRDPFAHGFVVR

SEQ ID NO: 40 - protein, 332 aa, Agrobacterium tumefaciens
MRHSFFCIDSHTCGNPVRLVAGGGPLLPHLPISERRDLFVRNHDWVRQALMFEPRGHDIMSGAVIYPAYRDDCDFAVIFIEVSGCLPMCGAGTIGLVTAAIEEGLVTPRIPGRLSIETPAGKVDIQYDKPGEFVESVRIFNVASYLHAADVEVNVPGLGKLVVDIAYGGNYYAVIEPQVGWPGLDGMTAGDVVDLSQKLRDALGTICDPVHPDDERIRGVHHAIWCDRPVSAEADGRGAVFYGDKAIDRSPGGTGTSARMAQLHGKGRLKAGETFRQESLIGTIFEGKVEEETTVGSFSGIRPSIGGWARIIGHNTIFVDDRDPLAHGFQVR

SEQ ID NO: 41 - protein, 312 aa, Xanthomonas campestris
MHTIDVIDSHTAGEPTRVVLAGFPDLGDGDLAQCRERFRSDFDHWRSAIACEPRGSDTMVGALLLPPRDPSACTGVIFFNNVGYLGMCGHGTIGVVRTLAELGRIAPGQHRIETPVGTVGVALADDGTVSIDNVESYRHAAGVEVDVPGHGRVRGDVAWGGNWFFITEQAPCALGLAQQRELTAYTEAIRLALEAAGITGEAGGEIDHIEISGVAPDGSGAARNFVLCPGLAYDRSPCGTGTSAKLACLAADGKLAEGERWLQQGILGSAFEGSYRHSGRGIAPRISGHAFITARSQLLIDPADPFAWGIVA

SEQ ID NO: 42 - protein, 312 aa, Xanthomonas axonopodis
MHTIDVIDSHTAGEPTRVVLSGFPDLGDGDLAQCRERFRSEFDHWRSAIACEPRGSDTMVGALLLPPRDPSACTGVIFFNNVGYLGMCGHGTIGVVRTLAELGRIAPGQHRIETPVGTVGVELADDGTVSVDNVESYRFASGVEVEVPGHGRVCGDVAWGGNWFFITEHAPCALDLAHQRELTAYTEAIRLALEAAGITGEAGGEIDHIEVNGAAPDGSGVARNFVLCPGLAYDRSPCGTGTSAKLACLAADGKLAEGERWVQQGILGSAFEGNYRLSGRGIAPRISGRAYITARAQLVIDPADPFAWGIVA

SEQ ID NO: 43 - protein, 314 aa, Pseudomonas aeruginosa
MQRIRIIDSHTGGEPTRLVIGGFPDLGQGDMAERRRLLGERHDAWRAACILEPRGSDVLVGALLCAPVDPEACAGVIFFNNSGYLGMCGHGTIGLVASLAHLGRIGPGVHRIETPVGEVEATLHEDGSVSVRNVPAYRYRRQVSVEVPGIGRVSGDIAWGGNWFFLVAGHGQRLAGDNLDALTAYTVAVQQALDDQDIRGEDGGAIDHIELFADDPHADSRNFVLCPGKAYDRSPCGTGTSAKLACLAADGKLLPGQPWRQASVIGSQFEGRYEWLDGQPGGPIVPTIRGRAHVSAEATLLLADDDPFAWGIRR

SEQ ID NO: 44 - protein, 344 aa, Pseudomonas aeruginosa
MRSQRIVHIVSCHAEGEVGDVIVGGVAAPPGATLWEQSRWIARDQDLRNFVLNEPRGGVFRHANLLVPAKDPRAQMGWIIMEPADTPPMSGSNSLCVATVLLDSGILPMREPLTRLLLEAPGGLIEARAECRDGKAERVEIRNVPSFADRLDAWIEVEGLGSLQVDTAYGGDSFVIADARRLGFALRADEAAELVATGLKITHAANEQLGFRHPTNPDWDHLSFCQLAAPPERRDGVLGANNAVVIRPGKIDRSPCGTGCSARMAVLQAKGQLRVGERFVGRSIIGSEFHCHIESLTELGGRPAILPCLSGRAWITGIHQYLLDPDDPWPQGYRLSDTWPGGHC

SEQ ID NO: 45 - protein, 342 aa, Brucella melitensis
MRSTKVIHIVGCHAEGEVGDVIVGGVAPPPGETVWEQSRFIANDETLRNFVLNKPRGGVFRHVNLLVPPKDPRAQMGFIIMEPADTPPMSGSNSICVSTVLLDSGIIAMQEPVTHMVLEAPGGIIEVEAECRNGKAERISVRNVPSFADRLDAPLDVTGLGTIMVDTAYGGDSFVIVDAAQIGMKIEPGQARELAEIGVKITKAANEQLGFRHPERDWRHISFCQITEPVTREGDVLTGVNTVAIRPAKFDRSPTGTGCSARMAVLHAKGQMKAGERFIGKSVLGTEFHCRLDKVLELGGKPAISPIISGRAWVTGTSQLMLDPSDPFPHGYRLSDTWPRDE

SEQ ID NO: 46 - protein, 342 aa, Agrobacterium tumefaciens
MRSIKTVHVISAHAEGEVGDVIVGGVKPPPGETIWEQSRFIARDETLRNFVLNEPRGGVFRHVNLLVPPKHPDADAAFIIMEPEDTPPMSGSNSICVSTVLLDGGIVPMQEPETHMLLEAPGGLVKVRAECRNGKAERIFVQNLPSFAAKLDAELEVEGLGKLKVDTAYGGDSFVIVDAEAMGFSLKPEEAHEIARLGVRITNAANKALGFDHPENPDWRHFSFCLFAGKVERTAEGLRAGAAVAIQPGKVDRSPTGTALSARMAVLHARGEMKEGETLTAVSLIGSTFTGRILGTTTVGDRPAILPEISGRGWITGIHQHMLDPSDPWPEGYRLTDTWGAR

SEQ ID NO: 47 - protein, 342 aa, Rhizobium meliloti
MRSTKTIHVISAHAEGEVGDVIVGGVAPPPGDTIWEQSRWIAREQTLRNFVLNEPRGGVFRHVNLLVPPKHPDADAAFIIMEPEDTPPMSGSNSICVSTVLLDSGILPMKEPVTEITLEAPGGLVRVRAECRDGKAERIFVENLPSFAERLDAKLEVEGLGTLTVDTAYGGDSFVIVDAAAMGFALKPDEAHDIARLGVRITNAANAKLGFHHPENPDWRHFSFCLFAGPVERTAEGLRAGAAVAIQPGKVDRSPTGTALSARMAVLHARGQMGLSDRLTAVSLIGSTFSGRILGTTEVGGRPAVLPEISGRAWITGTHQHMLDPSDPWPEGYRLTDTWGAR

SEQ ID NO: 48 - protein, 346 aa, Rhizobium loti
MRSKTSIRVVGCHAEGEVGDVIIGGVLPPAGRTMMDKMITMERDHDHIRRMLICEPRGSVARHVNLLVPSTREDCAAGAIIMEPTEYPPMSGSNTICVATVLLETGMVPMQEPETRFKLDMPGGVIEVRAQCRDGKCVSITLRNAPAFVDRLDASIEVEGLGTLTVDIAYGGMFYAIVDAKALGFSIAPDEARELAVAGEKIRRAAREQLDVVHPQFDHVRGVSIVQFAMPFQGPGNVTRNTCIVSPGRSDRSPTGTGTSARMAVLQARGLMGVGDVLIHESIIGSRFTGRIVELAEIAGRKAIVPEITGRAWITGEHSYYLDPTDPYPQGYVLSDTWGTSTSVKQ

SEQ ID NO: 49 - protein, 339 aa, Xanthomonas axonopodis
MRWSKQFSVVDCHAEGEVGKVIVGGVGNIPGTTMFEKKLYLEQHRDDIRKLVLQEPRGATWHNANIILPPSHPEASMGFVILEATEYPAMSGSNTICVATVLLETGILPMQEPITDLVLEAPAGLIRVRCDCKDGKVTRVKLVNQPAFVYHLDAKVEVAGIGTVSADIAFGGMTFALVDASSLGFEIVPAEARELCEYGQKIKAAAAEQLDVAFPGNPDMPGITMTQFTGPLSRADGKFFSRNTTIVSPGRCDRSPCGAGSSARLAALHAKGVLAKGDTLVHESIIGSRFECGIEDMSNVGDYPAVVPSIAGQAWISGLSQLGLDPSDPYAEGFTLADR

SEQ ID NO: 50 - protein, 333 aa, Streptomyces coelicolor
MRSTVCYHAVDSHTEGMPTRVITGGVGVLPGATMFERRQRFVAERDHLRTLLMCEPRGHASMSGAILQPPTRPDADYGVLFIEVSGCLPMCGHGTIGVATVLVETGMVEVTEPETTVRLDTPAGLVTARVRVRDGHAESVTLENVASYSHALDQVVDVPGHGEVRYDIAYGGNFYAFVRTDDLGIPFERAHKQPLLDAGLAVMDAINKQNPVSHPENPDIDVCHHVYLEAPGSTAEHSRHAMAIHPGWFDRSPCGTGTSARMAQLHARGLLPAGRDFVNESFIGSRFVGRVLGETTVGGRPAVLPSVTGRAWITGTAQYLLDPSDPYPAGFTL

SEQ ID NO: 51 - protein, 345 aa, Agrobacterium tumefaciens
MRWKRTLQLLDVHCEGEIGRVVTGGAPKIPGNTVAEQLHWMNTDPQGEALRRFLTLEPRGTPMGSVDLLLPPKHPDAHAAFVILQPDQAHASSGSNSICATTALLESGMVEMQEPETVIILETAAGLVKATATCRDGRCEKVKLTMVPSFVHELDVSIDTPEWGRVTMDISYGGIFYALVDVRQIGLTIEKANAAKLVAAGMTLKDLVNREMTVVHPEIPAISGVAYVMFRDVDADGSIRTCTTMWPGRADRSPCGTGNSANLATLYARGKVKVGDEYKSRSIIGSEFDVGLSAVTEVAGRPAVIPTIAGRGFTFGLHQVGLDPFDPLGDGFAMTDVWGPEAGNI

SEQ ID NO: 52 - protein, 354 aa, Trypanosoma cruzi
MRFKKSLTCIDMHTEGEAARIVTSGLPHIPGSNMAEKKAYLQENMDYLRRGIMLEPRGHDDMFGAFLFDPIEEGADLGIVFMDTGGYLNMCGHNSIAAVTAAVETGILSVPAKATNVPVVLDTPAGLVRGTAHLQSGTESEVSNASIINVPSFLYQQDVVIVLPKPYGEVRVDIAFGGNFFAIVPAEHLGIDISVQNLSRLQEAGELLRTEINRSVKVQHPQLPHINTVDCVEIYGPPTNPEAKYKNVVIFGNRQADRSPCGTGTSAKMATLYAKGQLRIGETFVYESILGSLFQGRVLGEERIPGVKVPVTKDAEEGMLVVTTEITGKAFIMGFNTMLFDPTDPFLNGFTLKR

SEQ ID NO: 53 - DNA, 15 bp, Artificial Sequence
  Description of Artificial Sequence Synthetic oligonucleotide
tctccatgtgggaca

SEQ ID NO: 54 - protein, 5 aa, Artificial Sequence
  Description of Artificial Sequence Synthetic peptide
SPCGT

SEQ ID NO: 55 - DNA, 15 bp, Artificial Sequence
  Description of Artificial Sequence Synthetic oligonucleotide
tctccaagcgggaca

SEQ ID NO: 56 - protein, 5 aa, Artificial Sequence
  Description of Artificial Sequence Synthetic peptide
SPSGT

SEQ ID NO: 57 - protein, 146 aa, Trypanosoma cruzi
GEVRVDIAFGGNFFAIVPAEQLGIDISVQNLSRLQEAGELLRTEINRSVKVQHPQLPHINTVDCVEIYGPPTNPEANYKNVVIFGNRQADRSPCGTGTSAKMATLYAKGQLRIGETFVYESILGSLFQGRVLGEERIPGVKVPVTK

SEQ ID NO: 58 - protein, 146 aa, Bacillus anthracis
GTVEADIAYGGNFYAIIDAKSVGLELVPEHASTIIDKAIHIRNIINERFEIIHPEYSFIRGLTHVEFYTDPTHESAHVKNTVVVPPGGIDRSPCGTGTSAKLAVLYANQKIEMNEEFVHESIVGSLFKGCVINTTNVANMEAVVTK

SEQ ID NO: 59 - protein, 117 aa, Trypanosoma cruzi
MRFKKSFTCIDMHTEGEAARIVTSGLPHIPGSNMAEKKAYLQENMDYLRRGIMLEPRGHDDMFGAFLFDPIEEGADLGMVFMDTGGYLNMCGHNSIAAVTAAVETGIVSVPAKATNV

SEQ ID NO: 60 - protein, 117 aa, Bacillus anthracis
MRTQKVFTTIDTHTGGNPTRTLISGLPKLLGETMAEKMLHMKKEYDWIRKLLMNEPRGHDVMSGALLTDPCHPDADIGVIYIETGGYLPMCGHDTIGVCTALIESGLIPVVEPITSL

SEQ ID NO: 61 - protein, 145 aa, Trypanosoma cruzi
EVRVDIAFGGNFFAIVPAEQLGIDISVQNLSRLQEAGELLRTEINRSVKVQHPQLPHINTVDCVEIYGPPTNPEANYKNVVIFGNRQADRSPCGTGTSAKMATLYAKGQLRIGETFVYESILGSLFQGRVLGEERIPGVKVPVTK

SEQ ID NO: 62 - protein, 145 aa, Bacillus anthracis
EFQVDIAFGGAFYAVVDSKEFGLKVDFKDLSAIQQWGGKIKHYIESKMEVKHPLEEGLKGIYGVIFSDDPKGEGATLRNVTIFADGQVDRSPCGTGTSARIATLFEKGILQKGEIFIHECITDGEFEGEVLSVTAVHTYEAVVPK

SEQ ID NO: 63 - protein, 113 aa, Trypanosoma cruzi
MRFKKSFTCIDMHTEGEAARIVTSGLPHIPGSNMAEKKAYLQENMDYLRRGIMLEPRGHDDMFGAFLFDPIEEGADLGMVFMDTGGYLNMCGHNSIAAVTAAVETGIVSVPAK

SEQ ID NO: 64 - protein, 113 aa, Bacillus anthracis
MKVSKVYTTIDAHVAGEPLRIITGGVPEIKGETQLERRWYCMEHLDYLREVLMYEPRGHHGMYGCIITPPASAHADFGVLFMHNEGWSTMCGHGIIAVITVGIETGMFETKQK

SEQ ID NO: 65 - protein, 138 aa, Trypanosoma cruzi
YGEVRVDIAFGGNFFAIVPAEQLGIDISVQNLSRLQEAGELLRTEINRSVKVQHPQLPHINTVDCVEIYGPPTNPEANYKNVVIFGNRQADRSPCGTGTSAKMATLYAKGQLRIGETFVYESILGSLFQGRVLGEERI

SEQ ID NO: 66 - protein, 138 aa, Clostridium botulinum
YGKLTLDISFGGSFFAMVDAEKVGIDISPANSQKLNDLGMKIVHAVNEQVEIKHPVLEHIKTVDLCEFYGPAKSEDADVQNVVVFGQGQVDRSPCGTGTSAKMALLYAQGKMKVGEEIVNESIICTKFKGKILEETKV

SEQ ID NO: 67 - protein, 118 aa, Trypanosoma cruzi
IMRFKKSFTCIDMHTEGEAARIVTSGLPHIPGSNMAEKKAYLQENMDYLRRGIMLEPRGHDDMFGAFLFDPIEEGADLGMVFMDTGGYLNMCGHNSIAAVTAAVETGIVSVPAKATNV

SEQ ID NO: 68 - protein, 118 aa, Clostridium botulinum
IMRAIKTIQTIESHTMGEPTRIVIGGLPKVPGKTMAEKMEYLEENNDSLRTMLMSEPRGHNDMFGAIYTEPADETADLGIIFMDGGGYLNMCGHGSIGAATCAVEMGIVKVEEPYTNI

SEQ ID NO: 69 - protein, 224 aa, Trypanosoma cruzi
ADLGIVFMDTGGYLNMCGHNSIAAVTAAVETGILSVPAKATNVPVVLDTPAGLVRGTAHLQSGTESEVSNASIINVPSFLYQQDVVIVLPKPYGEVRVDIAFGGNFFAIVPAEHLGIDISVQNLSRLQEAGELLRTEINRSVKVQHPQLPHINTVDCVEIYGNATNPEAKYKNVVIFGNRQADRSPCGTGTSAKMATLYAKGQLRIGETFVYESILGSLFQGRV

SEQ ID NO: 70 - protein, 218 aa, Clostridium botulinum
ADLGIIFMDGGGYLNMCGHGSIGAATCAVEMGIVKVEEPYTNIKLEAPAGMINARVKVEDGKAKETSIVNVPAFLYKKDVEIDVPDYGKLTLDISFGGSFFAMVDAEKVGIDISPANSQKLNDLGMKIVHAVNEQVEIKHPVLEHIKTVDLCEFYGPAKSEDADVQNVVVFGQGQVDRSPCGTGTSAKMALLYAQGKMKVGEEIVNESIICTKFKGKI

SEQ ID NO: 71 - protein, 72 aa, Trypanosoma cruzi
PTNPEANYKNVVIFGNRQADRSPCGTGTSAKMATLYAKGQLRIGETFVYESILGSLFQGRVLGEERIPGVKV

SEQ ID NO: 72 - protein, 72 aa, Aspergillus fumigatus
PDDVQGAETGLCYFAENQIDRSPTGSCVIARMALAYAKGLRSLGQRWAYNSLVSNRFGTGAFSAEIVEEVTI

SEQ ID NO: 73 - protein, 34 aa, Trypanosoma cruzi
MAEKKAYLQENMDYLRRGIMLEPRGHDDMFGAFL

SEQ ID NO: 74 - protein, 34 aa, Aspergillus fumigatus
LLEQRDQAKQHHDHIRKCLMLEPRGHNGMYGAII

SEQ ID NO: 75 - protein, 29 aa, Trypanosoma cruzi
CIDMHTEGEAARIVTSGLPHIPGSNMAEK

SEQ ID NO: 76 - protein, 29 aa, Aspergillus fumigatus
CIDMHTTGEPTRIIYSGFPPLSGTLLEQR

SEQ ID NO: 77 - protein, 27 aa, Trypanosoma cruzi
VDIAFGGNFFAIVPAEQLGIDISVQNL

SEQ ID NO: 78 - protein, 27 aa, Aspergillus fumigatus
LDISYGGAFYAIVQASELGFSGGLRDL

SEQ ID NO: 79 - protein, 20 aa, Trypanosoma cruzi
SSIGSNKKAPNISSPRGSSI

SEQ ID NO: 80 - protein, 20 aa, Aspergillus fumigatus
SSVSGRMMAPYIPLPRGSSI

SEQ ID NO: 81 - protein, 146 aa, Trypanosoma cruzi; identical to SEQ ID NO: 57

SEQ ID NO: 82 - protein, 146 aa, Clostridium difficile
GTVKFDISFGGSFFAIIHASQLGLKIEPQNAGKLTELAMKLRDIINEKIEIQHPTLAHIKTVDLVEIYDEPTHPEATYKNVVIFGQGQVDRSPCGTGTSAKLATLHAKGELKVGEKFVYESILGTLFKGEIVEETKVADFNAVVPK

SEQ ID NO: 83 - protein, 117 aa, Trypanosoma cruzi; identical to SEQ ID NO: 59

SEQ ID NO: 84 - protein, 117 aa, Clostridium difficile
MKFSRSIQAIDSHTAGEATRIVVGGIPNIKGNSMPEKKEYLEENLDYLRTAIMLEPRGHNDMFGSVMTQPCCPDADFGIIFMDGGGYLNMCGHGTIGAMTAAIETGVVPAVEPVTHV

SEQ ID NO: 85 - protein, 139 aa, Trypanosoma cruzi
GEVRVDIAFGGNFFAIVPAEQLGIDISVQNLSRLQEAGELLRTEINRSVKVQHPQLPHINTVDCVEIYGPPTNPEANYKNVVIFGNRQADRSPCGTGTSAKMATLYAKGQLRIGETFVYESILGSLFQGRVLGEERIPG

SEQ ID NO: 86 - protein, 139 aa, Brucella suis
GPIKVDVAYGGNFYAIVEPQENYTDMDDYSALQLIAWSPVLRQRLNEKYKFQHPELPDINRLSHILWTGKPKHPQAHARNAVFYGDKAIDRSPCGTGTSARMAQLAAKGKLKPGDEFIHESIIGSLFHGRVERAAEVAG

SEQ ID NO: 87 - protein, 106 aa, Trypanosoma cruzi
KKSFTCIDMHTEGEAARIVTSGLPHIPGSNMAEKKAYLQENMDYLRRGIMLEPRGHDDMFGAFLFDPIEEGADLGMVFMDTGGYLNMCGHNSIAAVTAAVETGIVS

SEQ ID NO: 88 - protein, 106 aa, Brucella suis
RHSFFCVDGHTCGNPVRLVAGGGPNLNGSTMMEKRAHFLAEYDWIRTGLMFEPRGHDMMSGSILYPPTRPDCDVAVLFIETSGCLPMCGHGTIGTVTMAIEQGLVT

SEQ ID NO: 89 - protein, 109 aa, Trypanosoma cruzi
MRFKKSFTCIDMHTEGEAARIVTSGLPHIPGSNMAEKKAYLQENMDYLRRGIMLEPRGHDDMFGAFLFDPIEEGADLGMVFMDTGGYLNMCGHNSIAAVTAAVETGIVS

SEQ ID NO: 90 - protein, 109 aa, Rhodobacter sphaeroides
MRVQDVYNVIYTHTEGEPLCIIYSGVPYPAGSTILEKRAFLEENYDWLRKALMREPRGHADMFGVFLTPPSSRDYDAGLIYIDGKEYSHMCGHGTIAVAMAMVANGLVA

SEQ ID NO: 91 - protein, 47 aa, Trypanosoma cruzi
KVQHPQLPHINTVDCVEIYGPPTNPEANYKNVVIFGNRQADRSPCGT

SEQ ID NO: 92 - protein, 47 aa, Rhodobacter sphaeroides
KSSTPTEAHINNLNFVTLWHKPPSRGWLYKNVHCFLEGQLDRLPGGT

SEQ ID NO: 93 - protein, 118 aa, Trypanosoma cruzi
RFKKSFTCIDMHTEGEAARIVTSGLPHIPGSNMAEKKAYLQENMDYLRRGIMLEPRGHDDMFGAFLFDPIEEGADLGMVFMDTGGYLNMCGHNSIAAVTAAVETGIVSVPAKATNVPV

SEQ ID NO: 94 - protein, 118 aa, Burkholderia pseudomallei
RDMKHIHIIDSHTGGEPTRVVVSGFPALGGGTMAERLAVLAREHDRYRAACILEPRGSDVLVGALLCEPVSAGAAAGVIFFNNAGYLGMCGHGTIGLVRTLHHMGRIGPGVHRIETPV

SEQ ID NO: 95 - protein, 63 aa, Trypanosoma cruzi
NPEANYKNVVIFGNRQADRSPCGTGTSAKMATLYAKGQLRIGETFVYESILGSLFQGRVLGEE

SEQ ID NO: 96 - protein, 63 aa, Burkholderia pseudomallei
DPEYDSRSFVLCPGHAYDRSPCGTGTSAKLACLAADGKLAAGVTWRQASVIGSVFSASYAAAE

SEQ ID NO: 97 - protein, 118 aa, Trypanosoma cruzi; identical to SEQ ID NO: 93

SEQ ID NO: 98 - protein, 118 aa, Burkholderia mallei; identical to SEQ ID NO: 94

SEQ ID NO: 99 - protein, 63 aa, Trypanosoma cruzi; identical to SEQ ID NO: 95

SEQ ID NO: 100 - protein, 63 aa, Burkholderia mallei
DPEYDSRSFVLCPGHAYDRSPCGTGTSAKLACLAADGKLVAGVTWRQASVIGSVFSASYAAAE

SEQ ID NO: 101 - protein, 115 aa, Trypanosoma cruzi
KSFTCIDMHTEGEAARIVTSGLPHIPGSNMAEKKAYLQENMDYLRRGIMLEPRGHDDMFGAFLFDPIEEGADLGMVFMDTGGYLNMCGHNSIAAVTAAVETGIVSVPAKATNVPV

SEQ ID NO: 102 - protein, 115 aa, Pseudomonas putida
KQIHVIDSHTGGEPTRLVMKGFPQLRGRSMAEQRDELRELHDRWRRACLLEPRGNDVLVGALYCPPVSADATCGVIFFNNAGYLNMCGHGTIGLVASLQHMGLITPGVHKIDTPV

SEQ ID NO: 103 - protein, 58 aa, Trypanosoma cruzi
NPEANYKNVVIFGNRQADRSPCGTGTSAKMATLYAKGQLRIGETFVYESILGSLFQGR

SEQ ID NO: 104 - protein, 58 aa, Pseudomonas putida
DPNADSRNFVMCPGKAYDRSPCGTGTSAKLACLAADGKLAEGQTWVQASITGSQFHGR

SEQ ID NO: 105 - protein, 180 aa, Trypanosoma cruzi
SGLPHIPGSNMAEKKAYLQENMDYLRRGIMLEPRGHDDMFGAFLFDPIEEGADLGIVFMDTGGYLNMCGHNSIAAVTAAVETGIVSVPAKATNVPVVLDTPAGLVRGTAHLQSGTESEVSNASIINVPSFLYQQDVVVVLPKPYGEVRVDIAFGGNFFAIVPAEQLGIDISVQNLSRLQE

SEQ ID NO: 106 - protein, 164 aa, Leishmania major
TGFPELAGETIADKLDNLRTQHDQWRRACLLEPRGNDVLVGALYCAPVSADATCGVIFFNNAGYLGMCGHGTIGLVASLHHLGRIAPGVHKIDTPVGPVSATLHADGAVTLRNVPAYRYRQQVPVDVPGHGRVYGDIAWGGNWFFLVSDHGQALQMDNVEALTD

SEQ ID NO: 107 - protein, 68 aa, Trypanosoma cruzi
PTNPEANYKNVVIFGNRQADRSPCGTGTSAKMATLYAKGQLRIGETFVYESILGSLFQGRVLGEERIP

SEQ ID NO: 108 - protein, 68 aa, Leishmania major
PTTPTPTATSSCAQGKAYDRSPCGTGTNAKLACLAGDSKLAAGEPWLQVTITCRQFKRSYQWECKRVP

SEQ ID NO: 109 - protein, 28 aa, Trypanosoma cruzi
VTAEITGKAFIMGFNTMLFDPTDPFKNG

SEQ ID NO: 110 - protein, 27 aa, Leishmania major
VPPSITRRAYMTADSTLLIDQDPFAWG

SEQ ID NO: 111 - protein, 182 aa, Trypanosoma cruzi
VTSGLPHIPGSNMAEKKAYLQENMDYLRRGIMLEPRGHDDMFGAFLFDPIEEGADLGIVFMDTGGYLNMCGHNSIAAVTAAVETGIVSVPAKATNVPVVLDTPAGLVRGTAHLQSGTESEVSNASIINVPSFLYQQDVVVVLPKPYGEVRVDIAFGGNFFAIVPAEQLGIDISVQNLSRLQE

SEQ ID NO: 112 - protein, 166 aa, Leishmania major
VMTGFPELAGETIADKLDNLRTQHDQWRRACLLEPRGNDVLVGALYCAPVSADATCGVIFFNNAGYLGMCGHGTIGLVASLHHLGRIAPGVHKIDTPVGPVSATLHADGAVTLRNVPAYRYRQQVPVDVPGHGRVYGDIAWGGNWFFLVSDHGQALQMDNVEALTD

SEQ ID NO: 113 - protein, 142 aa, Trypanosoma cruzi
RIVTSGLPHIPGSNMAEKKAYLQENMDYLRRGIMLEPRGHDDMFGAFLFDPIEEGADLGIVFMDTGGYLNMCGHNSIAAVTAAVETGILSVPAKATNVPVVLDTPAGLVRGTAHLQSGTESEVSNASIINVPSFLYQQDVVI

SEQ ID NO: 114 - protein, 137 aa, Trypanosoma brucei
RIITGGVPEIKGETQLERRAYCMEHLDYLREILMYEPRGHHGMYGCIITPPASAHADFGVLFMHNEGWSTMCGHGIIAVITVGIETGMFEVKGEKQNFIIDSPAGEVIAYAKYNGSEVESVSFENVPSFVYKKDVPI

SEQ ID NO: 115 - protein, 140 aa, Trypanosoma cruzi
VTSGLPHIPGSNMAEKKAYLQENMDYLRRGIMLEPRGHDDMFGAFLFDPIEEGADLGIVFMDTGGYLNMCGHNSIAAVTAAVETGILSVPAKATNVPVVLDTPAGLVRGTAHLQSGTESEVSNASIINVPSFLYQQDVVI

SEQ ID NO: 116 - protein, 135 aa, Trypanosoma brucei
ITGGVPEIKGETQLERRAYCMEHLDYLREILMYEPRGHHGMYGCIITPPASAHADFGVLFMHNEGWSTMCGHGIIAVITVGIETGMFEVKGEKQNFIIDSPAGEVIAYAKYNGSEVESVSFENVPSFVYKKDVPI

SEQ ID NO: 117 - protein, 103 aa, Trypanosoma cruzi
FGGNFFAIVPAEQLGIDISVQNLSRLQEAGELLRTEINRSVKVQHPQLPHINTVDCVEIYGPPTNPEANYKNVVIFGNRQADRSPCGTGTSAKMATLYAKGQL

SEQ ID NO: 118 - protein, 95 aa, Trypanosoma congolense
WGGNWFFLVSDHGHELQMDNVEALTDYTWAMLNALEAQGIRGADGALIDHIELFADDAHADSRNFVMCPGKAYDRSPCGTGTSAKLACLAADAKL

SEQ ID NO: 119 - protein, 55 aa, Trypanosoma cruzi
NVPSFLYQQDVVVVLPKPYGEVRVDIAFGGNFFAIVPAEQLGIDISVQNLSRLQE

SEQ ID NO: 120 - protein, 52 aa, Trypanosoma congolense
HVPAYRYRKQVPVEVPGHGVVLGDIAWGGNWFFLVSDHGHELQMDNVEALTD

SEQ ID NO: 121 - protein, 117 aa, Trypanosoma cruzi
LPKPYGEVRVDIAFGGNFFAIVPAEQLGIDISVQNLSRLQEAGELLRTEINRSVKVQHPQLPHINTVDCVEIYGPPTNPEANYKNVVIFGNRQADRSPCGTGTSAKMATLYAKGQLR

SEQ ID NO: 122 - protein, 116 aa, Trypanosoma vivax
LPHPYGKYAVISFGGSFFALIDAAQLQLTVDKGHLSTLQHVGGLLRDTLNRNVSVQHPQLPHINRIDCVEIYDPPTNPAASCKNVVIFGNSQVDRSPCGTGTCAKMALLYAKGKLK

SEQ ID NO: 123 - protein, 106 aa, Trypanosoma cruzi
REIMRFKKSFTCIDMHTEGEAARIVTSGLPHIPGSNMAEKKAYLQENMDYLRRGIMLEPRGHDDMFGAFLFDPIEEGADLGMVFMDTGGYLNMCGHNSIAAVTAAV

SEQ ID NO: 124 - protein, 106 aa, Trypanosoma vivax
RVVMQFTGTMTCIDMHTAGEPARIVTSGFPNIPGASLVEKRDHLQRHMDHIRRRVMLEPRGHDNMFGAFLFYPLTDGADFSVIFMDAGGYLNMCGHNSIAIATAAV

SEQ ID NO: 125 - protein, 357 aa, Trypanosoma cruzi
KREIMRFKKSFTCIDMHTEGEAARIVTSGLPHIPGSNMAEKKAYLQENMDYLRRGIMLEPRGHDDMFGAFLFDPIEEGADLGMVFMDTGGYLNMCGHNSIAAVTAAVETGIVSVPAKATNVPVVLDTPAGLVRGTAHLQSGTESEVSNASIINVPSFLYQQDVVVVLPKPYGEVRVDIAFGGNFFAIVPAEQLGIDISVQNLSRLQEAGELLRTEINRSVKVQHPQLPHINTVDCVEIYGPPTNPEANYKNVVIFGNRQADRSPCGTGTSAKMATLYAKGQLRIGETFVYESILGSLFQGRVLGEERIPGVKVPVTKDAEEGMLVVTAEITGKAFIMGFNTMLFDPTDPFKNGFTLK

SEQ ID NO: 126 - protein, 337 aa, Vibrio parahaemolyticus
KERKMRQGTFFCIDAHTCGNPVRLVAGGVPPLEGNTMSEKRQYFLEHYDWIRQALMFEPRGHSMMSGSVVLPPCSDNADASILFIETSGCLPMCGHGTIGTVTTAIENRLITPKEEGRLILDVPAGQIEVHYQTKGDKVTSVKIFNVPAYLAHQDVTVEIEGLGEITVDVAYGGNYYVIVDPQENYAGLEHYSPDEILMLSPKVRTAVSKAVECIHPNDPTVCGVSHVLWTGKPTQEGATARNAVFYGDKALDRSPCGTGTSARMAQWHAKGKLKSGEDFVHESIIGSLFNGRIEGITEVNGQTAILPSIEGWAQVYGHNTIWVDDEDPYAYGFEVK

SEQ ID NO: 127 - protein, 14 aa, Artificial Sequence
  Description of Artificial Sequence Synthetic peptide motif
DRSPCGTGTSAKMA

SEQ ID NO: 128 - protein, 5 aa, Artificial Sequence
  Description of Artificial Sequence Synthetic peptide motif
NMCGH

SEQ ID NO: 129 - protein, 6 aa, Artificial Sequence
  Description of Artificial Sequence Synthetic 6xHis tag
HHHHHH

SEQ ID NO: 130 - protein, 17 aa, Artificial Sequence
  Description of Artificial Sequence Synthetic peptide
DRSPCGXGXXAXXAXLA

SEQ ID NO: 131 - protein, 314 aa, Pseudomonas aeruginosa; identical to SEQ ID NO: 43

SEQ ID NO: 132 - DNA, 945 bp, Pseudomonas aeruginosa
atgcaacgcatccgcatcatcgattcgcataccggcggcgagcccacccgcctggtcatcggcggttttcccgatctcggccagggcgacatggctgagcgccggcgcctgctcggcgagcgccacgacgcctggcgcgccgcctgcatcctcgagccgcgcggcagcgacgtactggtcggcgccctgctctgcgcaccggtcgatcccgaagcctgtgccggagtgatcttcttcaacaacagtggctacctcggcatgtgcggccacggcaccatcggcctggtcgcctcgctggcacacctagggcggatcggtcccggcgtgcaccgcatcgagacaccggtcggcgaggtcgaggcgaccctccacgaggacggctcggtcagcgtgcgcaacgtgccggcctaccgctaccgtcgccaggtcagcgtcgaagtgcccggcatcggccgggtcagcggcgacatcgcctggggcggcaactggttcttcctggtcgccggacacggccagcgcctcgccggcgacaacctcgacgccctcaccgcctacaccgtcgccgtccagcaggcgctggacgaccaggacatccgcggcgaggacggcggcgccatcgatcacatcgaactgttcgccgacgacccccacgccgacagccgcaacttcgtgctctgccccggcaaggcctacgaccgctcgccctgcggcaccggcaccagcgccaagctggcctgcctggccgccgacggcaagctgctgcccggccagccctggcgccaggcgagcgtgatcggcagccagttcgaggggcgttacgaatggctcgacggccagcccggcgggcccatcgtgccgaccatccgcggccgcgcccacgtcagcgcggaagccaccctgctgctggcggacgacgatccgttcgcctggggcatccgccggtga

SEQ ID NO: 133 - protein, 333 aa, Brucella melitensis; identical to SEQ ID NO: 38

SEQ ID NO: 134 - DNA, 1002 bp, Brucella melitensis
atggcaagacattccttcttctgcgtcgatgggcatacatgcggcaaccctgtgcggctggtggctggcggcgggccgaacctcaatggatcgaccatgatggaaaagtgcgcgcatttcctggcggaatatgactggatccgcaccggcctcatgttcgagccgcgcggccacgacatgatgtccggttccattctctatccgccgacaaggccggattgcgacgtggcggtgctgttcatcgaaacctcgggctgcctgccgatgtgcggccatggcaccatcggcacggtgacgatggcaatcgagcagggactggtgacgcccaagacgccgggcaagctcaatctcgacacgcccgccggcctggtggcgatcgaatatgagcaggacggtcagtatgtggagcgtgtgcgcctcaccaatgtcccagctttcctttatgccgaaggcctggaggtcgaatgccccgatcttggcccgatcaaggtcgatgttgcttatggtggtaatttctacgccattgtcgaaccgcaggaaaactacacggatatggacgattactccgcgcttcagcttattgcctggagcccggtcctgcgccagcggctgaatgaaaaatacaagttccagcacccggaactgcctgatatcaaccgtctcagccatattctctggaccggaaagccgaagcacccgcaagcccatgcgcgcaatgccgtgttttatggcgacaaggctatcgaccgttcgccctgcggcaccggcacctcggcgcgcatggcccagcttgccgccaagggcaagctgaaaccgggcgatgaatttattcatgaatcgatcatcggctcgctattccatggtcgtgtggagcgcgccgccgaggttgccgggcgccccgcaattgtgccttccattgcaggctgggcgcggatgacgggctataatacgatttttatcgacgatcgcgatccgtttgctcatgggttcagcgcggcataa

SEQ ID NO: 135 - protein, 333 aa, Brucella suis
MARHSFFCVDGHTCGNPVRLVAGGGPNLNGSTMMEKRAHFLAEYDWIRTGLMFEPRGHDMMSGSILYPPTRPDCDVAVLFIETSGCLPMCGHGTIGTVTMAIEQGLVTPKTPGKLNLDTPAGLVAIEYEQDGQYVERVRLTNVPAFLYAEGLEVECPDLGPIKVDVAYGGNFYAIVEPQENYTDMDDYSALQLIAWSPVLRQRLNEKYKFQHPELPDINRLSHILWTGKPKHPQAHARNAVFYGDKAIDRSPCGTGTSARMAQLAAKGKLKPGDEFIHESIIGSLFHGRVERAAEVAGRPAIVPSIAGWARMTGYNTIFIDDRDPFAHGFSVA

SEQ ID NO: 136 - DNA, 1002 bp, Brucella suis
atggcaagacattccttcttctgcgtcgatgggcatacatgcggcaaccctgtgcggctggtggctggcggcgggccgaacctcaatggatcgaccatgatggaaaagcgcgcgcatttcctggcggaatatgactggatccgcaccggcctcatgttcgagccgcgcggccacgacatgatgtccggttccattctctatccgccgacaaggccggattgcgacgtggcggtgctgttcatcgaaacctcgggctgcctgccgatgtgcggccatggcaccatcggcacggtgacgatggcaatcgagcagggactggtgacgcccaagacgccgggcaagctcaatctcgacacgcccgccggcctggtggcgatcgaatatgagcaggacggtcagtatgtggagcgtgtgcgcctcaccaatgtcccagctttcctttatgccgaaggcctggaggtcgaatgccccgatcttggcccgatcaaggtcgatgttgcttatggtggtaatttctacgccattgtcgaaccgcaggaaaactacacggatatggacgattactccgcgcttcagcttattgcctggagcccggtcctgcgccagcggctgaatgaaaaatacaagttccagcacccggaactgcctgatatcaaccgtctcagccatattctctggaccggaaagccgaagcacccgcaagcccatgcgcgcaatgccgtgttttatggcgacaaggctatcgaccgttcgccctgcggcaccggcacgtcggcgcgcatggcccagcttgccgccaagggcaagctgaaaccgggcgatgaatttattcatgaatcgatcatcggctcgctattccatggtcgtgtggagcgcgccgccgaggttgccgggcgccccgcaattgtgccttccattgcaggctgggcgcggatgacgggctataatacgatttttatcgacgatcgcgatccgtttgctcatgggttcagcgtggcataa

SEQ ID NO: 137 - protein, 333 aa, Brucella abortus
MARHSFFCVDGHTCGNPVRLVAGGGPNLNGSTMMEKRAHFLAEYDWIRTGLMFEPRGHDMMSGSILYPPTRPDCDVAVLFIETSGCLPMCGHGTIGTVTMAIEQGLVTPKTPGKLNLDTPAGLVAIEYEQDGQYVERVRLTNVPAFLYAEGLEVECPDLGPIKVDVAYGGNFYAIVEPQENYTDMDDYSALQLIAWSPVLRQRLNEKYKFQHPELPDINRLSHILWTGKPKHPQAHARNAVFYGDKAIDRSPCGTGTSARMAQLAAKGKLKPCDEFIHESIIGSLFHGRVERAAEVAGRPAIVPSIAGWARMTGYNTIFIDDRDPFAHGFSMA

SEQ ID NO: 138 - DNA, 1002 bp, Brucella abortus
atggcaagacattccttcttctgcgtcgatgggcatacatgcggcaaccctgtgcggctggtggctggcggcgggccgaacctcaatggatcgaccatgatggaaaagcgcgcgcatttcctggcggaatatgactggatccgcaccggcctcatgttcgagccgcgcggccacgacatgatgtccggttccattctctatccgccgacaaggccggattgcgacgtggcggtgctgttcatcgaaacctcgggctgcctgccgatgtgcggccatggcaccatcggcacggtgacgatggcaatcgagcagggactggtgacgcccaagacgccgggcaagctcaatctcgacacgcccgccggcctggtggcgatcgaatatgagcaggacggtcagtatgtggagcgtgtgcgcctcaccaatgtcccagctttcctttatgccgaaggcctggaggtcgaatgccccgatcttggcccgatcaaggtcgatgttgcttatggtggtaatttctacgccattgtcgaaccgcaggaaaactacacggatatggacgattactccgcgcttcagcttattgcctggagcccggtcctgcgccagcggctgaatgaaaaatacaagttccagcacccggaactgcctgatatcaaccgtctcagccatattctctggaccggaaagccgaagcacccgcaagcccatgcgcgcaatgccgtgttttatggcgacaaggctatcgaccgttcgccctgcggcaccggcacctcggcgcgcatggcccagcttgccgccaagggcaagctgaaaccgtgcgatgaatttattcatgaatcgatcatcggctcgctattccatggtcgtgtggagcgcgccgccgaggttgccgggcgccccgcaattgtgccttccattgcaggctgggcgcggatgacgggctataatacgatttttatcgacgatcgcgatccgtttgctcatgggttcagcatggcataa

SEQ ID NO: 139 - protein, 310 aa, Burkholderia pseudomallei
MKHIHIIDSHTGGEPTRVVVSGFPALGGGTMAERLAVLAREHDRYRAACILEPRGSDVLVGALLCEPVSAGAAAGVIFFNNAGYLGMCGHGTIGLVRTLHHMGRIGPGVHRIETPVGDVEATLHDDLSVSVRNVLAYRHAKDVVVDVPGHGAVTGDVAWGGNWFFLVSDHGQRVAGENVAALAAYASAVRAALERAGVTGRDGAPIDHIELFADDPEYDSRSFVLCPGHAYDRSPCGTGTSAKLACLAADGKLAAGVTWRQASVIGSVFSASYAAAEGGVVPTIRGSAHLSAEATLVIEDDDPFGWGIAS

SEQ ID NO: 140 - DNA, 933 bp, Burkholderia pseudomallei
atgaagcacatccacatcatcgattcgcacacgggcggcgaacccacgcgcgtcgtcgtgtccggctttccggcgctcggcggcggcacgatggccgagcgtctggccgtgctcgcgcgcgagcacgaccgctaccgcgccgcctgcatcctcgagccgcgcggcagcgacgtgctcgtcggcgcgctgctgtgcgagcccgtctcggccggcgcggcggcgggcgtgatcttcttcaacaacgcgggctatctcggcatgtgcgggcacggcacgatcggcctcgtgcgcacgctgcaccacatggggcgcatcgggccgggcgtgcatcggatcgagacgccggtgggcgacgtcgaggcgacgctgcacgacgatctgtccgtgagcgtgcgcaacgtgctcgcgtaccggcacgcgaaggacgtcgtcgtcgacgtgccggggcacggcgcggtgacgggcgacgtcgcgtggggcggcaactggttcttcctcgtgagcgatcacggccagcgcgtcgcgggcgagaacgtcgcggcgctcgccgcgtacgcgtcggcggtgcgcgcggcgctcgagcgcgcgggcgtgacgggccgcgacggcgcgccgatcgatcacatcgagctgttcgccgacgatcccgagtacgacagccgcagcttcgtgctctgcccgggccacgcgtacgaccgctcgccgtgcggcaccggcacgagcgcgaagctcgcgtgcctcgccgccgacggcaagctcgcggcgggcgtgacgtggcggcaggcgagcgtgatcggcagcgtgttctcggcgagctacgcggcggccgaaggcggcgtcgtgccgacgatccgcggcagcgcgcacctgagcgcggaggcgacgctcgtgatcgaggacgacgatccgttcggctggggcatcgcatcgtga

SEQ ID NO: 141 - protein, 335 aa, Clostridium difficile [sequence omitted]

SEQ ID NO: 142 - DNA, 1008 bp, Clostridium difficile
atgaaatttagcagaagtatacaagctatagactctcatacagcaggtgaagcaactagaatagttgtaggtggaatacctaacataaaaggaaattctatgcctgagaaaaaagaatatttagaggaaaatctagattatttaagaactgctataatgttagagccaagaggacataatgatatgtttggttctgtaatgactcaaccttgttgcccagatgctgactttggaataatcttcatggatggtggtggataccttaacatgtgtggccatggtacaataggagctatgacagcagctatagaaacaggtgtagttccagcagtagagcctgtaactcatgtagttatggaagctccagcaggaataataagaggagacgttacagttgtagatggaaaagctaaagaagtatcattcttaaacgtaccagctttcttatataaagaaggtgttgaagttgacttaccaggtgttgggactgttaaatttgacatatcatttggtggaagcttctttgctataatacatgcaagtcaattaggtttaaaaatcgaacctcaaaatgctggtaaattaactgaactagctatgaaacttagagatataataaatgagaaaatagaaatacaacatccaactttagctcatataaaaactgtagacttagttgaaatatatgatgagccaactcatccagaagctacttacaaaaatgtagttatatttggtcaaggtcaagttgacagatctccatgtggaactggaacaagtgctaaattagctactttacatgctaaaggtgaattaaaagtaggagaaaaattcgtatatgaaagtatattaggaactttattcaaaggtgaaatagttgaagaaactaaagttgcagatttcaatgctgtagtacctaaaataactggttctgcttatataactggatttaatcattttgtaatagatgaagaagacccacttaaacatggatttattcttaaataa

SEQ ID NO: 143 - protein, 345 aa, Trypanosoma vivax [sequence omitted]

SEQ ID NO: 144 - DNA, 1038 bp, Trypanosoma vivax
atgcagttcaccggaacaatgacatgtattgatatgcatactgcaggtgagccagcaaggattgtcacgagtggttttcccaacatacctggtgcttcattagttgaaaaaagagaccacctccagcgacacatggaccacatacgacggagggttatgctcgagccacgtggtcatgacaacatgttcggcgcctttcttttctatccactgactgacggtgccgacttcagtgtgatattcatggacgcaggtggatatctcaatatgtgtgggcacaactctatcgccatagctacagctgctgttgagatgggaatggtgcaccccacaccagatgcgtcacagatgcctcttgttttggatactccagcaggacgtgtcctaactgatgtacatctagaatggaacaaaggggtttgcgaggtgcaccatgtggctgtgaagaatgtgccttccttcctctacatgcaagacatcgtcgttgaactcccacatccgtacggcaaagtaacggtggacatttccttcggtggtagtttctttgctcttattgacgcagcacaattacaacttacagtggacaaagggcacctgagcacacttcagcacgtgggaggtttgctgcgtgacactctgaaccgcaatgtttctgtccaacatcctcaactgccccacattaaccgtattgactgtgtggagatttacgaccctccaacaaacccagctgcgagctgtaaaaacgtcgttatatttggcaactcgcaagtggaccgttcgccttgtggaactggcacatgcgccaaaatggcgttgctgtatgcgaaggggaagttgaaggttggggatacttttgtgcacgaaagcatctcaggaaccctatttcacggtaagttgctgaatcaggtgagtatgcccggggtgaagtatccggccgttatttcagagataagtgggtcggcttacattacaggattcaacacacttctgttcgccccgcgtgatccatttcgtgacggctttacgctgagcgtgtga